(19) United States
(12) Patent Application Publication
(10) Pub. No.: US 2007/0142498 A1
(43) Pub. Date: Jun. 21, 2007

(54) DENTAL COMPOSITIONS INCLUDING THERMALLY RESPONSIVE ADDITIVES, AND THE USE THEREOF

(76) Inventors: Joan V. Brennan, Sierra Madre, CA (US); Rajdeep S. Kalgutkar, Woodbury, MN (US); Joel D. Oxman, Minneapolis, MN (US); Ajay Myer, Irvine, CA (US); Darrell S. James, Covina, CA (US); Albert I. Everaerts, Oakdale, MN (US); Lang N. Nguyen, St. Paul, MN (US); Wayne S. Mahoney, St. Paul, MN (US)

Correspondence Address: 3M INNOVATIVE PROPERTIES COMPANY, PO BOX 33427, ST. PAUL, MN, 55133-3427, US

(21) Appl. No.: 11/275,240

(22) Filed: Dec. 20, 2005

(51) Int. Cl. A61K 6/00; C09K 3/00

(52) U.S. Cl. 523/118; 106/35

(57) ABSTRACT

Hardenable and hardened dental compositions that include thermally responsive additives, and articles including such hardenable and hardened compositions, are provided. Upon heating, the hardened compositions are useful for reducing the bond strengths of orthodontic appliances adhered to tooth structures with the hardened compositions.

### BACKGROUND

Orthodontic treatment involves movement of malpositioned teeth to orthodontically correct positions. Tiny orthodontic appliances known as brackets are connected to exterior surfaces of the patient's teeth, and an archwire is placed in a slot of each bracket. The archwire forms a track to guide movement of the teeth to desired positions for correct occlusion. End sections of the archwire are often received in appliances known as buccal tubes that are fixed to the patient's molar teeth. In recent years it has become common practice to use adhesives to bond orthodontic appliances to the surface of the tooth, using either direct or indirect methods. A variety of adhesives are available to the practitioner for bonding brackets to tooth surfaces, and many offer excellent bond strengths. High bond strengths are desirable for maintaining adhesion of the bracket to the tooth surface over the duration of the treatment process, which can typically be two years or more.

However, orthodontic adhesives with high bond strengths can lead to other difficulties. For example, one of the most difficult aspects of the orthodontic treatment process can be the removal of the bracket after completion of treatment. It is well known in the industry that certain adhesives, used in combination with certain rigid brackets, are capable of causing enamel fracture under some debonding conditions. As a result, many commercially available ceramic brackets have been designed for the bond to fail at the interface between the bracket and the adhesive to prevent damage to the tooth surface during the debonding process. However, this approach results in most of the cured adhesive pad being left behind on the tooth surface after the bracket has been removed. Removal of the adhesive pad, which is typically hard and heavily crosslinked, can be time consuming for the clinician and uncomfortable for the patient.

New adhesives and methods are needed that offer satisfactory adhesion of the bracket to the tooth surface throughout the treatment process, and also allow for more convenient removal upon completion of the treatment.

### SUMMARY OF THE INVENTION

In one aspect, the present invention provides a method for reducing the bond strength of an orthodontic appliance adhered to a tooth structure with a hardened dental composition (e.g., a hardened orthodontic adhesive, a hardened orthodontic cement, and/or a hardened orthodontic primer) that includes a thermally responsive additive (e.g., a semicrystalline polymer, an amorphous polymer, a liquid crystal, and/or a wax). In one embodiment, the method includes heating the hardened dental composition (e.g., to at least 42° C.) to reduce the bond strength. Preferably, the hardened dental composition maintains sufficient bond strength prior to heating (e.g., throughout the duration of the treatment), but provides reduced bond strength upon heating, allowing for convenient removal of the orthodontic appliance from the tooth structure (e.g., less force required to debond the appliance). In some embodiments, the thermally responsive additive and/or dental composition including the same, can be placed so as to result in fracture (e.g., adhesive failure) upon debonding at an interface (e.g., an adhesive-tooth interface or an appliance-adhesive interface), or cohesive failure within the hardened dental composition upon debonding. For example, fracture at an adhesive-tooth interface can result in the hardened adhesive being substantially retained on the removed orthodontic appliance, providing for convenient clean-up of the tooth structure.

In another aspect, the present invention provides a hardenable dental composition (e.g., an orthodontic primer, an orthodontic adhesive, an orthodontic sealant, and/or an orthodontic band cement) that includes a thermally responsive additive (e.g., a semicrystalline polymer and/or an amorphous polymer). Articles having such hardenable and/or hardened dental compositions thereon are also provided, optionally as precoated articles, and optionally including one or more layers of different hardenable and/or hardened dental compositions. In one embodiment, the hardenable dental composition includes an ethylenically unsaturated compound, an initiator, a thermally responsive additive, and optionally, a filler. Optionally, the dental composition further includes a radiation-to-heat converter. In some embodiments, the hardenable dental composition can be hardened to bond an orthodontic appliance to a tooth structure with a bond strength of at least 7 MPa at room temperature measured using a shear peel test. In some embodiments, the hardenable dental composition is a self-etching orthodontic primer or a self-etching orthodontic adhesive that includes an ethylenically unsaturated compound with acid functionality. Methods for making and using such hardenable and/or hardened dental compositions, and articles having such hardenable and/or hardened dental compositions thereon, are also provided.

In another aspect, the present invention provides a packaged article including an orthodontic appliance (e.g., a precoated orthodontic appliance) at least partially surrounded by a container. The orthodontic appliance has, on the base thereof, a hardenable dental composition including an ethylenically unsaturated compound, an initiator, a filler, and a thermally responsive additive (e.g., a semicrystalline polymer, an amorphous polymer, a liquid crystal, and/or a wax). Optionally, the hardenable dental composition further includes a radiation-to-heat converter. Optionally, articles having the hardenable dental composition thereon can additionally include one or more layers of different hardenable and/or hardened dental compositions.

Definitions

As used herein, “dental composition” refers to a material (e.g., a dental or orthodontic material) capable of adhering (e.g., bonding) to a tooth structure. Dental compositions include, for example, adhesives (e.g., dental and/or orthodontic adhesives), cements (e.g., glass ionomer cements, resin-modified glass ionomer cements, and/or orthodontic cements), primers (e.g., orthodontic primers), restoratives, liners, sealants (e.g., orthodontic sealants), and coatings. Oftentimes a dental composition can be used to bond a dental article to a tooth structure.

As used herein, “dental article” refers to an article that can be adhered (e.g., bonded) to a tooth structure. Dental articles include, for example, crowns, bridges. veneers, inlays, onlays, fillings, orthodontic appliances and devices, and prostheses (e.g., partial or full dentures).

As used herein, “orthodontic appliance” refers to any device intended to be bonded to a tooth structure, including, but not limited to, orthodontic brackets, buccal tubes, lingual retainers, orthodontic bands, bite openers, buttons, and cleats. The appliance has a base for receiving adhesive and it can be a flange made of metal, plastic, ceramic, or combinations thereof. Alternatively, the base can be a custom base formed from cured adhesive layer(s) (i.e., single or multi-layer adhesives).

As used herein, a “packaged” article refers to an orthodontic appliance or card that is received in a container. Preferably, the container provides protection from environmental conditions including, for example, moisture and light.

As used herein, a “release” substrate refers to a substrate in contact with an article that is removed from the article before or during use of the article.

As used herein, a “radiation-to-heat converter” refers to a material or composition that absorbs incident radiation (e.g., visible light, ultraviolet (UV) radiation, infrared (IR) radiation, near infrared (NIR) radiation, and/or radio frequency (RF) radiation) and converts a substantial portion (e.g., at least 50%) of the incident radiation into heat, which can be useful for softening the thermally responsive additive.

As used herein, “softening” refers to loss of modulus of a material that can occur as a result of physical and/or chemical changes in the material. The degree of softness or deformability of a material is sometimes referred to as “compliance” of the material, wherein compliance is defined as the inverse of the Young's modulus of the material.

As used herein, “tooth structure” refers to surfaces including, for example, natural and artificial tooth surfaces, bone, tooth models, and the like.

As used herein, a “multi-layer” adhesive refers to an adhesive having two or more distinctly different layers (i.e., layers differing in composition, and preferably having different chemical and/or physical properties).

As used herein, a “layer” refers to a discontinuous (e.g., a patterned layer) or continuous (e.g., non-patterned) material extending across all or a portion of a material different than the layer. The layer may be of uniform or varying thickness.

As used herein, a “patterned layer” refers to a discontinuous material extending across (and optionally attached to) only selected portions of a material different than the patterned layer.

As used herein, a “non-patterned layer” refers to a continuous material extending across (and optionally attached to) an entire portion of a material different than the non-patterned layer.

In general, a layer “on,” “extending across,” or “attached to” another material different than the layer is intended to be broadly interpreted to optionally include one or more additional layers between the layer and the material different than the layer.

As used herein, “hardenable” is descriptive of a material or composition that can be cured (e.g., polymerized or crosslinked) or solidified, for example, by removing solvent (e.g., by evaporation and/or heating); heating to induce polymerization and/or crosslinking; irradiating to induce polymerization and/or crosslinking; and/or by mixing one or more components to induce polymerization and/or crosslinking. “Mixing” can be performed, for example, by combining two or more parts and mixing to form a homogeneous composition. Alternatively, two or more parts can be provided as separate layers that intermix (e.g., spontaneously or upon application of shear stress) at the interface to initiate polymerization.

As used herein, “hardened” refers to a material or composition that has been cured (e.g., polymerized or crosslinked) or solidified.

As used herein, “hardener” refers to something that initiates hardening of a resin. A hardener may include, for example, a polymerization initiator system, a photoinitiator system, and/or a redox initiator system.

As used herein, “photobleachable” refers to loss of color upon exposure to actinic radiation.

As used herein, the term “(meth)acrylate” is a shorthand reference to acrylate, methacrylate, or combinations thereof, and “(meth)acrylic” is a shorthand reference to acrylic, methacrylic, or combinations thereof.

As used herein, the chemical term “group” allows for substitution. As used herein, “a” or “an” means one or more.

### BRIEF DESCRIPTION OF THE DRAWINGS

FIG. 1 is perspective view of an orthodontic appliance having a hardenable or hardened dental composition of the present invention on the base thereof.

FIG. 2 is a side view of the orthodontic appliance of FIG. 1.

FIG. 3 is a perspective view of a packaged article illustrating an orthodontic appliance having a hardenable or hardened dental composition of the present invention on the base thereof in a container in which the cover has been partially opened.

FIGS. 4-6 are side views of orthodontic appliances having a plurality of layers on the bases thereof, in which at least one layer of the plurality of layers is a hardenable or hardened dental composition of the present invention.

FIG. 7a illustrates scanning electron micrographs of poly(caprolactone) (PCL) nanofibers. FIG. 7b is a higher magnification view of the nanofibers illustrated in FIG. 7a.

FIG. 8a illustrates scanning electron micrographs of poly(caprolactone) (PCL) nanofibers including indium-tin oxide (ITO) particles. FIG. 8b is a higher magnification view of the nanofibers illustrated in FIG. 8a.

FIG. 9a illustrates scanning electron micrographs of poly(propylene carbonate), (PPC) nanofibers. FIG. 9b is a higher magnification view of the nanofibers illustrated in FIG. 9a.

FIG. 10a illustrates scanning electron micrographs of poly(caprolactone) (PCL) nanofibers including a near infrared (NIR) absorber dye. FIG. 10b is a higher magnification view of the nanofibers illustrated in FIG. 10a.

### DETAILED DESCRIPTION OF CERTAIN EMBODIMENTS

The present invention provides hardenable dental compositions, and articles including such compositions, that are capable of adhering to a tooth structure upon hardening. Further, the adherence (e.g., bond strength) to the tooth structure of such hardened compositions can be reduced upon heating, typically under convenient conditions. The reduced adherence can be useful if and when it is desired to remove the hardened composition from the tooth structure. Such hardenable dental compositions encompass materials (e.g., dental and/or orthodontic materials) capable of adhering (e.g., bonding) to a tooth structure, such as adhesives (e.g., dental and/or orthodontic adhesives), cements (e.g., glass ionomer cements, resin-modified glass ionomer cements), primers, restoratives, liners, sealants, and coatings. Oftentimes a dental composition can be used to bond a dental article (e.g., an orthodontic appliance) to a tooth structure.

In some embodiments, such hardenable dental compositions can, upon hardening, provide sufficient bond strength to adhere an orthodontic appliance to a tooth structure during orthodontic treatment, and are further useful for reducing the bond strength, for example, at the end of the treatment process when it is necessary for the practitioner to remove the appliance from the tooth structure. The compositions, articles, and methods are designed to reduce the bond strength upon heating of the hardened dental composition under convenient conditions. The resulting reduced bond strength can allow for convenient removal of not only the orthodontic appliance, but also for any hardened dental composition remaining on the tooth structure after removal of the appliance.

Hardenable and hardened dental compositions of the present invention include thermally responsive additives, which are described in detail herein. As used herein, a “thermally responsive additive” refers to an additive that softens upon heating. As used herein, an “additive” to a dental composition refers to a portion of the dental composition that when combined with another material provides the dental composition. Thus, an additive itself cannot be the dental composition.

A thermally responsive additive can be incorporated into a wide variety of dental compositions (e.g., dental and orthodontic materials) including, for example, adhesives, cements (e.g., glass ionomer cements, resin-modified glass ionomer cements), primers, restoratives, liners, sealants, and coatings at levels effective to decrease bond strength of the hardened composition upon heating, while maintaining sufficient adhesion (e.g., of an orthodontic appliance) to the tooth structure during treatment. Treatment can include dental and/or orthodontic treatment processes that last a month, a year, two years, or even longer.

For certain embodiments, such dental compositions can be conveniently applied to the base of an orthodontic appliance by a practitioner. Alternatively, orthodontic appliances can be provided having such dental compositions precoated on the base of the appliance. Typically such precoated appliances are provided as packaged articles with or without a release liner or foam pad liner such as those described, for example, in U.S. Pat. No. 6,183,249 (Brennan et al.). Exemplary containers are well known in the art and are disclosed, for example, in U.S. Pat. No. 5,172,809 (Jacobs et al.) and U.S. Pat. No. 6,089,861 (Kelly et al.).

Hardenable dental compositions of the present invention typically include an ethylenically unsaturated compound, an initiator, and a thermally responsive additive. In some embodiments, the hardenable dental composition also includes a filler. In some embodiments, the hardenable dental composition further includes an ethylenically unsaturated compound with acid functionality, wherein the hardenable dental composition can be, for example, a self-etching orthodontic primer or a self-etching orthodontic adhesive. In some embodiments, the hardenable dental composition further includes a radiation-to-heat converter as described hereinafter. Optionally, the hardenable dental composition of the present invention can include a thermally labile component; and/or an acid-generating component and an acid-reactive component as described hereinafter. Preferably, such compositions, upon hardening, can bond an orthodontic appliance to a tooth structure with a bond strength (using the shear peel test method described herein) of at least 7 MPa at room temperature.

Thermally Responsive Additives

Hardenable and hardened dental compositions of the present invention include a thermally responsive additive. As used herein, a “thermally responsive additive” is meant to include an additive that softens upon heating to a temperature (e.g., no greater than 200° C., preferably no greater than 150° C., more preferably no greater than 100° C., and most preferably no greater than 80° C.) that is below the decomposition temperature of the additive. Specifically, upon heating to an elevated temperature (i.e., at least 42° C.), the storage modulus of the additive at the elevated temperature decreases compared to the storage modulus of the additive at room temperature (e.g., 25° C.). Preferably, the storage modulus of the additive at the elevated temperature is at most 80%, more preferably at most 60%, 40%, 20%, 10%, 5%, 2%, 1%, 0.1%, or even 0.01% of the storage modulus of the additive at room temperature. Methods of measuring storage modulus of materials at specified temperatures are well known in the art and include those described, for example, in Rudin, “The Elements of Polymer Science and Engineering,” 2^{nd} Ed, Chapter 11, pp. (1999). Such methods include, for example, dynamic mechanical measurements by techniques such as dynamic mechanical analysis (DMA).

Such thermally responsive additives typically have a maximum in the rate of storage modulus decrease occurring typically within the range of 42° C. to 200° C. Such a maximum in the rate of storage modulus decrease can correspond to transitions including, for example, melt transitions (Tₘ), glass transitions (T_{g}), solid to smectic or nematic phase transitions in liquid crystals, isotropic melt transitions in liquid crystals, and the like.

In certain embodiments, softening of a hardened dental composition including a thermally responsive additive, upon heating to a temperature (e.g., no greater than 200° C., preferably no greater than 150° C., more preferably no greater than 100° C., and most preferably no greater than 80° C.) that is below the decomposition temperature of the additive, may optionally, but not necessarily, be observed to a greater extent than for the hardened dental composition not including a thermally responsive additive under similar conditions.

Typically and preferably, a hardened dental composition including a thermally responsive additive shows lower bond strength at an elevated temperature (e.g., no greater than 200° C., preferably no greater than 150° C., more preferably no greater than 100° C., and most preferably no greater than 80° C.) that is below the decomposition temperature of the additive. Specifically, upon heating to an elevated temperature (i.e., at least 42° C.), the bond strength of the hardened dental composition at the elevated temperature decreases compared to the bond strength of the hardened dental composition not including the thermally responsive additive at the same elevated temperature. Preferably, the bond strength of the dental composition at the elevated temperature (e.g., 70° C.) is at most 90%, more preferably at most 80%, 50%, 30%, 20%, or even 10% of the bond strength of the hardened dental composition not including the thermally responsive additive at the same elevated temperature (e.g., 70° C.). Further, in certain embodiments it is preferred that bond strengths at the elevated temperature be maintained at a sufficient level (e.g., to avoid having brackets falling off into the patient's mouth before pressure is applied by the practitioner). In such embodiments, it is preferred that the bond strength of the dental composition at the elevated temperature (e.g., upon exposure to hot foods) is at least 5 MPa at the elevated temperature.

Preferably, dental compositions including at most 50%, more preferably at most 30%, 30%, 10%, 5%, or even 1% by weight loading of the thermally responsive additive can exhibit such losses in storage modulus and/or bond strength at an elevated temperature. Further, at the same loadings of thermally responsive additive, preferably the bond strength of the hardened dental composition at room temperature (e.g., 25° C.) is at least 50%, more preferably at least 70%, 90%, 100%, or even greater than 100% of the bond strength of the hardened dental composition not including the thermally responsive additive at the same temperature.

In some embodiments, thermally responsive additives can be polymers. Polymers having a wide variety of morphologies can be used. For example, a thermally responsive additive can be a semicrystalline polymer, an amorphous polymer, or a combination thereof. In some embodiments, thermally responsive additives can be liquid crystals (e.g., non-polymeric liquid crystals or polymeric liquid crystals). In some embodiments, thermally responsive additives can be waxes.

Useful semicrystalline polymers typically have a melt transition temperature (Tₘ) of at least 42° C. Useful semicrystalline polymers typically have a melt transition temperature (Tₘ) of at most 200° C., preferably at most 150° C., more preferably at most 100° C., and most preferably no greater than 80° C.

Useful amorphous polymers typically have a glass transition temperature (T_{g}) of at least 42° C. Useful amorphous polymers typically have a glass transition temperature (T_{g}) of at most 200° C., preferably at most 150° C., more preferably at most 100° C., and most preferably no greater than 80° C.

Examples of polymer classes that can be used for thermally responsive additives include poly((meth)acrylics), poly((meth)acrylamides), poly(alkenes), poly(dienes), poly(styrenes), poly(vinyl alcohol), poly(vinyl ketones), poly(vinyl esters), poly(vinyl ethers), poly(vinyl thioethers), poly(vinyl halides), poly(vinyl nitriles), poly(phenylenes), poly(anhydrides), poly(carbonates), poly(esters), poly(lactones), poly(ether ketones), poly(alkylene oxides), poly(urethanes), poly(siloxanes), poly(sulfides), poly(sulfones), poly(sulfonamides), poly(thioesters), poly(amides), poly(anilines), poly(imides), poly(imines), poly(ureas), poly(phosphazenes), poly(silanes), poly(silazanes), carbohydrates, gelatins, poly(acetals), poly(benzoxazoles), poly(carboranes), poly(oxadiazoles), poly(piperazines), poly(piperidines), poly(pyrazoles), poly(pyridines), poly(pyrrolidines), poly(triazines), and combinations thereof. One of skill in the art could select, without undue experimentation, polymers from the above-recited classes that have desired transition temperatures. See, for example, “Polymer Handbook,” 4^{th} Edition edited by J. Brandrup et al. (1999) for a list of melt transition temperatures and glass transition temperatures of selected polymers.

A wide variety of liquid crystals can be used for thermally responsive additives including, for example, those recited in “Liquid Crystals Handbook,” volumes 1-3, edited by Demus et al. (1998). Suitable liquid crystals typically have an isotropic transition temperature of at least 42° C. Suitable liquid crystals typically have an isotropic transition temperature of at most 200° C., preferably at most 150° C., more preferably at most 100° C., and most preferably no greater than 80° C. One of skill in the art could select, without undue experimentation, liquid crystals that have desired transition temperatures.

Useful classes of liquid crystals include, for example, biphenyls (e.g., R—Ph—Ph—CN); terphenyls (e.g., R—Ph—Ph—Ph—CN); esters (e.g., R—PhC(O)O-—Ph—OR′, R—PhC(O)O—Ph—CN, and R—PhC(O)O—Ph—Ph—CN); tolanes (e.g., R—Ph—C≡C—Ph—OR′); Schiff's bases (e.g., R—Ph—N═CH—Ph—OR′ and R—O—Ph—CH═N—Ph—CN); azo compounds (R—Ph—N═N—Ph—OR′); azoxy compounds (e.g., R—Ph—N═N⁺(O⁻)—Ph—OR′); and stilbenes (e.g., R—Ph—C(Cl)═CH—Ph—OR′), where each R and R′ independently represent an alkyl group. R is preferably a higher alkyl group, and typically at least a C7 alkyl group, and sometimes at least a C12 alkyl group. R′ is preferably a lower alkyl group, and typically a C1 or C2 alkyl group.

Examples of waxes that can be used for thermally responsive additives include dental waxes such as pattern wax, base-plate wax, sheet wax, impression wax, study wax, polycaprolactone, polyvinylacetate, ethylene-vinyl acetate copolymer, polyethylene glycol, esters of carboxylic acids with long chain alcohols (e.g., behenyl acrylate), esters of long chain carboxylic acids with long chain alcohols (e.g., beeswax, a non-polymeric wax), petroleum waxes, oxidized polyethylene wax (e.g., a wax available under the trade designation CERIDUST 3719 from Clariant Corp., Charlotte, N.C.), micronized, polar, high density polyethylene wax (e.g., a wax available under the trade designation CERIDUST 3731 from Clariant Corp., Charlotte, N.C.), camauba wax (e.g., a wax available under the trade designation MIWAX from Michelman Incorporated, Cincinnati, Ohio), and combinations thereof (e.g., blends including two or more of microcystalline waxes, carnauba wax, ceresin, and beeswax). Useful waxes can also be oligomeric or polymeric. Useful waxes can be macrocrystalline or microcrystalline, natural or synthetic, and they may contain functional groups (e.g., carboxyl, alcohol, ester, ketone, and/or amide groups). Suitable waxes melt at or above room temperature (e.g., 25° C.), and typically at or above 40° C., and sometimes at or above 50° C. Suitable waxes typically have low melt temperatures (e.g., no greater than 200° C., preferably no greater than 150° C., more preferably no greater than 100° C., even more preferably no greater than 90° C., and most preferably no greater than 80° C.). Suitable waxes can have a wide variety of physical properties. For example, at room temperature, physical properties of suitable waxes can range from kneadable to hard or brittle; coarse to crystalline; and/or transparent to opaque (with transparent being preferred).

Thermally responsive additives can preferably be incorporated into dental compositions of the present invention at levels effective to decrease the bond strength of the hardened dental composition upon heating to the desired temperature. Preferably, such levels of the additive also allow for sufficient adhesion during treatment process. Although levels of thermally responsive additive will depend on the specific dental composition being used, typically the hardenable dental composition will include at least 0.01% , preferably at least 0.1%, 1%, 5%, 10%, 20%, 30%, 40%, or even 50% by weight additive, based on the total weight of the dental composition. In some embodiments, the dental composition will include at most 85%, preferably at most 80%, 75%, 70%, or even 65% by weight additive, based on the total weight of the dental composition.

Thermally responsive additives can be in a wide variety of forms including, for example, particles, powders, fibers, disks, plates, flakes, tubes, films, or combinations thereof. Typically the additive is in the form of a powder, which preferably has an average particle size of at most 10 micrometers. As used herein for non-spherical particles, “particle size” refers to the smallest dimension of the particle.

In some embodiments, the thermally responsive additive is distributed uniformly throughout the hardenable and/or hardened dental composition. In other embodiments, especially for embodiments in which the hardenable dental composition is precoated on the base of an orthodontic appliance, the additive can be concentrated in a portion of the hardenable dental composition. For example, the additive can be concentrated near one surface (e.g., the outer surface that will contact the tooth structure) to influence the fracture to occur near the tooth structure upon debonding. Additives concentrated near one surface is meant to include additives adhered to a surface of the hardenable or hardened dental composition.

Hardenable Component

The hardenable dental compositions of the present invention typically include a hardenable (e.g., polymerizable) component, thereby forming hardenable (e.g., polymerizable) compositions. The hardenable component can include a wide variety of chemistries, such as ethylenically unsaturated compounds (with or without acid functionality), epoxy (oxirane) resins, vinyl ethers, photopolymerization systems, redox cure systems, glass ionomer cements, polyethers, polysiloxanes, and the like. In some embodiments, the compositions can be hardened (e.g., polymerized by conventional photopolymerization and/or chemical polymerization techniques) prior to applying the hardened dental composition. In other embodiments, a dental composition can be hardened (e.g., polymerized by conventional photopolymerization and/or chemical polymerization techniques) after applying the dental composition.

In certain embodiments, the compositions are photopolymerizable, i.e., the compositions contain a photoinitiator (i.e., a photoinitiator system) that upon irradiation with actinic radiation initiates the polymerization (or hardening) of the composition. Such photopolymerizable compositions can be free radically polymerizable or cationically polymerizable. In other embodiments, the compositions are chemically hardenable, i.e., the compositions contain a chemical initiator (i.e., initiator system) that can polymerize, cure, or otherwise harden the composition without dependence on irradiation with actinic radiation. Such chemically hardenable compositions are sometimes referred to as “self-cure” compositions and may include glass ionomer cements (e.g., conventional and resin-modified glass ionomer cements), redox cure systems, and combinations thereof.

Suitable photopolymerizable components that can be used in the dental compositions of the present invention include, for example, epoxy resins (which contain cationically active epoxy groups), vinyl ether resins (which contain cationically active vinyl ether groups), ethylenically unsaturated compounds (which contain free radically active unsaturated groups, e.g., acrylates and methacrylates), and combinations thereof. Also suitable are polymerizable materials that contain both a cationically active functional group and a free radically active functional group in a single compound. Examples include epoxy-functional acrylates, epoxy-functional methacrylates, and combinations thereof.

Ethylenically Unsaturated Compounds

The compositions of the present invention may include one or more hardenable components in the form of ethylenically unsaturated compounds with or without acid functionality, thereby forming hardenable compositions.

Suitable hardenable compositions may include hardenable components (e.g., photopolymerizable compounds) that include ethylenically unsaturated compounds (which contain free radically active unsaturated groups). Examples of useful ethylenically unsaturated compounds include acrylic acid esters, methacrylic acid esters, hydroxy-functional acrylic acid esters, hydroxy-functional methacrylic acid esters, and combinations thereof.

The compositions (e.g., photopolymerizable compositions) may include compounds having free radically active functional groups that may include monomers, oligomers, and polymers having one or more ethylenically unsaturated group. Suitable compounds contain at least one ethylenically unsaturated bond and are capable of undergoing addition polymerization. Such free radically polymerizable compounds include mono-, di- or poly-(meth)acrylates (i.e., acrylates and methacrylates) such as, methyl (meth)acrylate, ethyl acrylate, isopropyl methacrylate, n-hexyl acrylate, stearyl acrylate, allyl acrylate, glycerol triacrylate, ethyleneglycol diacrylate, diethyleneglycol diacrylate, triethyleneglycol dimethacrylate, 1,3-propanediol di(meth)acrylate, trimethylolpropane triacrylate, 1,2,4-butanetriol trimethacrylate, 1,4-cyclohexanediol diacrylate, pentaerythritol tetra(meth)acrylate, sorbitol hexacrylate, tetrahydrofurfuryl (meth)acrylate, bis[1-(2-acryloxy)]-p-ethoxyphenyldimethylmethane, bis[1-(3-acryloxy-2-hydroxy)]-p-propoxyphenyldimethylmethane, ethoxylated bisphenolA di(meth)acrylate, and trishydroxyethyl-isocyanurate trimethacrylate; (meth)acrylamides (i.e., acrylamides and methacrylamides) such as (meth)acrylamide, methylene bis-(meth)acrylamide, and diacetone (meth)acrylamide; urethane (meth)acrylates; the bis-(meth)acrylates of polyethylene glycols (preferably of molecular weight 200-500), copolymerizable mixtures of acrylated monomers such as those in U.S. Pat. No. 4,652,274 (Boettcher et al.), acrylated oligomers such as those of U.S. Pat. No. 4,642,126 (Zador et al.), and poly(ethylenically unsaturated) carbamoyl isocyanurates such as those disclosed in U.S. Pat. No. 4,648,843 (Mitra); and vinyl compounds such as styrene, diallyl phthalate, divinyl succinate, divinyl adipate and divinyl phthalate. Other suitable free radically polymerizable compounds include siloxane-functional (meth)acrylates as disclosed, for example, in WO-00/38619 (Guggenberger et al.), WO-01/92271 (Weinmann et al.), WO-01/07444 (Guggenberger et al.), WO-00/42092 (Guggenberger et al.) and fluoropolymer-functional (meth)acrylates as disclosed, for example, in U.S. Pat. No. 5,076,844 (Fock et al.), U.S. Pat. No. 4,356,296 (Griffith et al.), EP-0373 384 (Wagenknecht et al.), EP-0201 031 (Reiners et al.), and EP-0201 778 (Reiners et al.). Mixtures of two or more free radically polymerizable compounds can be used if desired.

The hardenable component may also contain hydroxyl groups and ethylenically unsaturated groups in a single molecule. Examples of such materials include hydroxyalkyl (meth)acrylates, such as 2-hydroxyethyl (meth)acrylate and 2-hydroxypropyl (meth)acrylate; glycerol mono- or di-(meth)acrylate; trimethylolpropane mono- or di-(meth)acrylate; pentaerythritol mono-, di-, and tri-(meth)acrylate; sorbitol mono-, di-, tri-, tetra-, or penta-(meth)acrylate; and 2,2-bis[4-(2-hydroxy-3-ethacryloxypropoxy)phenyl]propane (bisGMA). Suitable ethylenically unsaturated compounds are also available from a wide variety of commercial sources, such as Sigma-Aldrich, St. Louis. Mixtures of ethylenically unsaturated compounds can be used if desired.

In certain embodiments hardenable components include PEGDMA (polyethyleneglycol dimethacrylate having a molecular weight of approximately 400), bisGMA, UDMA (urethane dimethacrylate), GDMA (glycerol dimethacrylate), TEGDMA (triethyleneglycol dimethacrylate), bisEMA6 as described in U.S. Pat. No. 6,030,606 (Holmes), and NPGDMA (neopentylglycol dimethacrylate). Various combinations of the hardenable components can be used if desired.

Preferably, compositions of the present invention include at least 5% by weight, more preferably at least 10% by weight, and most preferably at least 15% by weight ethylenically unsaturated compounds, based on the total weight of the unfilled composition. Preferably, compositions of the present invention include at most 95% by weight, more preferably at most 90% by weight, and most preferably at most 80% by weight ethylenically unsaturated compounds, based on the total weight of the unfilled composition.

Preferably, compositions of the present invention include ethylenically unsaturated compounds without acid functionality. Preferably, compositions of the present invention include at least 5% by weight (wt-%), more preferably at least 10% by weight, and most preferably at least 15% by weight ethylenically unsaturated compounds without acid functionality, based on the total weight of the unfilled composition. Preferably, compositions of the present invention include at most 95% by weight, more preferably at most 90% by weight, and most preferably at most 80% by weight ethylenically unsaturated compounds without acid functionality, based on the total weight of the unfilled composition.

Ethylenically Unsaturated Compounds with Acid Functionality

The compositions of the present invention may include one or more hardenable components in the form of ethylenically unsaturated compounds with acid functionality, thereby forming hardenable compositions.

As used herein, ethylenically unsaturated compounds with acid functionality is meant to include monomers, oligomers, and polymers having ethylenic unsaturation and acid and/or acid-precursor functionality. Acid-precursor functionalities include, for example, anhydrides, acid halides, and pyrophosphates. The acid functionality can include carboxylic acid functionality, phosphoric acid functionality, phosphonic acid functionality, sulfonic acid functionality, or combinations thereof.

Ethylenically unsaturated compounds with acid functionality include, for example, α,β-unsaturated acidic compounds such as glycerol phosphate mono(meth)acrylates, glycerol phosphate di(meth)acrylates, hydroxyethyl (meth)acrylate (e.g., HEMA) phosphates, bis((meth)acryloxyethyl) phosphate, ((meth)acryloxypropyl) phosphate, bis((meth)acryloxypropyl) phosphate, bis((meth)acryloxy)propyloxy phosphate, (meth)acryloxyhexyl phosphate, bis((meth)acryloxyhexyl) phosphate, (meth)acryloxyoctyl phosphate, bis((meth)acryloxyoctyl) phosphate, (meth)acryloxydecyl phosphate, bis((meth)acryloxydecyl) phosphate, caprolactone methacrylate phosphate, citric acid di- or tri-methacrylates, poly(meth)acrylated oligomaleic acid, poly(meth)acrylated polymaleic acid, poly(meth)acrylated poly(meth)acrylic acid, poly(meth)acrylated polycarboxyl-polyphosphonic acid, poly(meth)acrylated polychlorophosphoric acid, poly(meth)acrylated polysulfonate, poly(meth)acrylated polyboric acid, and the like, may be used as components in the hardenable component system. Also monomers, oligomers, and polymers of unsaturated carbonic acids such as (meth)acrylic acids, aromatic (meth)acrylated acids (e.g., methacrylated trimellitic acids), and anhydrides thereof can be used. Certain preferred compositions of the present invention include an ethylenically unsaturated compound with acid functionality having at least one P—OH moiety.

Certain of these compounds are obtained, for example, as reaction products between isocyanatoalkyl (meth)acrylates and carboxylic acids. Additional compounds of this type having both acid-functional and ethylenically unsaturated components are described in U.S. Pat. No. 4,872,936 (Engelbrecht) and U.S. Pat. No. 5,130,347 (Mitra). A wide variety of such compounds containing both the ethylenically unsaturated and acid moieties can be used. Mixtures of such compounds can be used if desired.

Additional ethylenically unsaturated compounds with acid functionality include, for example, polymerizable bisphosphonic acids as disclosed for example, in U.S. Pat. Publication No. 2004/0206932 (Abuelyaman et al.); AA:ITA:IEM (copolymer of acrylic acid:itaconic acid with pendent methacrylate made by reacting AA:ITA copolymer with sufficient 2-isocyanatoethyl methacrylate to convert a portion of the acid groups of the copolymer to pendent methacrylate groups as described, for example, in Example 11 of U.S. Pat. No. 5,130,347 (Mitra)); and those recited in U.S. Pat. No. 4,259,075 (Yamauchi et al.), U.S. Pat. No. 4,499,251 (Omura et al.), U.S. Pat. No. 4,537,940 (Omura et al.), U.S. Pat. No. 4,539,382 (Omura et al.), U.S. Pat. No. 5,530,038 (Yamamoto et al.), U.S. Pat. No. 6,458,868 (Okada et al.), and European Pat. Application Publication Nos. EP 712,622 (Tokuyama Corp.) and EP 1,051,961 (Kuraray Co., Ltd.).

Compositions of the present invention can also include compositions that include combinations of ethylenically unsaturated compounds with acid functionality. Preferably the compositions are self-adhesive and are non-aqueous. For example, such compositions can include: a first compound including at least one (meth)acryloxy group and at least one —O—P(O)(OH)ₓ group, wherein x=1 or 2, and wherein the at least one —O—P(O)(OH)ₓ group and the at least one (meth)acryloxy group are linked together by a C1-C4 hydrocarbon group; a second compound including at least one (meth)acryloxy group and at least one —O—P(O)(OH)ₓ group, wherein x=1 or 2, and wherein the at least one —O—P(O)(OH)ₓ group and the at least one (meth)acryloxy group are linked together by a C5-C12 hydrocarbon group; an ethylenically unsaturated compound without acid functionality; an initiator system; and a filler. Such compositions are described, for example, in U.S. Provisional Application Ser. No. 60/600,658 (Luchterhandt et al.), filed on Aug. 11, 2004.

Preferably, the compositions of the present invention include at least 1% by weight, more preferably at least 3% by weight, and most preferably at least 5% by weight ethylenically unsaturated compounds with acid functionality, based on the total weight of the unfilled composition. Preferably, compositions of the present invention include at most 80% by weight, more preferably at most 70% by weight, and most preferably at most 60% by weight ethylenically unsaturated compounds with acid functionality, based on the total weight of the unfilled composition.

Epoxy (Oxirane) or Vinyl Ether Compounds

The hardenable compositions of the present invention may include one or more hardenable components in the form of epoxy (oxirane) compounds (which contain cationically active epoxy groups) or vinyl ether compounds (which contain cationically active vinyl ether groups), thereby forming hardenable compositions.

The epoxy or vinyl ether monomers can be used alone as the hardenable component in a dental composition or in combination with other monomer classes, e.g., ethylenically unsaturated compounds as described herein, and can include as part of their chemical structures aromatic groups, aliphatic groups, cycloaliphatic groups, and combinations thereof.

Examples of epoxy (oxirane) compounds include organic compounds having an oxirane ring that is polymerizable by ring opening. These materials include monomeric epoxy compounds and epoxides of the polymeric type and can be aliphatic, cycloaliphatic, aromatic or heterocyclic. These compounds generally have, on the average, at least 1 polymerizable epoxy group per molecule, in some embodiments at least 1.5, and in other embodiments at least 2 polymerizable epoxy groups per molecule. The polymeric epoxides include linear polymers having terminal epoxy groups (e.g., a diglycidyl ether of a polyoxyalkylene glycol), polymers having skeletal oxirane units (e.g., polybutadiene polyepoxide), and polymers having pendent epoxy groups (e.g., a glycidyl methacrylate polymer or copolymer). The epoxides may be pure compounds or may be mixtures of compounds containing one, two, or more epoxy groups per molecule. The “average” number of epoxy groups per molecule is determined by dividing the total number of epoxy groups in the epoxy-containing material by the total number of epoxy-containing molecules present.

These epoxy-containing materials may vary from low molecular weight monomeric materials to high molecular weight polymers and may vary greatly in the nature of their backbone and substituent groups. Illustrative of permissible substituent groups include halogens, ester groups, ethers, sulfonate groups, siloxane groups, carbosilane groups, nitro groups, phosphate groups, and the like. The molecular weight of the epoxy-containing materials may vary from 58 to 100,000 or more.

Suitable epoxy-containing materials useful as the resin system reactive components in the present invention are listed in U.S. Pat. No. 6,187,836 (Oxman et al.) and U.S. Pat. No. 6,084,004 (Weinmann et al.).

Other suitable epoxy resins useful as the resin system reactive components include those which contain cyclohexene oxide groups such as epoxycyclohexanecarboxylates, typified by 3,4-epoxycyclohexylmethyl-3,4-epoxycyclohexanecarboxylate, 3,4-epoxy-2-methylcyclohexylmethyl-3,4-epoxy-2-methylcyclohexane carboxylate, and bis(3,4-epoxy-6-methylcyclohexyl-methyl) adipate. For a more detailed list of useful epoxides of this nature, reference is made to U.S. Pat. No. 6,245,828 (Weinmann et al.) and U.S. Pat. No. 5,037,861 (Crivello et al.); and U.S. Pat. Publication No. 2003/035899 (Klettke et al.).

Other epoxy resins that may be useful in the compositions of this invention include glycidyl ether monomers. Examples are glycidyl ethers of polyhydric phenols obtained by reacting a polyhydric phenol with an excess of chlorohydrin such as epichlorohydrin (e.g., the diglycidyl ether of 2,2-bis-(2,3-epoxypropoxyphenol)propane). Further examples of epoxides of this type are described in U.S. Pat. No. 3,018,262 (Schroeder), and in “Handbook of Epoxy Resins” by Lee and Neville, McGraw-Hill Book Co., New York (1967).

Other suitable epoxides useful as the resin system reactive components are those that contain silicon, useful examples of which are described in International Pat. Publication No. WO 01/51540 (Klettke et al.).

Additional suitable epoxides useful as the resin system reactive components include octadecylene oxide, epichlorohydrin, styrene oxide, vinyl cyclohexene oxide, glycidol, glycidylmethacrylate, diglycidyl ether of Bisphenol A and other commercially available epoxides, as provided in U.S. Ser. No. 10/719,598 (Oxman et al.; filed Nov. 21, 2003).

Blends of various epoxy-containing materials are also contemplated. Examples of such blends include two or more weight average molecular weight distributions of epoxy-containing compounds, such as low molecular weight (below 200), intermediate molecular weight (200 to 10,000) and higher molecular weight (above 10,000). Alternatively or additionally, the epoxy resin may contain a blend of epoxy-containing materials having different chemical natures, such as aliphatic and aromatic, or functionalities, such as polar and non-polar.

Other types of useful hardenable components having cationically active functional groups include vinyl ethers, oxetanes, spiro-orthocarbonates, spiro-orthoesters, and the like.

If desired, both cationically active and free radically active functional groups may be contained in a single molecule. Such molecules may be obtained, for example, by reacting a di- or poly-epoxide with one or more equivalents of an ethylenically unsaturated carboxylic acid. An example of such a material is the reaction product of UVR-6105 (available from Union Carbide) with one equivalent of methacrylic acid. Commercially available materials having epoxy and free-radically active functionalities include the CYCLOMER series, such as CYCLOMER M-100, M-101, or A-200 available from Daicel Chemical, Japan, and EBECRYL-3605 available from Radcure Specialties, UCB Chemicals, Atlanta, Ga.

The cationically curable components may further include a hydroxyl-containing organic material. Suitable hydroxyl-containing materials may be any organic material having hydroxyl functionality of at least 1, and preferably at least 2. Preferably, the hydroxyl-containing material contains two or more primary or secondary aliphatic hydroxyl groups (i.e., the hydroxyl group is bonded directly to a non-aromatic carbon atom). The hydroxyl groups can be terminally situated, or they can be pendent from a polymer or copolymer. The molecular weight of the hydroxyl-containing organic material can vary from very low (e.g., 32) to very high (e.g., one million or more). Suitable hydroxyl-containing materials can have low molecular weights (i.e., from 32 to 200), intermediate molecular weights (i.e., from 200 to 10,000, or high molecular weights (i.e., above 10,000). As used herein, all molecular weights are weight average molecular weights.

The hydroxyl-containing materials may be non-aromatic in nature or may contain aromatic functionality. The hydroxyl-containing material may optionally contain heteroatoms in the backbone of the molecule, such as nitrogen, oxygen, sulfur, and the like. The hydroxyl-containing material may, for example, be selected from naturally occurring or synthetically prepared cellulosic materials. The hydroxyl-containing material should be substantially free of groups which may be thermally or photolytically unstable; that is, the material should not decompose or liberate volatile components at temperatures below 100° C. or in the presence of actinic light which may be encountered during the desired photopolymerization conditions for the polymerizable compositions.

Suitable hydroxyl-containing materials useful in the present invention are listed in U.S. Pat. No. 6,187,836 (Oxman et al.).

The hardenable component(s) may also contain hydroxyl groups and cationically active functional groups in a single molecule. An example is a single molecule that includes both hydroxyl groups and epoxy groups.

Glass Ionomers

The hardenable compositions of the present invention may include glass ionomer cements such as conventional glass ionomer cements that typically employ as their main ingredients a homopolymer or copolymer of an ethylenically unsaturated carboxylic acid (e.g., poly acrylic acid, copoly (acrylic, itaconic acid), and the like), a fluoroaluminosilicate (“FAS”) glass, water, and a chelating agent such as tartaric acid. Conventional glass ionomers (i.e., glass ionomer cements) typically are supplied in powder/liquid formulations that are mixed just before use. The mixture will undergo self-hardening in the dark due to an ionic reaction between the acidic repeating units of the polycarboxylic acid and cations leached from the glass.

The glass ionomer cements may also include resin-modified glass ionomer (“RMGI”) cements. Like a conventional glass ionomer, an RMGI cement employs an FAS glass. However, the organic portion of an RMGI is different. In one type of RMGI, the polycarboxylic acid is modified to replace or end-cap some of the acidic repeating units with pendent curable groups and a photoinitiator is added to provide a second cure mechanism, e.g., as described in U.S. Pat. No. 5,130,347 (Mitra). Acrylate or methacrylate groups are usually employed as the pendant curable group. In another type of RMGI, the cement includes a polycarboxylic acid, an acrylate or methacrylate-functional monomer and a photoinitiator, e.g., as in Mathis et al., “Properties of a New Glass Ionomer/Composite Resin Hybrid Restorative”, Abstract No. 51, J. Dent Res., 66:113 (1987) and as in U.S. Pat. No. 5,063,257 (Akahane et al.), U.S. Pat. No. 5,520,725 (Kato et al.), U.S. Pat. No. 5,859,089 (Qian), U.S. Pat. No. 5,925,715 (Mitra) and U.S. Pat. No. 5,962,550 (Akahane et al.). In another type of RMGI, the cement may include a polycarboxylic acid, an acrylate or methacrylate-functional monomer, and a redox or other chemical cure system, e.g., as described in U.S. Pat. No. 5,154,762 (Mitra et al.), U.S. Pat. No. 5,520,725 (Kato et al.), and U.S. Pat. No. 5,871,360 (Kato). In another type of RMGI, the cement may include various monomer-containing or resin-containing components as described in U.S. Pat. No. 4,872,936 (Engelbrecht), U.S. Pat. No. 5,227,413 (Mitra), U.S. Pat. No. 5,367,002 (Huang et al.), and U.S. Pat. No. 5,965,632 (Orlowski). RMGI cements are preferably formulated as powder/liquid or paste/paste systems, and contain water as mixed and applied. The compositions are able to harden in the dark due to the ionic reaction between the acidic repeating units of the polycarboxylic acid and cations leached from the glass, and commercial RMGI products typically also cure on exposure of the cement to light from a dental curing lamp. RMGI cements that contain a redox cure system and that can be cured in the dark without the use of actinic radiation are described in U.S. Pat. No. 6,765,038 (Mitra).

Polyethers or Polysiloxanes (i.e., Silicones)

Dental impression materials are typically based on polyether or polysiloxane (i.e. silicone) chemistry. Polyether materials typically consist of a two-part system that includes a base component (e.g., a polyether with ethylene imine rings as terminal groups) and a catalyst (or accelerator) component (e.g., an aryl sulfonate as a cross-linking agent). Polysiloxane materials also typically consist of a two-part system that includes a base component (e.g., a polysiloxane, such as a dimethylpolysiloxane, of low to moderately low molecular weight) and a catalyst (or accelerator) component (e.g., a low to moderately low molecular weight polymer with vinyl terminal groups and chloroplatinic acid catalyst in the case of addition silicones; or a liquid that consists of stannous octanoate suspension and an alkyl silicate in the case of condensation silicones). Both systems also typically contain a filler, a plasticizer, a thickening agent, a coloring agent, or mixtures thereof. Exemplary polyether impression materials include those described in, for example, U.S. Pat. No. 6,127,449 (Bissinger et al.); U.S. Pat. No. 6,395,801 (Bissinger et al.); and U.S. Pat. No. 5,569,691 (Guggenberger et al.). Exemplary polysiloxane impression materials and related polysiloxane chemistry are described in, for example, U.S. Pat. No. 6,121,362 (Wanek et al.) and U.S. Pat. No. U.S. Pat. No. 6,566,413 Weinmann et al.), and EP Pat. Publication No. 1 475 069 A (Bissinger et al.).

Examples of commercial polyether and polysiloxane impression materials include, but are not limited to, IMPREGUM Polyether Materials, PERMADYNE Polyether Materials, EXPRESS Vinyl Polysiloxane Materials, DIMENSION Vinyl Polysiloxane Materials, and IMPRINT Vinyl Polysiloxane Materials; all available from 3M ESPE (St. Paul, Minn.). Other exemplary polyether, polysiloxane (silicones), and polysulfide impression materials are discussed in the following reference: Restorative Dental Materials, Tenth Edition, edited by Robert G. Craig and Marcus L. Ward, Mosby-Year Book, Inc., St. Louis, Mo., Chapter 11 (Impression Materials).

Photoinitiator Systems

In certain embodiments, the compositions of the present invention are photopolymerizable, i.e., the compositions contain a photopolymerizable component and a photoinitiator (i.e., a photoinitiator system) that upon irradiation with actinic radiation initiates the polymerization (or hardening) of the composition. Such photopolymerizable compositions can be free radically polymerizable or cationically polymerizable.

Suitable photoinitiators (i.e., photoinitiator systems that include one or more compounds) for polymerizing free radically photopolymerizable compositions include binary and tertiary systems. Typical tertiary photoinitiators include an iodonium salt, a photosensitizer, and an electron donor compound as described in U.S. Pat. No. 5,545,676 (Palazzotto et al.). Preferred iodonium salts are the diaryl iodonium salts, e.g., diphenyliodonium chloride, diphenyliodonium hexafluorophosphate, diphenyliodonium tetrafluoroborate, and tolylcumyliodonium tetrakis(pentafluorophenyl)borate. Preferred photosensitizers are monoketones and diketones that absorb some light within a range of 400 nm to 520 nm (preferably, 450 nm to 500 nm). More preferred compounds are alpha diketones that have some light absorption within a range of 400 nm to 520 nm (even more preferably, 450 to 500 nm). Preferred compounds are camphorquinone, benzil, furil, 3,3,6,6-tetramethylcyclohexanedione, phenanthraquinone, 1-phenyl-1,2-propanedione and other 1-aryl-2-alkyl-1,2-ethanediones, and cyclic alpha diketones. Most preferred is camphorquinone. Preferred electron donor compounds include substituted amines, e.g., ethyl dimethylaminobenzoate. Other suitable tertiary photoinitiator systems useful for photopolymerizing cationically polymerizable resins are described, for example, in U.S. Pat. No. 6,765,036 (Dede et al.).

Other suitable photoinitiators for polymerizing free radically photopolymerizable compositions include the class of phosphine oxides that typically have a functional wavelength range of 380 nm to 1200 nm. Preferred phosphine oxide free radical initiators with a functional wavelength range of 380 nm to 450 nm are acyl and bisacyl phosphine oxides such as those described in U.S. Pat. No. 4,298,738 (Lechtken et al.), U.S. Pat. No. 4,324,744 (Lechtken et al.), U.S. Pat. No. 4,385,109 (Lechtken et al.), U.S. Pat. No. 4,710,523 (Lechtken et al.), and U.S. Pat. No. 4,737,593 (Ellrich et al.), U.S. Pat. No. 6,251,963 (Kohler et al.); and EP Application No. 0 173 567 A2 (Ying).

Commercially available phosphine oxide photoinitiators capable of free-radical initiation when irradiated at wavelength ranges of greater than 380 nm to 450 nm include bis(2,4,6-trimethylbenzoyl)phenyl phosphine oxide (IRGACURE 819, Ciba Specialty Chemicals, Tarrytown, N.Y.), bis(2,6-dimethoxybenzoyl)-(2,4,4-trimethylpentyl) phosphine oxide (CGI 403, Ciba Specialty Chemicals), a 25:75 mixture, by weight, of bis(2,6-dimethoxybenzoyl)-2,4,4-trimethylpentyl phosphine oxide and 2-hydroxy-2-methyl-1-phenylpropan-1-one (IRGACURE 1700, Ciba Specialty Chemicals), a 1:1 mixture, by weight, of bis(2,4,6-trimethylbenzoyl)phenyl phosphine oxide and 2-hydroxy-2-methyl-1-phenylpropane-1-one (DAROCUR 4265, Ciba Specialty Chemicals), and ethyl 2,4,6-trimethylbenzylphenyl phosphinate (LUCIRIN LR8893X, BASF Corp., Charlotte, N.C.).

Typically, the phosphine oxide initiator is present in the photopolymerizable composition in catalytically effective amounts, such as from 0.1 weight percent to 5.0 weight percent, based on the total weight of the composition.

Tertiary amine reducing agents may be used in combination with an acylphosphine oxide. Illustrative tertiary amines useful in the invention include ethyl 4-(N,N-dimethylamino)benzoate and N,N-dimethylaminoethyl methacrylate. When present, the amine reducing agent is present in the photopolymerizable composition in an amount from 0.1 weight percent to 5.0 weight percent, based on the total weight of the composition. Useful amounts of other initiators are well known to those of skill in the art.

Suitable photoinitiators for polymerizing cationically photopolymerizable compositions include binary and tertiary systems. Typical tertiary photoinitiators include an iodonium salt, a photosensitizer, and an electron donor compound as described in EP 0 897 710 (Weinmann et al.); in U.S. Pat. No. 5,856,373 (Kaisaki et al.), U.S. Pat. No. 6,084,004 (Weinmann et al.), U.S. Pat. No. 6,187,833 (Oxman et al.), and U.S. Pat. No. 6,187,836 (Oxman et al.); and in U.S. Pat. No. 6,765,036 (Dede et al.). The compositions of the invention can include one or more anthracene-based compounds as electron donors. In some embodiments, the compositions comprise multiple substituted anthracene compounds or a combination of a substituted anthracene compound with unsubstituted anthracene. The combination of these mixed-anthracene electron donors as part of a photoinitiator system provides significantly enhanced cure depth and cure speed and temperature insensitivity when compared to comparable single-donor photoinitiator systems in the same matrix. Such compositions with anthracene-based electron donors are described in U.S. Ser. No. 10/719,598 (Oxman et al.; filed Nov. 21, 2003).

Suitable iodonium salts include tolylcumyliodonium tetrakis(pentafluorophenyl)borate, tolylcumyliodonium tetrakis(3,5-bis(trifluoromethyl)-phenyl)borate, and the diaryl iodonium salts, e.g., diphenyliodonium chloride, diphenyliodonium hexafluorophosphate, diphenyliodonium hexafluoroantimonate, and diphenyliodonium tetrafluoroboarate. Suitable photosensitizers are monoketones and diketones that absorb some light within a range of 450 nm to 520 nm (preferably, 450 nm to 500 nm). More suitable compounds are alpha diketones that have some light absorption within a range of 450 nm to 520 nm (even more preferably, 450 nm to 500 nm). Preferred compounds are camphorquinone, benzil, furil, 3,3,6,6-tetramethylcyclohexanedione, phenanthraquinone and other cyclic alpha diketones. Most preferred is camphorquinone. Suitable electron donor compounds include substituted amines, e.g., ethyl 4-(dimethylamino)benzoate and 2-butoxyethyl 4-(dimethylamino)benzoate; and polycondensed aromatic compounds (e.g. anthracene).

The initiator system is present in an amount sufficient to provide the desired rate of hardening (e.g., polymerizing and/or crosslinking). For a photoinitiator, this amount will be dependent in part on the light source, the thickness of the layer to be exposed to radiant energy, and the extinction coefficient of the photoinitiator. Preferably, the initiator system is present in a total amount of at least 0.01 wt-%, more preferably, at least 0.03 wt-%, and most preferably, at least 0.05 wt-%, based on the weight of the composition. Preferably, the initiator system is present in a total amount of no more than 10 wt-%, more preferably, no more than 5 wt-%, and most preferably, no more than 2.5 wt-%, based on the weight of the composition.

Redox Initiator Systems

In certain embodiments, the compositions of the present invention are chemically hardenable, i.e., the compositions contain a chemically hardenable component and a chemical initiator (i.e., initiator system) that can polymerize, cure, or otherwise harden the composition without dependence on irradiation with actinic radiation. Such chemically hardenable compositions are sometimes referred to as “self-cure” compositions and may include glass ionomer cements, resin-modified glass ionomer cements, redox cure systems, and combinations thereof.

The chemically hardenable compositions may include redox cure systems that include a hardenable component (e.g., an ethylenically unsaturated polymerizable component) and redox agents that include an oxidizing agent and a reducing agent. Suitable hardenable components, redox agents, optional acid-functional components, and optional fillers that are useful in the present invention are described in U.S. Pat. Publication Nos. 2003/0166740 (Mitra et al.) and 2003/0195273 (Mitra et al.).

The reducing and oxidizing agents should react with or otherwise cooperate with one another to produce free-radicals capable of initiating polymerization of the resin system (e.g., the ethylenically unsaturated component). This type of cure is a dark reaction, that is, it is not dependent on the presence of light and can proceed in the absence of light. The reducing and oxidizing agents are preferably sufficiently shelf-stable and free of undesirable colorization to permit their storage and use under typical dental conditions. They should be sufficiently miscible with the resin system (and preferably water-soluble) to permit ready dissolution in (and discourage separation from) the other components of the hardenable composition.

Useful reducing agents include ascorbic acid, ascorbic acid derivatives, and metal complexed ascorbic acid compounds as described in U.S. Pat. No. 5,501,727 (Wang et al.); amines, especially tertiary amines, such as 4-tert-butyl dimethylaniline; aromatic sulfinic salts, such as p-toluenesulfinic salts and benzenesulfinic salts; thioureas, such as 1-ethyl-2-thiourea, tetraethyl thiourea, tetramethyl thiourea, 1,1-dibutyl thiourea, and 1,3-dibutyl thiourea; and mixtures thereof. Other secondary reducing agents may include cobalt (II) chloride, ferrous chloride, ferrous sulfate, hydrazine, hydroxylamine (depending on the choice of oxidizing agent), salts of a dithionite or sulfite anion, and mixtures thereof. Preferably, the reducing agent is an amine.

Suitable oxidizing agents will also be familiar to those skilled in the art, and include but are not limited to persulfuric acid and salts thereof, such as sodium, potassium, ammonium, cesium, and alkyl ammonium salts. Additional oxidizing agents include peroxides such as benzoyl peroxides, hydroperoxides such as cumyl hydroperoxide, t-butyl hydroperoxide, and amyl hydroperoxide, as well as salts of transition metals such as cobalt (III) chloride and ferric chloride, cerium (IV) sulfate, perboric acid and salts thereof, permanganic acid and salts thereof, perphosphoric acid and salts thereof, and mixtures thereof.

It may be desirable to use more than one oxidizing agent or more than one reducing agent. Small quantities of transition metal compounds may also be added to accelerate the rate of redox cure. In some embodiments it may be preferred to include a secondary ionic salt to enhance the stability of the polymerizable composition as described in U.S. Pat. Publication No. 2003/0195273 (Mitra et al.).

The reducing and oxidizing agents are present in amounts sufficient to permit an adequate free-radical reaction rate. This can be evaluated by combining all of the ingredients of the hardenable composition except for the optional filler, and observing whether or not a hardened mass is obtained.

Preferably, the reducing agent is present in an amount of at least 0.01% by weight, and more preferably at least 0.1% by weight, based on the total weight (including water) of the components of the hardenable composition. Preferably, the reducing agent is present in an amount of no greater than 10% by weight, and more preferably no greater than 5% by weight, based on the total weight (including water) of the components of the hardenable composition.

Preferably, the oxidizing agent is present in an amount of at least 0.01% by weight, and more preferably at least 0.10% by weight, based on the total weight (including water) of the components of the hardenable composition. Preferably, the oxidizing agent is present in an amount of no greater than 10% by weight, and more preferably no greater than 5% by weight, based on the total weight (including water) of the components of the hardenable composition.

The reducing or oxidizing agents can be microencapsulated as described in U.S. Pat. No. 5,154,762 (Mitra et al.). This will generally enhance shelf stability of the hardenable composition, and if necessary permit packaging the reducing and oxidizing agents together. For example, through appropriate selection of an encapsulant, the oxidizing and reducing agents can be combined with an acid-functional component and optional filler and kept in a storage-stable state. Likewise, through appropriate selection of a water-insoluble encapsulant, the reducing and oxidizing agents can be combined with an FAS glass and water and maintained in a storage-stable state.

A redox cure system can be combined with other cure systems, e.g., with a hardenable composition such as described U.S. Pat. No. 5,154,762 (Mitra et al.).

Fillers

The compositions of the present invention can optionally contain fillers. Fillers may be selected from one or more of a wide variety of materials suitable for incorporation in compositions used for dental applications, such as fillers currently used in dental restorative compositions, and the like.

The filler is preferably finely divided. The filler can have a unimodial or polymodial (e.g., bimodal) particle size distribution. Preferably, the maximum particle size (the largest dimension of a particle, typically, the diameter) of the filler is less than 20 micrometers, more preferably less than 10 micrometers, and most preferably less than 5 micrometers. Preferably, the average particle size of the filler is less than 0.1 micrometers, and more preferably less than 0.075 micrometer.

The filler can be an inorganic material. It can also be a crosslinked organic material that is insoluble in the resin system (i.e., the hardenable components), and is optionally filled with inorganic filler. The filler should in any event be nontoxic and suitable for use in the mouth. The filler can be radiopaque or radiolucent. The filler typically is substantially insoluble in water.

Examples of suitable inorganic fillers are naturally occurring or synthetic materials including, but not limited to: quartz (i.e., silica, SiO₂); nitrides (e.g., silicon nitride); glasses and fillers derived from, for example, Zr, Sr, Ce, Sb, Sn, Ba, Zn, and Al; feldspar; borosilicate glass; kaolin; talc; zirconia; titania; low Mohs hardness fillers such as those described in U.S. Pat. No. 4,695,251 (Randklev); and submicron silica particles (e.g., pyrogenic silicas such as those available under the trade designations AEROSIL, including “OX 50,” “130,” “150” and “200” silicas from Degussa Corp., Akron, Ohio, and CAB-O-SIL M5 silica from Cabot Corp., Tuscola, Ill.). Examples of suitable organic filler particles include filled or unfilled pulverized polycarbonates, polyepoxides, and the like.

Preferred non-acid-reactive filler particles are quartz (i.e., silica), submicron silica, zirconia, submicron zirconia, and non-vitreous microparticles of the type described in U.S. Pat. No. 4,503,169 (Randklev). Mixtures of these non-acid-reactive fillers are also contemplated, as well as combination fillers made from organic and inorganic materials.

The filler can also be an acid-reactive filler. Suitable acid-reactive fillers include metal oxides, glasses, and metal salts. Typical metal oxides include barium oxide, calcium oxide, magnesium oxide, and zinc oxide. Typical glasses include borate glasses, phosphate glasses, and fluoroaluminosilicate (“FAS”) glasses. FAS glasses are particularly preferred. The FAS glass typically contains sufficient elutable cations so that a hardened dental composition will form when the glass is mixed with the components of the hardenable composition. The glass also typically contains sufficient elutable fluoride ions so that the hardened composition will have cariostatic properties. The glass can be made from a melt containing fluoride, alumina, and other glass-forming ingredients using techniques familiar to those skilled in the FAS glassmaking art. The FAS glass typically is in the form of particles that are sufficiently finely divided so that they can conveniently be mixed with the other cement components and will perform well when the resulting mixture is used in the mouth.

Generally, the average particle size (typically, diameter) for the FAS glass is no greater than 12 micrometers, typically no greater than 10 micrometers, and more typically no greater than 5 micrometers as measured using, for example, a sedimentation analyzer. Suitable FAS glasses will be familiar to those skilled in the art, and are available from a wide variety of commercial sources, and many are found in currently available glass ionomer cements such as those commercially available under the trade designations VITREMER, VITREBOND, RELY X LUTING CEMENT, RELY X LUTING PLUS CEMENT, PHOTAC-FIL QUICK, KETAC-MOLAR, and KETAC-FIL PLUS (3M ESPE Dental Products, St. Paul, Minn.), FUJI II LC and FUJI IX (G-C Dental Industrial Corp., Tokyo, Japan) and CHEMFIL Superior (Dentsply International, York, Pa.). Mixtures of fillers can be used if desired.

The surface of the filler particles can also be treated with a coupling agent in order to enhance the bond between the filler and the resin. The use of suitable coupling agents include gamma-methacryloxypropyltrimethoxysilane, gamma-mercaptopropyltriethoxysilane, gamma-aminopropyltrimethoxysilane, and the like. Silane-treated zirconia-silica (ZrO₂—SiO₂) filler, silane-treated silica filler, silane-treated zirconia filler, and combinations thereof are especially preferred in certain embodiments.

Other suitable fillers are disclosed in U.S. Pat. No. 6,387,981 (Zhang et al.) and U.S. Pat. No. 6,572,693 (Wu et al.) as well as International Application Publication Nos. WO 01/30305 (Zhang et al.), WO 01/30306 (Windisch et al.), WO 01/30307 (Zhang et al.), and WO 03/063804 (Wu et al.). Filler components described in these references include nanosized silica particles, nanosized metal oxide particles, and combinations thereof. Nanofillers are also described in U.S. pat. application Ser. No. 10/847,781 (Kangas et al.); U.S. Ser. No. 10/847,782 (Kolb et al.); U.S. Ser. No. 10/847,803 (Craig et al.); and U.S. Ser. No. 10/847,805 (Budd et al.) all four of which were filed on May 17, 2004. These applications, in summary, describe the following nanofiller containing compositions.

U.S. pat. application Ser. No. 10/847,781 (Kangas et al.) describes stable ionomer compositions (e.g., glass ionomer) containing nanofillers that provide the compositions with improved properties over previous ionomer compositions. In one embodiment, the composition is a hardenable dental composition comprising a polyacid (e.g., a polymer having a plurality of acidic repeating groups); an acid-reactive filler; at least 10 percent by weight nanofiller or a combination of nanofillers each having an average particle size no more than 200 nanometers; water; and optionally a polymerizable component (e.g., an ethylenically unsaturated compound, optionally with acid functionality).

U.S. pat. application Ser. No. 10/847,782 (Kolb et al.) describes stable ionomer (e.g., glass ionomer) compositions containing nanozirconia fillers that provide the compositions with improved properties, such as ionomer systems that are optically translucent and radiopaque. The nanozirconia is surface modified with silanes to aid in the incorporation of the nanozirconia into ionomer compositions, which generally contain a polyacid that might otherwise interact with the nanozirconia causing coagulation or aggregation resulting in undesired visual opacity. In one aspect, the composition can be a hardenable dental composition including a polyacid; an acid-reactive filler; a nanozirconia filler having a plurality of silane-containing molecules attached onto the outer surface of the zirconia particles; water; and optionally a polymerizable component (e.g., an ethylenically unsaturated compound, optionally with acid functionality).

U.S. pat. application Ser. No. 10/847,803 (Craig et al.) describes stable ionomer compositions (e.g., glass ionomers) containing nanofillers that provide the compositions with enhanced optical translucency. In one embodiment, the composition is a hardenable dental composition including a polyacid (e.g., a polymer having a plurality of acidic repeating groups); an acid-reactive filler; a nanofiller; an optional polymerizable component (e.g., an ethylenically unsaturated compound, optionally with acid functionality); and water. The refractive index of the combined mixture (measured in the hardened state or the unhardened state) of the polyacid, nanofiller, water and optional polymerizable component is generally within 4 percent of the refractive index of the acid-reactive filler, typically within 3 percent thereof, more typically within 1 percent thereof, and even more typically within 0.5 percent thereof.

U.S. pat. application Ser. No. 10/847,805 (Budd et al.) describes dental compositions that can include an acid-reactive nanofiller (i.e., a nanostructured filler) and a hardenable resin (e.g., a polymerizable ethylenically unsaturated compound. The acid-reactive nanofiller can include an oxyfluoride material that is acid-reactive, non-fused, and includes a trivalent metal (e.g., alumina), oxygen, fluorine, an alkaline earth metal, and optionally silicon and/or a heavy metal.

For some embodiments of the present invention that include filler (e.g., dental adhesive compositions), the compositions preferably include at least 1% by weight, more preferably at least 2% by weight, and most preferably at least 5% by weight filler, based on the total weight of the composition. For such embodiments, compositions of the present invention preferably include at most 40% by weight, more preferably at most 20% by weight, and most preferably at most 15% by weight filler, based on the total weight of the composition.

For other embodiments (e.g., where the composition is a dental restorative or an orthodontic adhesive), compositions of the present invention preferably include at least 40% by weight, more preferably at least 45% by weight, and most preferably at least 50% by weight filler, based on the total weight of the composition. For such embodiments, compositions of the present invention preferably include at most 90% by weight, more preferably at most 80% by weight, even more preferably at most 70% by weight filler, and most preferably at most 50% by weight filler, based on the total weight of the composition.

Optional Photobleachable and/or Thermochromic Dyes

In some embodiments, compositions of the present invention preferably have an initial color remarkably different than dental structures. Color is preferably imparted to the composition through the use of a photobleachable or photochromic dye. The composition preferably includes at least 0.001% by weight photobleachable or photochromic dye, and more preferably at least 0.002% by weight photobleachable or photochromic dye, based on the total weight of the composition. The composition preferably includes at most 1% by weight photobleachable or photochromic dye, and more preferably at most 0.1% by weight photobleachable or photochromic dye, based on the total weight of the composition. The amount of photobleachable and/or photochromic dye may vary depending on its extinction coefficient, the ability of the human eye to discern the initial color, and the desired color change. Suitable photobleachable dyes are disclosed, for example, in U.S. Pat. No. 6,670,436 (Burgath et al.).

For embodiments including a photobleachable dye, the color formation and bleaching characteristics of the photobleachable dye varies depending on a variety of factors including, for example, acid strength, dielectric constant, polarity, amount of oxygen, and moisture content in the atmosphere. However, the bleaching properties of the dye can be readily determined by irradiating the composition and evaluating the change in color. Preferably, at least one photobleachable dye is at least partially soluble in a hardenable resin.

Exemplary classes of photobleachable dyes are disclosed, for example, in U.S. Pat. No. 6,331,080 (Cole et al.), U.S. Pat. No. 6,444,725 (Trom et al.), and U.S. Pat. No. 6,528,555 (Nikutowski et al.). Preferred dyes include, for example, Rose Bengal, Methylene Violet, Methylene Blue, Fluorescein, Eosin Yellow, Eosin Y, Ethyl Eosin, Eosin bluish, Eosin B, Erythrosin B, Erythrosin Yellowish Blend, Toluidine Blue, 4′,5′-Dibromofluorescein, and combinations thereof.

The color change in the inventive compositions is initiated by light. Preferably, the composition's color change is initiated using actinic radiation using, for example, a dental curing light which emits visible or near infrared (IR) light for a sufficient amount of time. The mechanism that initiates the color change in the compositions of the invention may be separate from or substantially simultaneous with the hardening mechanism that hardens the resin. For example, a composition may harden when polymerization is initiated chemically (e.g., redox initiation) or thermally, and the color change from an initial color to a final color may occur subsequent to the hardening process upon exposure to actinic radiation.

The change in composition color from an initial color to a final color is preferably quantified by a color test. Using a color test, a value of ΔE* is determined, which indicates the total color change in a 3-dimensional color space. The human eye can detect a color change of approximately 3 ΔE* units in normal lighting conditions. The dental compositions of the present invention are preferably capable of having a color change, ΔE*, of at least 20; more preferably, ΔE* is at least 30; most preferably ΔE* is at least 40.

Optional Acid-Generating Component and Acid-Reactive Component

Optionally, the hardenable dental composition of the present invention can include an acid-generating component and an acid-reactive component as described, for example, in U.S. Patent Application Ser. No. ______, filed the same day herewith (Attorney Docket No. 59962US002 entitled “METHODS FOR REDUCING BOND STRENGTHS, DENTAL COMPOSITIONS, AND THE USE THEREOF”).

Acid-generating components typically include an acid-generating compound, and optionally a sensitizer. Preferably, the acid-generating component generates an acid upon irradiation (i.e., a photo-acid). Typically, the acid can react with greater than a stoichiometric amount of acid-reactive groups. Preferably, dental compositions of the present invention do not include groups that would act to deplete the generated acid in amounts sufficient to interfere with the desired reaction of the generated acid with the acid-reactive component.

Exemplary acid-generating components include iodonium salts (e.g., diaryliodonium salts), sulfonium salts (e.g., triarylsulfonium salts and dialkylphenacylsulfonium salts), selenonium salts (e.g., triarylselenonium salts), sulfoxonium salts (e.g., triarylsulfoxonium salts, aryloxydiarylsulfoxonium salts, and dialkylphenacylsulfoxonium salts), diazonium salts (e.g., aryldiazonium salts), organometallic complex cations (e.g., ferrocenium salts), halo-S-triazenes, trihaloketones, a-sulfonyloxy ketones, silyl benzyl ethers, and combinations thereof. When the acid-generating component is a salt of a cationic species (e.g., an “onium” salt), typical counterions for the salt include, for example, tetrafluoroborate, hexafluorophosphate, hexafluoroarsenate, hexafluoroantimonate, and combinations thereof. Exemplary acid-generating components include those disclosed, for example, in Crivello et al., “Photoinitiators for Free Radical, Cationic and Anionic Photopolymerization,” G. Bradley, Editor, Volume 3, Chapter 6 (1998), and U.S. Pat. No. 6,187,833 (Oxman et al.), U.S. Pat. No. 6,395,124 (Oxman et al.), U.S. Pat. No. 6,765,036 (Dede et al.), U.S. Pat. No. 3,775,113 (Bonham et al.), U.S. Pat. No. 3,779,778 (Smith et al.), U.S. Pat. No. 3,954,475 (Bonham et al.), U.S. Pat. No. 4,329,384 (Vesley et al.), U.S. Pat. No. 4,330,570 (Giuliani et al.), U.S. Pat. No. 5,089,374 (Saeva), and U.S. Pat. No. 5,141,969 (Saeve et al.).

Preferably the acid-generating component includes a sulfonium salt. Exemplary sulfonium salts include, for example, triaryl sulfonium hexafluoroantimonate (Ar₃S⁺ SbF₆⁻, available under the trade designation CYRACURE CPI-6976 from Advanced Research Corporation, Catoosa, Okla.); triaryl sulfonium hexafluorophosphate (Ar₃S⁺ PF₆⁻, 50% solution in propylene carbonate, available under the trade designation CYRACURE CPI-6992, from Aceto Corp., Lake Success, N.Y.); and triaryl sulfonium N-(trifluoromethanesulfonyl)trifluoromethane-sulfonamido anion (Ar₃S⁺ N(SO₂CF₃)₂)⁻, which can be prepared as generally described in U.S. Pat. No. 5,554,664 (Lamanna et al.).

Exemplary sensitizers include anthracene derivatives (e.g., 2-methylanthracene (2-MA, Sigma-Aldrich) and 2-ethyl-9,10-dimethoxyanthracene (EDMOA, Sigma-Aldrich)), perylene, phenothiazene, and other polycyclic aromatic compounds as described, for example, in U.S. Pat. No. 6,765,036 (Dede et al.) and U.S. Pat. Publication No. 2005/0113477 (Oxman et al.), and combinations thereof. One of skill in the art could select, without undue experimentation, an appropriate sensitizer for sensitizing a specific acid-generating component (e.g., a sulfonium salt) based on the principles described, for example, in Crivello et al., “Photoinitiators for Free Radical, Cationic and Anionic Photopolymerization,” G. Bradley, Editor, Volume 3, Chapter 6 (1998). Preferably, a sensitizer can be selected that absorbs at a different wavelength than the photoinitiator; has a singlet or triplet state that is higher in energy than the corresponding singlet or triplet state in the acid-generating component; and/or has an oxidation potential such that reduction of the acid-generating component is energetically favorable. For example, 2-methylanthracene is an appropriate sensitizer for sensitizing triaryl sulfonium hexafluoroantimonate.

As used herein, an “acid-reactive component” refers to a component (typically a compound) that includes one or more acid-reactive groups. As used herein, an “acid-reactive group” refers to a group that undergoes, after reaction with an acid, substantial breaking (e.g., observable by spectroscopic techniques) of chemical bonds within the group to form two or more separate groups, often upon heating to an elevated temperature (i.e., at least 42° C.). Preferably, the elevated temperature is no greater than 200° C., more preferably no greater than 150° C., and even more preferably no greater than 100° C., and most preferably no greater than 80° C. Suitable methods for determining whether substantial breaking of chemical bonds occurs after reaction of a component with an acid would be apparent to one of skill in the art. Suitable methods include, for example, spectroscopic methods such as nuclear magnetic resonance (NMR) spectroscopy (including ¹H, ¹³C, and/or other appropriate nuclei); and ultraviolet (UV), visible, and infrared (IR) spectroscopy, including near IR (NIR) spectroscopy. For example, ¹H and/or ¹³C NMR spectra can be conveniently run in an NMR tube by dissolving the component in a non-acidic solvent (e.g., CDCl₃), adding an acid (e.g., CF₃CO₂D), and observing the disappearance of peaks arising from the component or the appearance of peaks arising from a reaction product at the desired temperature.

Acid-reactive components suitable for use in hardenable dental compositions of the present invention are preferably hardenable components that include one or more acid-reactive groups. Typically, each acid-reactive group is a multivalent group linking a plurality (i.e., two or more) of hardenable groups. In certain embodiments, the hardenable acid-reactive component is an ethylenically unsaturated compound. For example, in such embodiments, the acid-reactive group can be a divalent group linking two ethylenically unsaturated groups.

Acid reactive groups are well known in the art. Such groups include, for example, functionalities typically used in protection methodologies in organic synthesis, where the protecting group can be designed for removal under acidic conditions. See, for example, Greene et al. Protective Groups in Organic Synthesis, Wiley-Interscience (1999); Taylor et al., Chem. Mater., 3:1031-1040 (1991); and U.S. Pat. No. 6,652,970 (Everaerts et al.).

Optional Thermally Labile Components

Optionally, the hardenable dental composition of the present invention can include a thermally labile component as described, for example, in U.S. Pat. Application Ser. No. ______, filed the same day herewith (Attorney Docket No. 60717US002 entitled “DENTAL COMPOSITIONS INCLUDING A THERMALLY LABILE COMPONENT, AND THE USE THEREOF”).

As used herein, a “thermally labile component” refers to a component (typically a compound) that includes one or more thermally labile groups. As used herein, a “thermally labile group” refers to a group that undergoes substantial breaking (e.g., observable by spectroscopic techniques) of chemical bonds within the group to form two or more separate groups upon heating to an elevated temperature (i.e., at least 42° C.). Preferably, the elevated temperature is no greater than 200° C., more preferably no greater than 150° C., and even more preferably no greater than 100° C., and most preferably no greater than 80° C. Suitable methods for determining whether substantial breaking of chemical bonds occurs upon heating a component to an elevated temperature would be apparent to one of skill in the art. Suitable methods include, for example, spectroscopic methods such as nuclear magnetic resonance (NMR) spectroscopy (including ¹H, ¹³C, and/or other appropriate nuclei); and ultraviolet (UV), visible, and infrared (IR) spectroscopy, including near IR (NIR) spectroscopy. For example, ¹H and/or ¹³C NMR spectra can be conveniently run in an NMR tube by dissolving the component in a solvent (e.g., CDCl₃), heating to an elevated temperature, and observing the disappearance of peaks arising from the component or the appearance of peaks arising from a reaction product at the desired temperature.

In certain embodiments, thermally labile components suitable for use in hardenable dental compositions of the present invention are preferably hardenable components that include one or more thermally labile groups. Typically, each thermally labile group is a multivalent group linking a plurality (i.e., two or more) of hardenable groups. In certain embodiments, the hardenable thermally labile component is an ethylenically unsaturated compound. For example, in such embodiments, the thermally labile group can be a divalent group linking two ethylenically unsaturated groups.

Thermally labile groups are well known in the art. Such groups include, for example, oxime esters as disclosed, for example, in U.S. Pat. No. 6,652,970 (Everaerts et al.), and groups including cycloaddition adducts as disclosed, for example, in U.S. Pat. Nos. 6,825,315 (Aubert), U.S. Pat. No. 6,147,141 (Iyer et al.), and PCT International Patent Application Publication No. WO 98/09913 (Rotello).

Optional Radiation-To-Heat Converters

Optionally, the hardenable dental composition of the present invention can include a radiation-to-heat converter as described, for example, in U.S. Pat. Application Ser. No. ______, filed the same day herewith (Attorney Docket No. 60716US002 entitled “DENTAL COMPOSITIONS INCLUDING RADIATION-TO-HEAT CONVERTERS, AND THE USE THEREOF”). Hardened dental compositions that include a radiation-to-heat converter can allow for heating the hardened dental composition by irradiating the composition.

A radiation-to-heat converter is typically a radiation absorber that absorbs incident radiation and converts at least a portion (e.g., at least 50%) of the incident radiation into heat. In some embodiments, the radiation-to-heat converter can absorb light in the infrared, visible, or ultraviolet regions of the electromagnetic spectrum and convert the absorbed radiation into heat. In other embodiments, the radiation-to-heat converter can absorb radio frequency (RF) radiation and convert the absorbed radiation into heat. The radiation absorber(s) are typically highly absorptive of the selected imaging radiation.

A wide variety of radiation-to-heat converters can be used including, for example, organic compounds, inorganic compounds, and metal-organic compounds. Such radiation-to-heat converters can include, for example, dyes (e.g., visible dyes, ultraviolet dyes, infrared dyes, fluorescent dyes, and radiation-polarizing dyes), pigments, metals, metal compounds, metal films, and other suitable absorbing materials. Many classes of organic and metal-organic dyes are described in “Infrared Absorbing Dyes”, edited by Masaru Matsuoka, Plenum Press (New York, 1990). These classes of dyes include azo dyes, pyrazolone azo dyes, methine and cyanine dyes, porphyrin dyes, phthalocyanine dyes, quinine dyes such as anthraquinones and naphthaquinones, pyrylium and squarylium dyes, aminium and diimonium dyes. See, also, U.S. Pat. No. 6,759,177 (Shimada et al.). Radiation-to-heat converters can be selected as desired by one of skill in the art based on properties including, for example, solubility in and/or compatibility with the specific hardenable dental composition or solvent therefore, as well as the wavelength range of absorption. Typically, dyes and/or pigments are preferred for use as radiation-to-heat converters that absorb light in the infrared, visible, or ultraviolet regions of the electromagnetic spectrum.

For some embodiments, near infrared (NIR) absorbing pigments and/or dyes are preferred by use as radiation-to-heat converters to allow for heating by irradiating with NIR radiation. Such NIR absorbing materials typically absorb at wavelengths greater than 750 nanometers, and sometimes at wavelengths greater than 800, 850, or even 900 nanometers. Such NIR absorbing materials typically absorb at wavelengths less than 2000 nanometers, and sometimes at wavelengths less than 1500, 1200, or even 1000 nanometers.

A wide variety of pigments and/or dyes can be used as NIR absorbing radiation-to-heat converters. Useful pigments include, for example, indium tin oxide (ITO), antimony tin oxide (ATO), other tin oxide pigments, lanthanum hexaboride (LAB₆), porphyrin and phthalocyanine pigments, thioindigo pigments, carbon black, azo pigments, quinacridone pigments, nitroso pigments, natural pigments, and azine pigments. Useful dyes include, for example, NIR absorbing cyanine dyes, NIR absorbing azo dyes, NIR absorbing pyrazolone dyes, NIR absorbing phthalocyanine dyes, NIR absorbing anthraquinone and naphthaquinone dyes, nickel or platinum dithiolene complexes, squarilium dyes, carbonium dyes, methine dyes, diimonium dyes, aminium dyes, croconium dyes, quinoneimine dyes, and pyrylium dyes such as those available under the trade designations IR-27 and IR-140 from Sigma-Aldrich (St. Louis, Mo.) or Epolin Inc. (Newark, N.J.).

In some embodiments, the radiation-to-heat converter can be a radio frequency (RF) absorbing magnetic ceramic powder, to allow for heating by irradiating with RF radiation. Exemplary ceramic powders include, for example, NiZn ferrite available under the trade designation FERRITE N23 from National Magnetics Group (Bethlehem, Pa.) with a reported average particle size of 1.0 micrometer and a Curie Temperature (T_{c}) of 95° C.; and Mg—Mn—Zn mixed ferrite available under the trade designation FERRITE R from National Magnetics Group (Bethlehem, Pa.) with a reported average particle size of 1.0 micrometer and a Curie Temperature (T_{c}) of 90° C. Such ceramic powders are capable of absorbing radio frequency (RF) radiation and thereby increasing in temperature. At the reported Curie Temperatures, the ferrites will no longer absorb RF radiation and continue to increase in temperature. Typical RF radiation useful in this invention has an intensity range of 10 μW/cm² to 100 μW/cm² and a frequency of 10 KHz to 10 KHz.

Miscellaneous Optional Additives

Optionally, compositions of the present invention may contain solvents (e.g., alcohols (e.g., propanol, ethanol), ketones (e.g., acetone, methyl ethyl ketone), esters (e.g., ethyl acetate), other nonaqueous solvents (e.g., dimethylformamide, dimethylacetamide, dimethylsulfoxide, 1-methyl-2-pyrrolidinone)), and water.

If desired, the compositions of the invention can contain additives such as indicators, dyes, pigments, inhibitors, accelerators, viscosity modifiers, wetting agents, buffering agents, stabilizers, and other similar ingredients that will be apparent to those skilled in the art. Viscosity modifiers include the thermally responsive viscosity modifiers (such as PLURONIC F-127 and F-108 available from BASF Wyandotte Corporation, Parsippany, N.J.) and may optionally include a polymerizable moiety on the modifier or a polymerizable component different than the modifier. Such thermally responsive viscosity modifiers are described in U.S. Pat. No. 6,669,927 (Trom et al.) and U.S. Pat. Publication No. 2004/0151691 (Oxman et al.).

Additionally, medicaments or other therapeutic substances can be optionally added to the dental compositions. Examples include, but are not limited to, fluoride sources, whitening agents, anticaries agents (e.g., xylitol), calcium sources, phosphorus sources, remineralizing agents (e.g., calcium phosphate compounds), enzymes, breath fresheners, anesthetics, clotting agents, acid neutralizers, chemotherapeutic agents, immune response modifiers, thixotropes, polyols, anti-inflammatory agents, antimicrobial agents (in addition to the antimicrobial lipid component), antifungal agents, agents for treating xerostomia, desensitizers, and the like, of the type often used in dental compositions. Combination of any of the above additives may also be employed. The selection and amount of any one such additive can be selected by one of skill in the art to accomplish the desired result without undue experimentation.

Methods

Hardenable and hardened dental compositions of the present invention (e.g., compositions that in certain embodiments include a thermally responsive additive) can be used for a variety of dental and orthodontic applications that utilize a material capable of adhering (e.g., bonding) to a tooth structure. Preferred uses include applications in which it is desired that the hardened dental composition be removed from the tooth structure at some point in time. Uses for such hardenable and hardened dental compositions include, for example, uses as adhesives (e.g., dental and/or orthodontic adhesives), cements (e.g., glass ionomer cements, resin-modified glass ionomer cements, and orthodontic cements), primers (e.g., orthodontic primers), restoratives, liners, sealants (e.g., orthodontic sealants), coatings, and combinations thereof.

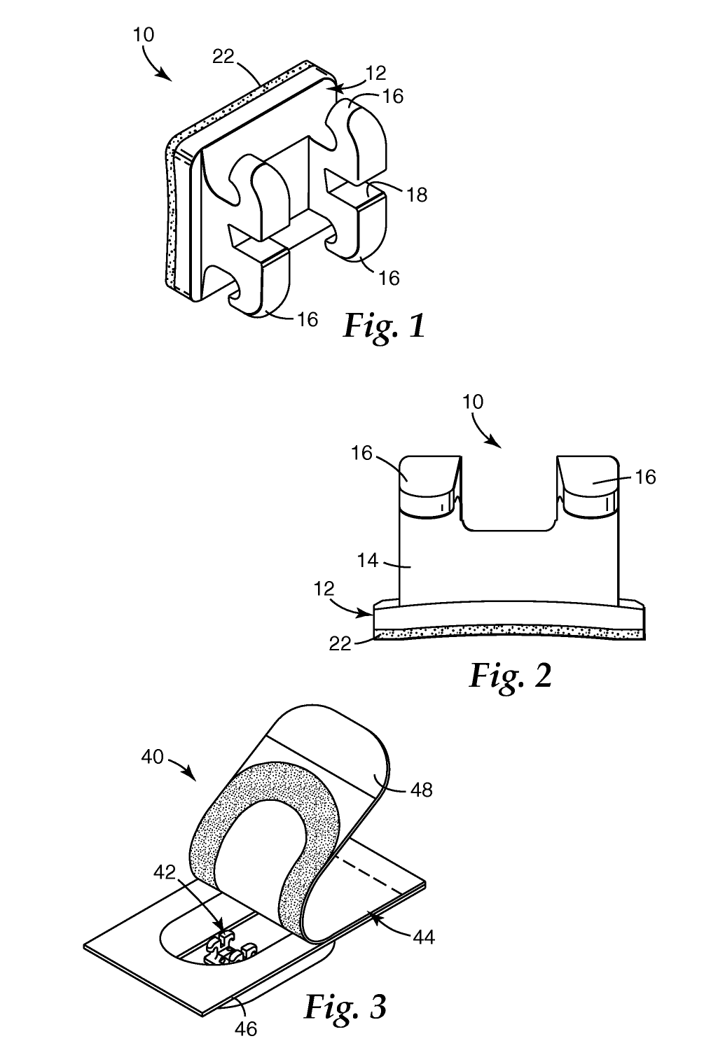

One preferred use for such hardenable or hardened dental compositions includes adhering an orthodontic appliance to a tooth structure. Exemplary embodiments for an orthodontic appliance having a hardenable or hardened dental composition of the present invention on the base thereof are illustrated in FIGS. 1-6. It should be noted that for such embodiments, a practitioner can apply the hardenable dental composition to the base of the orthodontic appliance, and then optionally harden the composition. Alternatively, an orthodontic appliance having a hardenable (or hardened) dental composition on the base thereof can be supplied, for example, by a manufacturer, as a “precoated” orthodontic appliance. In yet other embodiments, a practitioner can apply a hardenable dental composition (e.g., an orthodontic primer) to a tooth structure, optionally harden the composition, and then adhere the orthodontic appliance (typically having a hardenable orthodontic adhesive thereon) to the tooth structure.

In FIGS. 1 and 2, an exemplary orthodontic appliance is designated by the numeral 10 and is a bracket, although other appliances such as buccal tubes, buttons and other attachments are also possible. The appliance 10 includes a base 12. The appliance 10 also has a body 14 that extends outwardly from the base 12. Base 12 can be a flange made of metal, plastic, ceramic, and combinations thereof. Base 12 can include a mesh-like structure, such as a fine wire screen. Base 12 can include particles (such as shards, grit, spheres, or other structure that optionally includes undercuts). Alternatively, the base 12 can be a custom base formed from one or more hardened dental composition layer(s) (e.g., hardened dental compositions of the present invention, hardened orthodontic adhesives, hardened orthodontic primers, or combinations thereof). Tiewings 16 are connected to the body 14, and an archwire slot 18 extends through a space between the tiewings 16. The base 12, the body 14, and tiewings 16 may be made of any one of a number of materials suitable for use in the oral cavity and having sufficient strength to withstand the correction forces applied during treatment. Suitable materials include, for example, metallic materials (such as stainless steel), ceramic materials (such as monocrystalline or polycrystalline alumina), and plastic materials (such as fiber-reinforced polycarbonate). Optionally, the base 12, the body 14, and the tiewings 16 are integrally made as a unitary component.

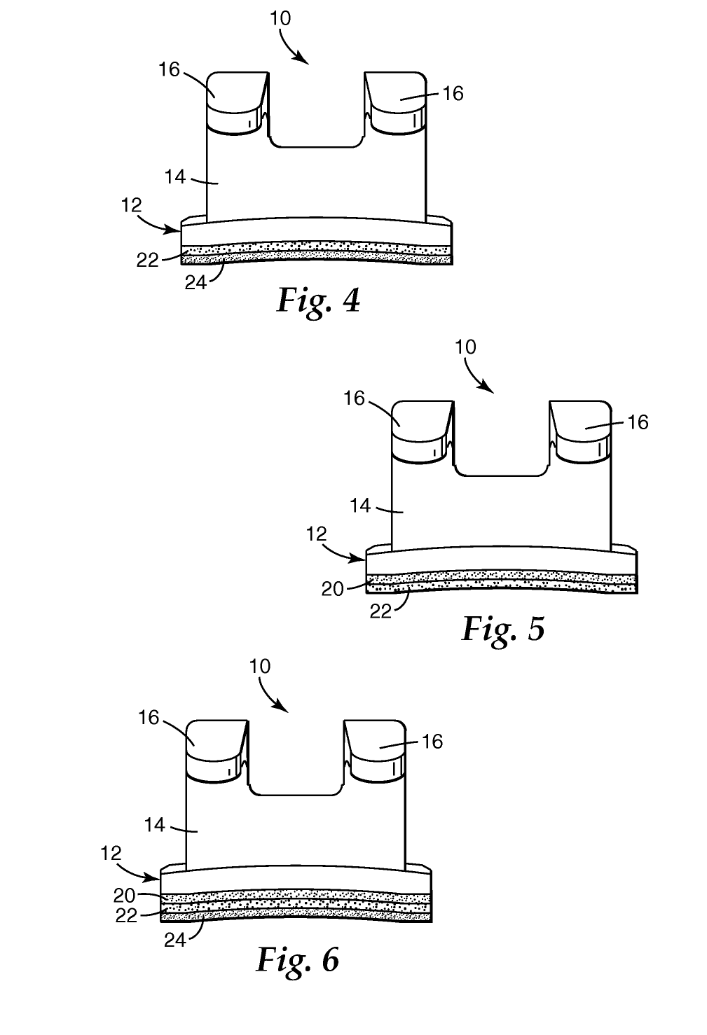

In the exemplary embodiment illustrated in FIGS. 1 and 2, a layer of a hardenable or hardened dental composition of the present invention 22 (hereinafter “composition layer 22”), which is typically an orthodontic adhesive, an orthodontic primer, or an orthodontic sealant, extends across the base 12 of the appliance 10. The composition layer 22 can serve in whole or at least in part to securely fix the appliance 10 to the patient's tooth by a bond having sufficient strength to resist unintended detachment from the tooth during the course of treatment. In one embodiment, the composition layer 22 is applied by the manufacturer to the base 12 of the appliance 10. It should be understood that orthodontic appliance 10 can optionally include additional layer(s) of dental compositions (e.g., orthodontic adhesives, orthodontic primers, or combinations thereof, which are not illustrated in FIGS. 1 and 2) in contact with composition layer 22. Specifically, such additional layer(s) can be between base 12 and composition layer 22; on composition layer 22 opposite base 12; or both. Such layers may or may not cover the same area, and may independently be discontinuous (e.g., a patterned layer) or continuous (e.g., non-patterned) materials extending across all or a portion of adhesive 22. Exemplary appliances including such additional layer(s) are illustrated in FIGS. 4-6.

Orthodontic appliances including multiple hardenable or hardened dental composition layers as described herein can be prepared by methods known to one of skill in the art. Suitable methods include, for example, applying, dispensing, or printing the layers of composition on an appliance or a substrate. Multiple layers may be applied simultaneously or sequentially.

A useful method for applying multiple layers of hardenable dental composition(s) on an orthodontic appliance or a substrate includes, for example, using automated fluid dispensing systems such as those available under the trade designation AUTOMOVE from Asymtek (Carlsbad, Calif.). Such automated fluid dispensing systems are useful for dispensing both patterned and non-patterned layers. Other useful systems include, for example, piston dispensing systems and multiple resolution fluid applicators as described, for example, in U.S. Pat. No. 6,513,897 (Tokie) and U.S. Pat. Application Publication No. 2005/0136370 A1 (Brennan et al.).

Once the hardenable dental composition layer(s) have been applied to an orthodontic appliance or a substrate, the appliance or substrate can conveniently be packaged in a container. Exemplary containers are well known in the art and are disclosed, for example, in U.S. Pat. No. 5,172,809 (Jacobs et al.) and U.S. Pat. No. 6,089,861 (Kelly et al.).

Referring to FIG. 3, an exemplary embodiment of packaged article 40 including orthodontic appliance 42 having hardenable dental composition layer(s) coated on the base thereof is shown. Package 44 includes container 46 and cover 48. Cover 48, which is releasably connected to container 46 as initially provided, is peeled from container 46 to open the package for removal of orthodontic appliance 42. In FIG. 3, cover 48 has been peeled back from container 46 to partially open package 44.

In preferred embodiments, the package provides excellent protection against degradation of the hardenable dental composition(s) (e.g., photocurable materials), even after extended periods of time. Such containers are particularly useful for protecting dyes that impart a color changing feature to the adhesive. Such containers preferably effectively block the passage of actinic radiation over a broad spectral range, and as a result, the compositions do not prematurely lose color during storage.

In preferred embodiments, the package includes container 46 comprising a polymer and metallic particles. As an example, container 46 may be made of polypropylene that is compounded with aluminum filler or receives an aluminum powder coating as disclosed, for example, in U.S. Pat. Application Publication No. 2003/0196914 A1 (Tzou et al.). The combination of polymer and metallic particles provides a highly effective block to the passage of actinic radiation to color changing dyes, even though such dyes are known to be highly sensitive to light. Such containers also exhibit good vapor barrier properties. As a result, the rheological characteristics of the hardenable dental composition(s) are less likely to change over extended periods of time. For example, the improved vapor barrier properties of such containers provide substantial protection against degradation of the handling characteristics of adhesives so that the compositions do not prematurely cure or dry or become otherwise unsatisfactory. Suitable covers 48 for such containers can be made of any material that is substantially opaque to the transmission of actinic radiation so that the compositions do not prematurely cure. Examples of suitable materials for cover 48 include laminates of aluminum foil and polymers. For example, the laminate may comprise a layer of polyethyleneterephthalate, adhesive, aluminum foil, adhesive and oriented polypropylene.

In some embodiments, a packaged orthodontic appliance including a hardenable dental composition of the present invention thereon may include a release substrate as described, for example, in U.S. Pat. No. 6,183,249 (Brennan et al.).

In other embodiments, a packaged orthodontic appliance including a hardenable dental composition of the present invention thereon may not include a release substrate. In one embodiment, the package includes a substrate with at least one recess with an interior surface. The package includes a means for positioning the orthodontic appliance inside the recess such that the composition layer(s) do not separate from the appliance upon removal of the appliance from the recess. Preferably, the package further includes a cover for the recess and a means for maintaining the cover in contact with the substrate, wherein the means for positioning the orthodontic appliance includes means suspending the appliance in the recess such that the composition layer(s) do not contact the interior surface of the recess. Such packages are disclosed, for example, in U.S. Pat. No. 5,172,809 (Jacobs et al.).

In another embodiment the orthodontic appliance has a base for bonding the appliance to a tooth structure and a body extending from the base and at least two opposed tiewings extending away from the body. The base and at least one of the tiewings extend past the body in a gingival direction and present a gingival recess. The base and at least one other of the tiewings extend past the body in an occlusal direction and present an occlusal recess. The package includes a carrier having a pair of arms extending toward each other. Each of the arms has an outer end section, with the outer end sections being spaced apart from each other and presenting a channel therebetween. The orthodontic appliance is located in the channel and is supported by the arms with one of the outer end sections extending into the occlusal recess and the other of the outer end sections extending into the gingival recess. Such orthodontic appliances and packages are described, for example, in U.S. Pat. No. 6,089,861 (Kelly et al.).

In some embodiments, a packaged article can include a set of orthodontic appliances, wherein at least one of the appliances has a hardenable dental composition of the present invention thereon. Additional examples of articles and sets of appliances are described in U.S. Pat. Application Publication No. 2005/0133384 A1 (Cinader et al.). Packaged orthodontic appliances are described, for example, in U.S. Pat. Application Publication No. 2003/0196914 A1 (Tzou et al.) and U.S. Pat. No. 4,978,007 (Jacobs et al.), U.S. Pat. No. 5,015,180 (Randklev), U.S. Pat. No. 5,328,363 (Chester et al.), and U.S. Pat. No. 6,183,249 (Brennan et al.).

An orthodontic appliance having a hardenable dental composition of the present on the base thereof may be bonded to a tooth structure using methods (e.g., direct or indirect bonding methods) that are well known in the art. Upon application of the orthodontic appliance to the tooth structure, the hardenable dental composition of the present invention can be hardened to adhere the orthodontic appliance to the tooth structure. A variety of suitable methods of hardening the composition are known in the art. For example, in some embodiments the hardenable dental composition can be hardened by exposure to UV or visible light. In other embodiments, the hardenable dental composition can be provided as a multi-part composition that hardens upon combining the two or more parts.

When desired, typically upon completion of the orthodontic treatment process, the practitioner needs to remove the orthodontic appliance from the tooth structure. Hardened dental compositions of the present invention are designed to reduce the bond strength upon heating to allow for convenient removal of not only the orthodontic appliance, but also for removal of any hardened dental composition remaining on the tooth structure after removal of the appliance.

The hardened dental composition can be heated by any convenient method including, but not limited to, heating with lasers, warm water, electrothermal debonding units, heated gel tray, as well as other methods known in the art.

Alternatively, for hardened dental compositions including radiation-to-heat converters, the hardened dental composition can optionally be heated by irradiation with radiation that is absorbed by the radiation-to-heat converter. A wide variety of radiation sources can be used including, for example, lasers, laser diodes, quartz-tungsten-halogen lamps, mercury lamps, doped mercury lamps, deuterium lamps, plasma arc lamps, LED sources, and other sources known in the art.

The hardened dental composition can optionally be heated to a convenient temperature for a time sufficient to decrease the bond strength and allow for convenient removal of the orthodontic appliance from the tooth structure. Preferably the temperature and time are chosen to prevent damage to the tooth structure as described, for example, in Zach et al. in “Endodontics,” Bender, Editor, pp. 515-530 (1965). See, also, Laufer et al., Journal of Biomechanical Engineering, 107:234-239 (1985); Launay et al., Lasers in Surgery and Medicine, 7:473-477(1987); Azzeh et al., American Journal of Orthodontics and Dentofacial Orthopedics, 123:79-83 (2003); and Uysal et al., Angle Orthodontist, 75:220-225 (2005). Typically, by using heating techniques that rapidly heat the dental composition, higher temperatures can be used for shorter durations without damaging the tooth structure.

In certain embodiments, at least a portion, and preferably all, of the hardened dental composition, optionally, is heated to at least 42° C., sometimes at least 50° C., and other times at least 70° C. Typically, the hardened dental composition is heated to at most 200° C., sometimes at most 150° C., other times to at most 100° C., and even other times to at most 80° C. The selected temperature is maintained for a time sufficient to result in the desired decrease in bond strength. In certain embodiments, the time is at most 10 minutes, sometimes at most 10 seconds, and other times at most 1 second. The decrease in bond strength typically results in fracture within the hardened composition layer.

In some embodiments, the orthodontic appliance includes an additional dental composition layer. Such additional dental composition layers can include, for example, unhardened or hardened dental compositions (e.g., in certain embodiments, a conventional dental composition not including a thermally responsive additive). The inclusion of additional layers can influence, for example, where fracture takes place during debonding of the orthodontic appliance from the tooth structure, as described herein below.

For example, FIG. 4 illustrates an embodiment in which orthodontic appliance 10 has one additional dental composition layer 24 in contact with composition layer 22. Composition layer 22 may be either an unhardened or hardened dental composition of the present invention. Additional layer 24 is on composition layer 22 opposite base 12. Additional layer 24 is typically an unhardened dental composition (e.g., an orthodontic adhesive, an orthodontic primer, or a combination thereof). Upon application of orthodontic appliance 10 to a tooth structure, additional layer 24 (and composition layer 22 if not already hardened) can be hardened by a variety of methods as described herein above to adhere the orthodontic appliance to the tooth structure.

In some embodiments, additional layer 24 can be a hardenable orthodontic primer that is coated on the tooth structure (and optionally hardened) before the orthodontic appliance with composition layer 22 thereon is adhered to the tooth surface.

Upon completion of the orthodontic treatment, at least a portion of hardened composition layer 22 can be heated to reduce the bond strength, and preferably allow fracture within heated composition layer 22 upon removal of the orthodontic appliance. Fracture within heated composition layer 22 results in fracture near the orthodontic appliance and away from the tooth structure. Further, the heated, hardened composition layer 22 (e.g., an orthodontic adhesive) typically has a lower modulus, and therefore is softer to allow for easier cleanup and/or removal of any remnants of the hardened composition. Therefore, after orthodontic treatment, one embodiment of FIG. 4 would be where composition layer 22 and additional layer 24 are both hardened orthodontic adhesives. In another embodiment, composition layer 22 would be a hardened orthodontic adhesive and additional layer 24 would be a hardened orthodontic primer.

FIG. 5 illustrates another embodiment in which orthodontic appliance 10 has one additional dental composition layer 20 in contact with composition layer 22. Additional layer 20 is between base 12 and composition layer 22. Additional layer 20 is typically an unhardened or hardened dental composition (e.g., an orthodontic adhesive, an orthodontic primer, or a combination thereof). Composition layer 22 is typically unhardened. Upon application of orthodontic appliance 10 to a tooth structure, composition layer 22 (and additional layer 20 if not already hardened) can be hardened by a variety of methods as described herein above to adhere the orthodontic appliance to the tooth structure.

In some embodiments, composition layer 22 can be a hardenable orthodontic primer that is coated on the tooth structure (and optionally hardened) before the orthodontic appliance with additional layer 20 thereon is adhered to the tooth surface.

Upon completion of the orthodontic treatment, at least a portion of hardened composition layer 22 can be heated to reduce the bond strength, and preferably allow fracture within heated composition layer 22 upon removal of the orthodontic appliance. Fracture within heated composition layer 22 results in fracture near the tooth structure. For embodiments in which composition layer 22 is an orthodontic primer and additional layer 20 is an orthodontic adhesive, the hardened orthodontic adhesive is substantially retained on the removed orthodontic appliance. As used herein, “substantially retained on the removed orthodontic appliance” means that at least 50% by weight, and preferably at least 75% by weight of the orthodontic adhesive is retained on the removed orthodontic appliance. When the hardened orthodontic adhesive is substantially retained on the removed orthodontic appliance, clean up and removal of any adhesive remaining on the tooth structure is more convenient, because less adhesive remains on the tooth structure. Additionally, any composition remaining on the tooth structure is preferably substantially the hardened dental composition of the present invention, which can optionally be heated to soften the composition, and thereby allow for easier adhesive removal. Therefore, after orthodontic treatment, one embodiment of FIG. 5 would be where composition layer 22 and additional layer 20 are both hardened orthodontic adhesives. In another embodiment, composition layer 22 would be a hardened orthodontic primer and additional layer 20 would be a hardened orthodontic adhesive.

For another example, FIG. 6 illustrates an embodiment in which orthodontic appliance 10 has two additional dental composition layers (20 and 24) in contact with composition layer 22. Additional layer 20 is between base 12 and composition layer 22. Additional layer 20 is typically an unhardened or hardened dental composition (e.g., an orthodontic adhesive, an orthodontic primer, or a combination thereof). Composition layer 22 can be unhardened or hardened. Additional layer 24 is on composition layer 22 opposite base 12. Additional layer 24 is typically an unhardened dental composition (e.g., an orthodontic adhesive, an orthodontic primer, or a combination thereof). Upon application of orthodontic appliance 10 to a tooth structure, additional layer 24 (and composition layer 22 and additional layer 20, if not already hardened) can be hardened by a variety of methods as described herein above to adhere the orthodontic appliance to the tooth structure.

In some embodiments, additional layer 24 can be a hardenable orthodontic primer that is coated on the tooth structure (and optionally hardened) before the orthodontic appliance with additional layer 20 and composition layer 22 thereon is adhered to the tooth surface.

Upon completion of the orthodontic treatment, at least a portion of hardened composition layer 22 can be heated to reduce the bond strength, and preferably allow fracture within heated composition layer 22 upon removal of the orthodontic appliance. Fracture within heated composition layer 22 results in fracture between, yet safely away from, the orthodontic appliance and the tooth structure. Therefore, after orthodontic treatment, one embodiment of FIG. 6 would be where composition layer 22 and additional layers 20 and 24 are all hardened orthodontic adhesives. In another embodiment, composition layer 22 would be a hardened orthodontic adhesive and additional layers 20 and 24 would be hardened orthodontic primers.

It is to be understood that additional embodiments are contemplated in which additional layers or arrangements of layers are present. Further, the thickness of each layer can be individually varied as desired. Further, dental compositions of the present invention need not be present only in explicitly defined layers, but can also be present distributed uniformly or non-uniformly throughout all or a portion of the layer(s) present on the base of the orthodontic appliance. In some cases a high concentration of a thermally responsive additive may be applied as a particulate layer directly to a hardenable dental composition containing the thermally responsive additive or to a conventional dental composition not including a thermally responsive additive. Further, a thin layer (e.g., a primer) may optionally be applied to the particulate surface to enhance wetting of the tooth upon application of the appliance.

Objects and advantages of this invention are further illustrated by the following examples, but the particular materials and amounts thereof recited in these examples, as well as other conditions and details, should not be construed to unduly limit this invention. Unless otherwise indicated, all parts and percentages are on a weight basis, all water is deionized water, and all molecular weights are weight average molecular weight.

### EXAMPLES

### Test Methods

Shear Bond Strength on Glass Test Method A

Bond strengths of orthodontic brackets adhered to glass with adhesive test samples were measured with an Instron R5500 universal testing machine fitted with an Instron Model 2511-111 500 N load cell (Instron Corp., Canton, Mass.). The crosshead speed was 0.5 cm/min and 10 data points were collected each second. Window glass coupons (15 cm×2.54 cm×0.30 cm) were cleaned with methanol and allowed to dry at room temperature for 5 minutes. Orthodontic brackets (TRANSCEND 6000 or VICTORY Series, 3M Unitek) were adhered to the window glass coupons using an adhesive as described in the subsequent Examples. Curing was done (as described in the subsequent Examples) either thermally in an oven (Model LFD-1-42-3, Despatch, Minneapolis, Minn.); or on a programmable hot plate (Model 400, VWR Scientific, West Chester, Pa.); or photochemically with a Super Spot Max fiber optic 100 W Hg—Xe light source (Lesco, Torrance, Calif.) fitted with a 300-nm long pass optical filter; or with a 3M halogen lamp (Model 5560, 3M Company). The glass coupons with attached brackets were fastened to the Instron crosshead in a clamped mold. An orthodontic standard round wire (0.51 mm, Part number 211-200, 3M Unitek, Monrovia, Calif.) that was clamped to the load cell was passed through the bottom of the bracket such that the brackets were debonded in shear once the crosshead was moved. The maximum stress required to debond the bracket was reported in force per unit area (MPa). Each experiment was repeated typically 3-10 times.

Shear Bond Strength on Glass Test Method B

Bond strengths of orthodontic brackets adhered to glass with adhesive test samples were measured with an Instron Model R5500 universal testing machine fitted with an Instron Model 2511-111 500 N load cell (Instron Corp., Canton, Mass.). The crosshead speed was 0.5 cm/min and 10 data points were collected each second. Window glass coupons (15 cm×2.54 cm×0.30 cm) were cleaned with methanol and allowed to dry at room temperature for 5 minutes. Orthodontic brackets (TRANSCEND 6000, 3M Unitek) were adhered to the window glass coupons using an adhesive as described in the subsequent Examples. Curing was done photochemically with a Super Spot Max fiber optic 100 W Hg—Xe light source (Lesco, Torrance, Calif.) fitted with a 300-nm long pass optical filter. The glass slides with attached brackets were fastened to the Instron crosshead in a clamped mold. An orthodontic standard round wire (0.51 mm, Part number 211-200, 3M Unitek, Monrovia, Calif.) that was clamped to the load cell was passed through the bottom of the bracket such that the brackets were debonded in shear once the crosshead was moved. Prior to debonding (i.e., shearing off the glass surface), the cured adhesives were irradiated for 20 seconds through the brackets with a 75 W QTH blue light gun (Litema Astral Dental, Baden-Baden, Germany) with its dichromic mirror removed. The light gun was gently placed against the bracket surface during the irradiation and the irradiation was continued after the load frame was in motion until debonding occurred. The maximum stress required to debond the bracket was reported in force per unit area (MPa). Each experiment was repeated typically 3-10 times.

Shear Bond Strength on Teeth Test Method A

Bond strengths of orthodontic brackets adhered to teeth with adhesive test samples were determined by the following method. Approximately 10 mg of the adhesive test sample was applied by syringe to the bonding base of a TRANSCEND 6000 (3M Unitek), VICTORY Series (3M Unitek), or CLARITY (3M Unitek) orthodontic bracket. One bracket type would be glass/grit-coated CLARITY Bracket (REF 6400-601 or equivalent; 3M Unitek). The bovine teeth were potted in a fast curing poly(methyl methacrylate) base. The teeth were then cleaned with a pumice aqueous slurry and rinsed. The slightly moist teeth were primed/etched with TRANSBOND Plus Self Etching Primer, REF 712-090 [or with TRANSBOND Plus “SEP” filled with 40% by weight TONE P747 (Example 11)—see Evaluations of Bond Strengths for Examples 1-9 and 12-15], and then dried with moisture- and oil-free air. The ceramic bracket with adhesive was then seated onto the tooth and pressed firmly to extrude any excess adhesive. The excess adhesive was cleaned away and then the adhesive-coated bracket was bonded to the tooth via curing with an ORTHOLUX LED Curing Light (3M Unitek) for 3 sec though the top of the bracket. The samples of teeth and bonded brackets were stored overnight in water at 37° C. and, if heating was desired, transferred to a water bath at 75° C. for either 2 minutes or for 2 hours. Bond strength testing was performed as follows with the heated samples tested as quickly as possible to avoid heat loss. A 0.50-mm round stainless steel wire loop (e.g., Part number 211-200, 3M Unitek) was engaged under the occlusal tie wings of the bracket. Using a Qtest/5 Tester (MTS Systems), a load was applied in a shear/peel mode until debonding from the tooth occurred. The wire attached to the tester was pulled at a rate of 5 mm/minute. The maximum force (in units of pounds) was recorded as bond strength per bracket and the reported value was an average of 10 measurements using 10 different adhesive coated brackets bonded to teeth. This average was then converted to units of MPa by dividing by the bonding base area (square inches) of the bracket and then multiplying by 0.006895.

Shear Bond Strength on Teeth Test Method B

Bond strengths of ceramic orthodontic brackets adhered to bovine teeth with adhesive test samples were measured with a Qtest/5 Tester (MTS Systems). A load was applied in a shear/peel mode until debonding from the tooth occurred. The crosshead speed was 0.5 cm/min. CLARITY (for example REF 6400-601 or equivalent; 3M Unitek) brackets were silane treated by immersion in a 0.5% silane solution using SCOTCHPRIME ceramic primer (3M Company) that was diluted with ethanol (standard SCOTCHPRIME contains 1% silane) followed by air-drying followed by standing for 1 hour in a 600° C. oven. After cooling to room temperature, the brackets were again immersed in the same diluted version of SCOTCHPRIME ceramic primer (0.5% silane) followed by air-drying followed by standing in a 100° C. oven for 1 hour. The brackets were allowed to cool to room temperature. Approximately 10 mg of the adhesive test sample was applied by syringe to the bonding base of the silane-treated CLARITY bracket. The bovine teeth were potted in a fast curing poly(methyl methacrylate) base. The teeth were then cleaned with a pumice aqueous slurry and rinsed.

TRANSBOND PLUS Self Etching Primer (3M Unitek) was applied to the slightly moist bovine teeth and dried with an air jet. Thereafter, the adhesive pre-coated CLARITY brackets were placed on the teeth, excess flash removed and the brackets were cured for 5 seconds with an ORTHOLUX LED Curing light (3M Unitek) to adhere the brackets to the teeth. The specimens were stored at 37° C. for 24 hours and then sheared on the Instron using a Qtest/5 Tester (MTS Systems) as described above. Immediately prior to shear loading the test specimens, they were irradiated with an ORTHOLUX XT light source (3M Unitek) with its IR reflective mirror removed so that it emitted NIR radiation as well as white light. The irradiation was conducted for 60 seconds followed by activation of the load frame on the Qtest/5 Tester with continued irradiation until debonding occurred. The maximum force (in units of pounds) was recorded as bond strength per bracket and the reported value was an average of 5 measurements. This average was then converted to units of MPa by dividing by the bonding base area (square inches) of the bracket and then multiplying by 0.006895.

Shear Bond Strength on Teeth Test Method C

Bond strengths of ceramic orthodontic brackets adhered to bovine teeth with adhesive test samples were measured with a Qtest/5 Tester (MTS Systems). A load was applied in a shear/peel mode until debonding from the tooth occurred. CLARITY (for example REF 6400-601 or equivalent; 3M Unitek) or similar ceramic brackets were optionally silane treated by immersion in a 0.5% silane solution using SCOTCHPRIME ceramic primer (3M Company) that was diluted with ethanol (standard SCOTCHPRIME contains 1% silane) followed by air-drying followed by standing for 1 hour in a 600° C. oven. After cooling to room temperature, the brackets were again immersed in in the same diluted version of SCOTCHPRIME ceramic primer (0.5% silane) followed by air-drying followed by standing in a 100° C. oven for 1 hour. The brackets were allowed to cool to room temperature. Approximately 10 mg of an orthodontic adhesive (3M Company) was applied by syringe to the bonding base of the optionally silane-treated CLARITY bracket. Optionally, the adhesive-coated bracket was then dipped in a wetting agent and pressed into PCL powder as explained in Examples 28A-B and 29A-B. The bovine teeth were potted in a fast curing poly(methyl methacrylate) base. The teeth were then cleaned with a pumice aqueous slurry and rinsed.

TRANSBOND PLUS Self Etching Primer (3M Unitek) was applied to the slightly moist bovine teeth washed with water and dried with an air jet. Thereafter, the CLARITY brackets (pre-treated as described above) were placed on the teeth, excess flash removed and the brackets were cured for 5 seconds with an ORTHOLUX LED Curing Light (3M Unitek) to adhere the brackets to the teeth. The specimens were stored at 37° C. for 24 hours, heated in a water bath at 75° C. for 1 minute, and then immediately shear debonded using a Qtest/5 Tester (MTS Systems) as described in Shear Bond Strength on Teeth Method B above. The maximum force (in units of pounds) was recorded as bond strength per bracket and the reported value was an average of 5 measurements. This average was then converted to units of MPa by dividing by the bonding base area (square inches) of the bracket and then multiplying by 0.006895.

Melt Temperature (Tₘ) Test Method

The melt temperature (Tₘ) of a test sample was determined using a Model 2920 PDSC (Photo Differential Scanning Calorimeter; TA Instruments, New Castle, Del.) if only the heating cycle was recorded; or using a TA Instruments Model 2920 Modulated DSC equipped with a TA Instruments DSC Cooling System if the heating and cooling cycles were recorded. Approximately 8 to 15 mg of a test sample (generally containing 2.5 to 5 wt.-% TONE P767) was loaded into an aluminum pan (Model X1056, Rheometric Scientific, Piscataway, N.J.) and photocured in the PDSC instrument fitted with a 420-nm long pass filter (GG420 Esco Products). The pans were subsequently hermetically sealed with Model X1063 aluminum lids (Rheometric Scientific). The sealed pans were heated at 2° C./min to 100° C. while recording the heat flow. If a cooling cycle was run, it too was run at 2° C./min. The Tₘ values reported represent the endotherm peak maximum and the endotherm energy that were obtained by integration.

### Abbreviations, Descriptions, and Sources of Materials

| Abbreviation | Description and Source of Material |
|---|---|
| HEMA | 2-Hydroxyethyl methacrylate (Sigma-Aldrich, St. Louis, MO) |
| BisGMA | 2,2-Bis[4-(2-hydroxy-3-methacryloyloxypropoxy)phenyl]propane |
| | CAS No. 1565-94-2 |
| Diacryl 101 | BisEMA, Bis(2-hydroxyethyl)bisphenol-A-dimethacrylate (Akzo |
| | Chemicals, Inc., Chicago, IL) |
| TEGDMA | Triethyleneglycol dimethacrylate (Sartomer, Exton, PA) |
| BisEMA6 | Ethoxylated bisphenol A dimethacrylate (Sartomer) |
| PEGDMA- | Polyethyleneglycol dimethacrylate (Sartomer 603; MW about 570; |
| 400 | Sartomer) |
| CDMA | Citric acid dimethacrylate (See Preparation Method described |
| | herein) |
| GDMA-P | Glycerol dimethacrylate phosphate . . . Prepared as described in J. |
| | Dent. Res., 35, 8466 (1956) . . . cited in EP 0 237 233 (Oxman); |
| | (Also, see Example 3 in International Publication WO 02/092021 |
| | (Hecht et al.)) |
| PM-2 | KAYAMER PM-2; Bis(methacryloxyethyl) phosphate |
| | (Nippon Kayaku, Japan) |
| MHP | Methacryloyloxyhexyl phosphate (Prepared as described in |
| | International Publication WO 2005/018581 (Aasen et al.)) |
| IRGACURE | Phosphine oxide photoinitiator (Ciba Specialty Chemicals Corp., |
| 819 | Terrytown, NY) |
| CPQ | Camphorquinone (Sigma-Aldrich) |
| EDMAB | Ethyl 4-(N,N-dimethylamino)benzoate (Sigma-Aldrich) |
| DPIHFP | Diphenyl Iodonium Hexafluorophosphate (Johnson Matthey, |
| | Alpha Aesar Division, Ward Hill, NJ) |
| BHT | 2,6-di-tert-butyl-4-methylphenol (Sigma-Aldrich) |
| EYB | Erythrosin Yellowish Blend Dye (Blend of 90/10 by weight |
| | Erythrosin dye to Eosin Y dye) (Sigma-Aldrich) |
| DBTDL | Dibutyltin dilaurate (Sigma-Aldrich) |
| TPS | Triphenylantimony (Sigma-Aldrich) |
| TRB SH 7080 | Blue-colored sol containing indium-tin-oxide (ITO) nanoparticles |
| | (40 wt.-%) in methyl cellosolve (40 wt.-%) and a urethane acrylate |
| | (20 wt.-%). An efficient NIR-absorber. (Advanced NanoProducts, |
| | S. Korea) |
| NANOTEK | Yellow-green indium-tin-oxide (ITO) powder. An efficient NIR- |
| 0600 ITO | absorber. (Nanophase, Romeoville, IL) |
| EPOLIGHT | Soluble dye known as an efficient NIR-absorber. (Epolin, Inc., |
| 2057 | Newark, NJ) |
| EFKA 4400 | Acrylic polymer dispersant (EFKA, Heerenveen, Netherlands) |
| Filler A | “Control Glass” as described in Example 1 of U.S. Pat. No. |
| | 5,154,762 (Mitra et al.) and subsequently silane-treated as |
| | described for Filler FAS I in U.S. Pat. Publication No. |
| | 2003/0166740 (Mitra et al.). Average particle size estimated to be |
| | 3.0 micrometers. |
| Filler B | Fluoroaluminosilicate Schott glass filler (particle size about 1 |
| | micrometer); silane-treated with A174 silane (OSI Specialties, |
| | West Charleston, WV) |
| Filler C | CAB-O-SIL TS-720 (Cabot Corp.) - Fumed (pyrogenic) silica |
| | surface treated with dimethylsilicone |
| Filler D | Silane-treated, non-aggregated, nano-sized silica filler in the form |
| | of a dry powder was prepared according to the procedure for Filler |
| | A in U.S. Pat. Publication No. 2003/0181541 (Wu et al.), except |
| | that Nalco 2327 was used in place of Nalco 2329 and GENIOSIL |
| | GF-31 (Wacker-Chemie, Munich, Germany) in DOWANOL PM |
| | solvent (Dow Chemical Co., Midland, MI) was used in place of |
| | A174 in methoxy-2-propanol. The nominal particle size of Filler C |
| | was assumed to be the same as in the starting Nalco 2327 silica |
| | sol, i.e., about 20 nanometers. |
| Filler E | Blend of Filler A (49.2 wt.-%), Filler B (49.2 wt.-%), and Filler C, |
| | (1.6 wt.-%) |
| Filler F | Silane-treated zirconia-silica (Zr—Si) filler prepared as |
| | described in U.S. Pat. No. 4,503,169 (Randklev) |
| TONE P767 | Poly(caprolactone) (PCL; Union Carbide, Charleston, WV) |
| | In certain cases, as indicated in specific Examples, the TONE P767 |
| | was finely ground with mortar and pestle prior to use. |
| QPAC-40 | Poly(propylene carbonate) (PPC; Empower Materials Inc., |
| | Newark, DE) |
| DBU | Diazabicyclo[5.4.0]undec-7-ene (AMICURE DBU; Air Products; |
| | Wayne, PA |
| APC PLUS | Orthodontic Adhesive (3M Unitek, Monrovia, CA) |
| APC II | Orthodontic Adhesive (3M Unitek) |
| TB XT | TRANSBOND XT Orthodontic Adhesive (3M Unitek) |
| SCOTCH- | Silane-containing solution (1.0%) also available as 3M ESPE |
| PRIME | RELY-X Ceramic Primer (3M Company) |
| GF-31 | 3-Methacryloyloxypropyltrimethoxysilane (CAS: 2530-85-0) |
| | (Wacker Chemie GmbH, Munich, Germany) |
| BHA | Behenyl acrylate (Cognis, Cincinnati, OH) |
| VAZO 67 | Thermal initiator (DuPont, Wilmington, DE) |
| IEM | 2-Isocyanatoethyl methacrylate (Sigma-Aldrich) or (Kowa |
| | American Corp., New York, NY) |
| HDDA | 1,6-Hexanediol diacrylate (Sigma-Aldrich) |
| Ludox-TM 50 | Colloidal silica; 50% solids in water (Sigma-Aldrich) |
| ODA | Octyldecyl acrylate (Sigma-Aldrich) |
| CERIDUST | Oxidized polyethylene wax particles; average particle diameter = about |
| 3719 | 13 micrometers (Clariant Corp., Charlotte, NC) |
| CERIDUST | Micronized, polar, high-density polyethylene wax particles; |
| 3731 | average particle diameter = about 11 micrometers (Clariant Corp.) |
| MIWAX 411 | Carnauba wax (Michelman Inc., Cincinnati, OH) |

### Starting Materials Preparations

Resins A, B, C and D

Resins A, B, C and D were prepared by combining the ingredients as shown in Table 1A. Resins C and D were prepared by combining the ingredients as shown in Table 1A and heating the resulting mixture to 50-60° C. for 10 to 15 minutes.

TABLE 1A
Compositions of Resins A, B, C and D

| | Resin A | Resin B | Resin C | Resin D |
|---|---|---|---|---|
| Ingredient | Transbond | APC Plus | From AA- | From CRML- |
| (Weight %) | XT | (CESSNA) | 1087 | 1094 |
| HEMA | 0 | 0 | 89.6 | 0 |
| BisGMA | 59.53 | 5.61 | 9.5 | 5.6 |
| CDMA/ | 0 | 79.16 | 0 | 0 |
| PEGDMA- | | | | |
| 400 (1:1) | | | | |
| CDMA | 0 | 0 | 0 | 39.6 |
| Diacryl 101 | 38.52 | 0 | 0 | 0 |
| PEGDMA-400 | 0 | 11.22 | 0 | 50.8 |
| IRGACURE 819 | 0 | 0 | 0.9 | 0 |
| BHT | 0.10 | 0.50 | 0 | 0.50 |
| CPQ | 0.25 | 0.50 | 0 | 0.50 |
| EDMAB | 1.00 | 2.25 | 0 | 2.25 |
| DPIHFP | 0.60 | 0.75 | 0 | 0.75 |
| EYB | 0 | 0.03 | 0 | 0.025 |
| TOTAL: | 100 | 100 | 100 | 100 |

Self-Etching Adhesives A and B

Self-etching Adhesives A and B were prepared by combining the ingredients as shown in Table 1B.

TABLE 1B
Composition of Self-Etching Adhesives A and B

| Ingredient | | |
|---|---|---|
| (Weight %) | Adhesive A | Adhesive B |
| BisEMA6 | 16.9 | 19.1 |
| GDMA-P (25 Wt.-% in TEGDMA) | 0.9 | 0.0 |
| PM-2 | 2.7 | 3.8 |
| MHP | 2.7 | 0.40 |
| Filler F | 75.3 | 76.1 |
| BHT | 0.07 | 0.04 |
| CPQ | 0.23 | 0.08 |
| EDMAB | 0.58 | 0.39 |
| DPIHFP | 0.23 | 0.15 |
| IRGACURE 819 | 0.23 | 0.0 |
| TOTAL: | 100 | 100 |

Preparation of CDMA

In a reaction vessel fitted with a mechanical stirrer, condenser, addition funnel, and air inlet tube, 400 g of citric acid was dissolved in 2 liters of dry THF. To the resultant homogeneous solution was added 0.52 g BHT, 0.5 g TPS, and 0.98 g DBTDL. Dry air was added to the reaction mixture through the inlet tube. Then, 161.5 g (1.04 moles) of IEM was added dropwise through the addition funnel so as to maintain a reaction temperature of about 40° C. The reaction was followed by infrared spectroscopy. After all of the IEM had been added and the IR spectrum showed little to no isocyanate group, the THF was removed under vacuum from the reaction mixture. The resultant viscous liquid was dried. Nuclear magnetic resonance spectroscopy showed the presence of added methacrylate groups and the retention of carboxy groups.

Preparation of Polycaprolactone (PCL) Fibers and Polypropylene Carbonate (PPC)—from AA-1087 and CRML-1094

Electrospinning Solution A. Preparation of 20% w/v Solution of TONE P767E (PCL) in Methyl Ethyl Ketone (MEK): To a 237-ml glass jar was added 80 ml of MEK, and the height of the solvent was marked with a felt-tipped pen on the outside of the jar. The MEK was removed from the jar and TONE P767E (PCL; 16 g) was added. MEK was added back to the jar, up to the marked level. A layer of PTFE tape was placed on the threads of the jar, and a metal-lined metal lid was screwed on tightly by hand. The closed jar was placed onto a shaker table (Eberbach, Ann Arbor, Mich.) and heated with two, 250-Watt IR heat lamps (General Electric), each connected to a rheostat (Staco Energy Products, Dayton, Ohio) at setting of 80-85%. After shaking for 2 hours, the PCL had dissolved in the MEK to afford Electrospinning Solution A that was ready for electrospinning into fibers as described below. A thermocouple was used on the outside of the bottle during the heating, with a reading of 80° C. observed.

Electrospinning Solution B. Preparation of 20% w/v Solution of QPAC-40 (PPC) in MEK: To a 473-ml glass jar was added 100 ml of MEK, and the height of the solvent was marked with a felt-tipped pen on the outside of the jar. The MEK was removed from the jar and QPAC-40 (PPC; 20 g) was added. MEK was added back to the jar, up to the marked level. A layer of PTFE tape was placed on the threads of the jar, and a metal-lined metal lid was screwed on tightly by hand. The closed jar was placed onto a shaker table (Eberbach) and heated with two, 250-Watt IR heat lamps (General Electric), each connected to a rheostat (Staco Energy Products) at setting of 80-85%. After shaking for 3 hours, the PPC had dissolved in the MEK to afford Electrospinning Solution B that was ready for electrospinning into fibers as described below. A thermocouple was used on the outside of the bottle during the heating, with a reading of 80° C. observed.

Electrospinning process: An electrospinning apparatus was prepared using a disposable 60-ml syringe (Beckton-Dickinson, Rutherforsd, N.J.) and a blunt, stainless steel syringe tip (EFD, East Providence, R.I.), attached to a syringe pump (Orion Sage M365, Thermo Electron Corp., Beverly, Mass.). An aluminum panel (0.6 mm×76.2 mm×127 mm; Q-Panel Co., Cleveland, Ohio), that had a small hole (1.27 mm diameter) in the center, was slipped over the syringe tip and positioned approximately 20 mm from the syringe tip. A grounded, high-voltage power supply (CZE 1000R, Spellman High Voltage Electronics, Hauppauge, N.Y.) was connected to the syringe tip in front of the aluminum panel.

An aluminum baking sheet (30.5-cm length×20.3-cm width×2.54-cm depth; Pactiv Corp. Lake Forest, Ill.) was used as the ground plane. Glass slides (optionally coated with ITO to give a resistivity of about 600 ohms/square; available from Optera Inc., Holland, Mich.) were attached to the aluminum pan using double-stick tape, and were positioned so that the slides were 20 cm from the syringe tip. After setting the syringe pump to the desired flow rate and setting the power supply to 20 kV, the power supply was activated to deposit the polymer fibers on the glass slide. Each slide was prepared independently, and for the appropriate time as designated in particular experiment.

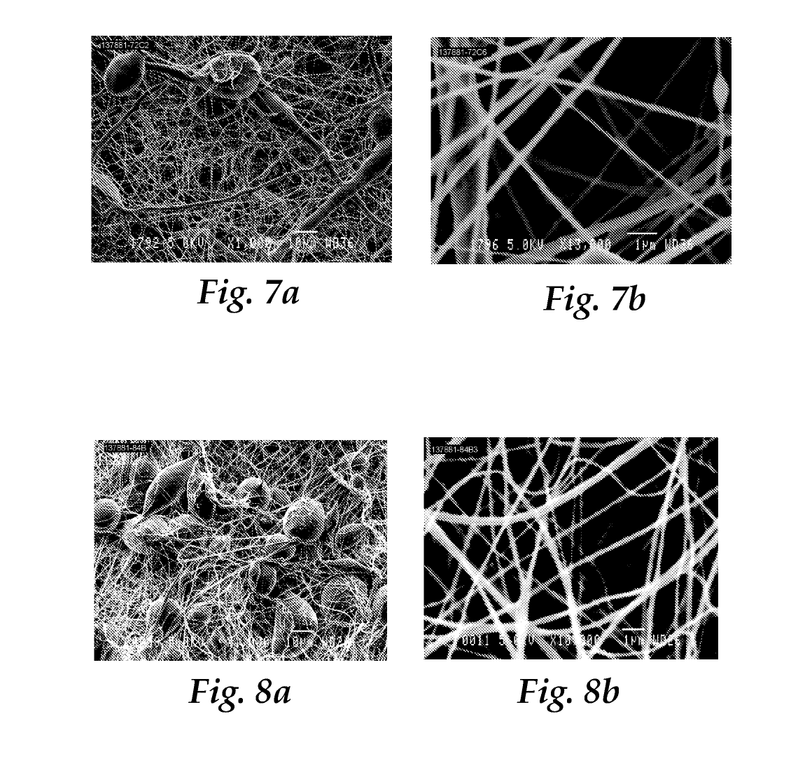

Scanning Electron Microscopy (SEM) of PCL fibers prepared from Electrospinning Solution A using these conditions (10.2 ml/hour flow rate for 90 seconds) showed a random mat of mostly sub-micron fibers with a few fibers having diameters up to 10 microns. Beads are also present on a few of the fibers as shown in FIG. 7.

Preparation of BHA/HEMA-IEM (Behenyl Acrylate/HEMA-IEM Adduct) Polymer

Into a 250-ml round bottom flask with magnetic stirrer was added BHA (18 g), HEMA (2 g), VAZO 67 (0.06 g), toluene (40 g), and ethyl acetate (40 g). This 20% solids solution was degassed using nitrogen for about 10 minutes, sealed, and immersed in a hot oil bath at 65° C. for 20 hours. A 50-g aliquot of the resulting polymeric solution was added to a 450-ml glass jar. Based on the percent solids and the 2-HEMA content in the polymer, the number of moles of hydroxyl groups was calculated and an equivalent number of moles of IEM were added together with a few drops of DBTDL catalyst. The resulting mixture was allowed to agitate for 12 hours at room temperature. The complete consumption of the isocyanate was confirmed by IR spectroscopy.

After solvent removal, the resulting solid was ball milled in a size 000 porcelain jar with 450 g of 10-mm zirconia media (Tosoh YTZ, Tosoh Corporation, Tokyo, Japan) at 100 rpm for 16 hours in 70 g of isopropanol. The media was separated by decanting to yield a colorless suspension. The solvent was removed by rotary evaporation at 25° C. followed by 0.035 torr vacuum for 2 hours and finally by standing under <1 torr vacuum at 30° C. for 18 hours. A fine colorless powder (designated BHA/HEMA-IEM polymer) was obtained and used without further modification. Particle size analysis was done using a CAPA-700 particle size analyzer (Horiba Corporation, Sunnyvale, Calif.). The powder was resuspended in isopropanol by ultrasonication (VibraCell, Sonics and Materials, Inc., Danbury, Conn.) for 5 minutes. The d50 (median) particle size was determined to be 4.1 micrometers by this method.

Preparation of BHA/HDDA (Behenyl Acrylate/Hexanediol Diacrylate) Polymer

Into a beaker equipped with a magnetic stirrer was added deionized water (540 g), Ludox-TM 50 colloidal silica (20.7 g), Promoter A (1.26 g), and HDDA (0.36 g). The resulting mixture was stirred and heated to about 50-65° C. In a separate beaker was heated BHA (360 g) until molten (about 50° C). The molten BHA was slowly added to the aqueous mixture and the pH adjusted to 4-5 with 0.01N HCl. The mixture was then transferred to a reactor vessel and thoroughly mixed with a homogenizer to reduce the monomer droplets average diameter down to less than a micrometer. VAZO 67 initiator (0.54 g) was added, the reactor inserted with nitrogen, and the mixture agitated with a mechanical stirrer (about 300 rpm) and heated to 65° C. for 7-8 hours. After cooling to room temperature, the polymer dispersion was filtered through cheese cloth to remove any coagulum that might be present, and then through a paper filter to isolate the solids. The polymer was washed with water and air dried to afford a white, free-flowing powder (average particle size 10-15 micrometers) that was designated BHA/HDDA Polymer (weight ratio of 1000: 1).

Preparation of BHA/ODA (Behenyl Acrylate/Octyldecyl Acrylate) Polymers

BHA/ODA Polymer was prepared as described above for BHA/HDDA Polymer, except that ODA replaced HDDA in various weight ratios. The BHA/ODA Polymers (weight ratios of 90:10, 70:30, and 50:50) were also isolated as white, free-flowing powders (average particle size 10-15 micrometers).

### Examples 1-4 and Comparative Example 1

### Polycaprolactone (PCL) Dispersed in Adhesive Compositions

Examples 1-4 were prepared by combining Resin A, Filler A, and the thermoplastic additive TONE P767 (PCL) in the amounts shown in Table 2. Comparative Example 1 (CE-1) contained Resin A and Filler A, but no TONE P767. It was observed that compositions having 10% additive or greater became quite thick and grew thicker with time.

TABLE 2
Compositions of Examples 1-4 and Comparative Example 1 (CE-1)

| Ingredient | | | | | |
|---|---|---|---|---|---|
| (Weight %) | CE-1 | Ex. 1 | Ex. 2 | Ex. 3 | Ex. 4 |
| Resin A | 22.75 | 22.8 | 22.8 | 22.8 | 22.8 |
| Filler A | 77.25 | 67.3 | 57.3 | 47.3 | 37.3 |
| TONE P767 | 0 | 10 | 20 | 30 | 40 |
| TOTAL: | 100 | 100 | 100 | 100 | 100 |

### Examples 5-10 and Comparative Example 2

### Adhesive Compositions

Examples 5-9 were prepared by combining Resin B, Filler E, and the thermoplastic additive TONE P767 in the amounts shown in Table 3, followed by mixing in a Speed Mixer (Model DAC-150-FVZ, manufactured by Hauschild & Co, Hamm, Germany and distributed by FlackTek, Inc., Landrum, S.C.) at 3000 rpm for 30 seconds×4 cycles. Comparative Example 2 (CE-2) contained Resin B and Filler E, but no TONE P767.

Example 10 was prepared by combining Resin B, Filler D, and the thermoplastic additive TONE P767 in the amounts shown in Table 3, followed by mixing in a Speed Mixer (Model DAC-150-FVZ). The resulting paste was photocured for 20 seconds using a Model 2500 dental lamp (3M ESPE, St. Paul, Minn.) to afford a hard polymeric material. Thermal data was collected for both uncured and cured samples and the melt temperatures (Tₘ) determined to be 61.96° C. and 64.09° C., respectively, according to the Melt Temperature (Tₘ) Test Method described herein.

TABLE 3
Compositions of Examples 5-10 and Comparative Example 2 (CE-2)

| Ingredient | | | | | | | |
|---|---|---|---|---|---|---|---|
| (Weight %) | CE-2 | Ex. 5 | Ex. 6 | Ex. 7 | Ex. 8 | Ex. 9 | Ex. 10 |
| Resin B | 24 | 24 | 24 | 24 | 24 | 24 | 63.9 |
| Filler E | 76 | 73.5 | 71 | 66 | 61 | 56 | 0 |
| Filler D | 0 | 0 | 0 | 0 | 0 | 0 | 25.9 |
| TONE P767 | 0 | 2.5 | 5 | 10 | 15 | 20 | 10.1 |
| TOTAL: | 100 | 100 | 100 | 100 | 100 | 100 | 100 |

### Example 11

### Primer Composition

Example 11 was prepared by finely grinding the thermoplastic additive TONE P767 with a mortar and pestle and adding the resulting fine powder (40% by weight) into the commercial product TRANSBOND PLUS Self Etching Primer (3M Unitek).

### Examples 12-15 and

### Comparative Example 3 (CE-3) Primer Compositions

Examples 12-15 were prepared by adding the thermoplastic additive TONE P767 into the commercial product SINGLE BOND/TRANSBOND MIP Moisture Insensitive Primer (3M Unitek). The additive was added to the Primer at levels of 0 wt.-% (Comparative Example 3), 5 wt.-% (Example 12), 10 wt.-% (Example 13), 15 wt.-% (Example 14), and 20 wt.-% (Example 15).

### Example 16

### Adhesive Composition

Example 16 was prepared by mixing TONE P767 (30.5%) with Filler D (29.7%) and Resin B (39.8%). Thermal data was collected for a photocured (Model 2500 dental halogen lamp from 3M ESPE for 20 seconds) sample of Example 16 that had been heated, quenched in liquid nitrogen and then reheated. Two melt temperatures (Tₘ) were observed (54.24° C. and 57.70° C.), a result attributed most likely to the formation of two crystalline populations. Reheating and cooling gave rise to entirely reversible behavior.

### Evaluations of Bond Strengths for Examples 1-9 and 12-15

### Debonding of Brackets from Test Surfaces

Examples 12-15 (SINGLE BOND/TRANSBOND MIP Moisture Insensitive Primer containing various levels of TONE P767) were evaluated for shear bond strength using the Shear Bond Strength on Glass Test Method A described herein (with VICTORY Series metal brackets and photocured with a Model 5560 dental lamp). Heating was achieved by placing the samples in a conventional oven.The results were compared to a Comparative Example (CONTROL) of the Primer with no additives. Results at 25° C., 70° C., and 125° C. (each for 24 hours) are provided in Table 4 and showed that debonding was generally facilitated (i.e., lower bond strengths) with increasing temperature and with increasing concentrations of the thermoplastic additive. Samples at the lowest temperature of 25° C. showed little or no bond strength effects with added levels of the additive.

TABLE 4
Debonding of Metal Brackets from Glass Surface.
Shear Bond Strength (MPa) after Exposure at
Various Temperatures

| | TONE P767 | 24 hours at | 24 hours at | 24 hours at |
|---|---|---|---|---|
| Example | (Weight %) | 37° C. | 70° C. | 125° C. |
| CONTROL | 0 | 3173 | 1792 | 1311 |
| (CE-3) | | | | |
| 12 | 5 | 3013 | 1550 | 1365 |
| 13 | 10 | 3527 | 2031 | 213 |
| 14 | 15 | 2713 | 260 | 395 |
| 15 | 20 | 3155 | 1137 | 14 |

Examples 1-4 (Adhesive Compositions containing Resin A and various levels of Filler A and TONE P767) were evaluated for shear bond strength on bovine teeth using the Shear Bond Strength on Teeth Test Method A described herein (with TRANSCEND 6000 ceramic brackets; 3M Unitek). For each Adhesive Composition, the corresponding wt % of Filler A was substituted for Tone P747 such that the total solids in each Composition was the same. The results were compared to Comparative Example 1 (Resin A and Filler A only). Results at 37° C. for 24 hours, and at 37° C. for 24 hours followed by 75° C. for 2 hours are provided in Table 5 and showed that debonding was generally facilitated (i.e., lower bond strengths) with increasing temperature and with increasing concentrations of the thermoplastic additive. Samples at the lowest temperature of 37° C. showed significant loss of bond strength at additive levels of 10% and higher.

TABLE 5
Debonding of Ceramic Brackets from Bovine Teeth Surface.
Shear Bond Strength (MPa) after Exposure at Various
Times and Temperatures

| | TONE P767 | 24 Hours at | 24 Hours at 37° C./2 |
|---|---|---|---|
| Example | (Weight %) | 37° C. | Hours at 75° C. |
| CONTROL (CE-1) | 0 | 11.8 | 9.3 |
| 1 | 10 | 10.4 | 5.4 |
| 2 | 20 | 5.4 | 4.8 |
| 3 | 30 | 5.7 | 3.4 |
| 4 | 40 | 4.4 | 2.6 |

Examples 6-9 (Adhesive Compositions containing Resin B and various levels of Filler E and TONE P767) were evaluated for shear bond strength on bovine teeth using the Shear Bond Strength on Teeth Test Method A described herein (with CLARITY brackets; 3M Unitek; teeth were primed with TRANSBOND PLUS SEP filled with 40% by weight TONE P747). For each Adhesive Composition, the corresponding wt % of Filler E was substituted for TONE P767 such that the total solids in each Composition was the same. The results were compared to Comparative Example 2 (Resin B and Filler E only). Results at 37° C. for 24 hours and at 37° C. for 24 hours followed by 75° C. for 2 hours are provided in Table 6 and showed that debonding was generally facilitated (i.e., lower bond strengths) with increasing temperature and with increasing concentrations of the thermoplastic additive. Samples at the lowest temperature of 37° C. showed significant loss of bond strength at additive levels of 5% and higher.

TABLE 6
Debonding of CLARITY Brackets from Bovine Teeth Surface.
Shear Bond Strength Data (MPa) after Exposure at Various
Times and Temperatures

| | TONE P767 | 24 Hours at | 24 Hours at 37° C./2 |
|---|---|---|---|
| Example | (Weight %) | 37° C. | Hours at 75° C. |
| CONTROL (CE-2) | 0 | 5.8 | 3.7 |
| 6 | 5 | 3.9 | 1.4 |
| 7 | 10 | 3.3 | 1.7 |
| 8 | 15 | 2.5 | 1.1 |
| 9 | 20 | 1.6 | 0.6 |

Examples 5-6 (Adhesive Compositions containing Resin B and various levels of Filler E and TONE P767) were evaluated for Bond Strength on bovine teeth using the Shear Bond Strength on Teeth Test Method A described herein (with CLARITY brackets; 3M Unitek). For each Adhesive Composition, the corresponding wt % of Filler E was substituted for Tone P747 such that the total solids in each Composition was the same. The results were compared to Comparative Example 2 (Resin B and Filler E only). In this evaluation of Examples 5-6, the bovine teeth were pre-treated with Example 11 (TRANSBOND PLUS Self-Etching Primer filled with 40% by weight TONE P747). Results at 37° C. for 24 hours and at 37° C. for 24 hours followed by 75° C. for 2 minutes or followed by 75° C. for 2 hours are provided in Table 7 and showed that debonding was generally facilitated (i.e., lower bond strengths) by the addition of the thermoplastic additive, but that there was only a slight concentration effect between 2.5% and 5.0%. Decreasing the time exposure from 2 hours to 2 minutes had a minimal effect.

TABLE 7
Debonding of CLARITY Brackets from Bovine Teeth Surface.
Shear Bond Strength Data (MPa) after Exposure at
Various Temperatures

| | | | 24 Hours at | 24 Hours at |
|---|---|---|---|---|
| | | | 37° C./ | 37° C./ |
| | TONE P767 | 24 Hours at | 2 Minutes at | 2 Hours at |
| Example | (Weight %) | 37° C. | 75° C. | 75° C. |
| CONTROL | 0 | 4.8 | 2.6 | 2.3 |
| (CE-2) | | | | |
| 5 | 2.5 | 4.9 | 1.7 | 1.3 |
| 6 | 5 | 3.5 | 1.5 | 1.0 |

### Example 17

### Indium Tin Oxide (ITO) Dispersed in HEMA

A dispersion of ITO in HEMA was prepared by combining HEMA (70.0 g), EFKA 4400 Dispersant (0.5 g), and NANOTEK 0600 ITO (10.0 g) and ball-milling for 72 hours at 100 rpm in a size 000 porcelain jar with Tosoh 5-mm YTZ grinding media (450 g). Particle size analysis of the resulting yellow dispersion (Example 17) showed the ITO to have an average particle size of 160 nm.

### Example 18

### Indium Tin Oxide (ITO) Dispersed in Adhesive Resin

A dispersion of ITO in an adhesive resin was prepared by combining Resin B (97 wt.-%) and TRB SH 7080 (3 wt.-%) to provide a dispersion (Example 18) with a 1.2 wt.-% effective concentration of ITO. Upon curing (as a 1-cm diameter disk) with a Model 2500 Dental Blue Lamp (3M Company), the purple-colored monomeric dispersion transformed into a green-colored, transparent polymer.

### Example 19

### ITO Dispersed in Adhesive Resin

A dispersion of ITO in an adhesive resin was prepared by combining Resin B (78.3 wt.-%) and ITO-HEMA (Example 1; 21.7 wt.-%) to provide a dispersion (Example 19) with a 2.7 wt.-% effective concentration of ITO. Upon curing (as a 1-cm diameter disk) with a Model 2500 Dental Blue Lamp (3M Company), the monomeric dispersion transformed into a yellow, opaque polymer.

### Example 20

### Polycaprolactone (PCL) Dispersed in Adhesive Resin

A dispersion of PCL in an adhesive resin (Example 20) was prepared by combining Resin B (95.3 wt.-%) and TONE P767 (4.7 wt.-%) and mixing in a Speed Mixer (DAC-150-FVZ) operating at 3000 rpm for 4×30 seconds.

### Example 21

### PCL and ITO Dispersed in Adhesive Resin

A dispersion of PCL and ITO in an adhesive resin was prepared by combining Resin B (92.1 wt.-%), TONE P767 (5.0 wt.-%) and TRB SH 7080 (2.9 wt.-%) and mixing in a Speed Mixer (DAC-150-FVZ) operating at 3060 rpm for 4×30 seconds to provide a dispersion (Example 21) with a 1.2 wt.-% effective concentration of ITO.

### Example 22

### ITO Dispersed in Adhesive Composite

A dispersion of ITO in a filled adhesive was prepared by combining APC PLUS orthodontic adhesive (97.5 wt.-%) and TRB SH 7080 (2.5 wt.-%) and mixing in a Speed Mixer (DAC-150-FVZ) operating at 3000 rpm for 4×30 seconds to provide a dispersion (Example 22) with a 1.0 wt.-% effective concentration of ITO.

### Example 23

### PCL and ITO Dispersed in Adhesive Composite

A dispersion of PCL and ITO in a filled adhesive was prepared by combining APC PLUS orthodontic adhesive (95.0 wt.-%), TONE P767 (2.5 wt.-%) and TRB SH 7080 (2.5 wt.-%) and mixing in a Speed Mixer (DAC-150-FVZ) operating at 3000 rpm for 4×30 seconds to provide a dispersion (Example 23) with a 1.0 wt.-% effective concentration of ITO.

### Example 24

### Bracket Containing Successive Layers of Adhesive and ITO

A CLARITY bracket (3M Unitek) was silane treated as described in Evaluation D and coated with APC PLUS orthodontic adhesive (3M Unitek). The exposed adhesive end of the coated bracket was then stamped into TRB SH 7080 paste to provide a layer of ITO-containing material. By “stamped” is meant that the bracket was dipped into the low viscosity paste to produce a thin layer of the paste on the outer surface of the adhesive-coated bracket. The coated bracket was stored with the coated layer facing upwards and was evaluated immediately for bond strength.

### Example 25

### Bracket Containing Successive Layers of Adhesive, PCL, and ITO

A CLARITY bracket (3M Unitek) was silane treated as described in Evaluation D and coated with APC PLUS orthodontic adhesive (3M Unitek). The exposed adhesive end of the coated bracket was then sequentially stamped (as generally described in Example 24) into TONE P767 powder and TRB SH 7080 paste to provide sequential layers of PCL-containing material and ITO-containing material. The PCL and ITO layers were designed to be thin compared to the APC PLUS adhesive layer so as to influence fracture away from the bracket-adhesive interface, closer to the tooth for easier clean-up.

### Evaluation A

### Thermographic Analysis of ITO-Containing Cured Adhesive Compositions (Examples 18 and 19)

Cured disk samples of Examples 18 and 19 (ITO dispersions in adhesive resins) and a cured sample of Resin B without added ingredients (CONTROL) were irradiated with a 75 W QTH white light gun (Litema Astral Dental Light gun) with its IR reflecting filter removed. The light gun was held 5.5 mm from the top of the cured disks and a thermal imaging camera (TVS-8502, Avio, Japan) was held about 30 cm from the disks. An image was recorded approximately every 5 seconds, and the apparent temperatures plotted versus time. The measured maximum temperatures of the cured disk samples were both about 250° C., whereas the measured maximum temperature of the Resin B CONTROL was about 50° C. The maximum temperatures of all 3 samples were reached in 10-15 seconds. Therefore, it was demonstrated that a cured dental adhesive containing ITO could be heated rapidly to high temperatures with NIR radiation.

### Evaluation B

### Thermographic Analysis of ITO-Containing Cured Adhesive Compositions (Example 18) on Ceramic Brackets

A sample of Example 18 (ITO dispersion in Resin B) and a sample of Resin B without added ingredients (CONTROL) were used to adhere orthodontic brackets to glass slides according to the following procedure:

Window glass slides (3-mm thick×2.5-cm wide×10-cm long) were cleaned with methanol just prior to use. Approximately 10 mg of the adhesive test sample was placed on the non-fluorescent side of the cleaned slide as a single drop. A TRANSCEND 6000 ceramic bracket (3M Unitek, Monrovia, Calif.) was placed into the drop of adhesive formulation and bonding was carried out by irradiation through the glass slide using a medium pressure Hg spot cure lamp (100 W, Super Spot Max, Lesco, Torrance, Calif.) for 50 seconds.

Immediately after bonding, both brackets were sheared off the glass slides using an Instron R5500 universal testing machine fitted with an Instron Model 2511-111 500-N load cell. The brackets were collected and excess adhesive around the comers of the brackets was chipped away carefully leaving only the hardened adhesive at the base of the brackets. The brackets/hardened adhesives were then heated with NIR radiation as follows.

The brackets were held in an aluminum foil covered white card paper frame with the 75 W QTH Litema Astral Dental Light gun (with its IR reflecting filter removed) held 1 mm away from the top of the bracket on one side of the card paper frame. An Avio thermal imaging camera (TVS-8502 with a close focusing lens) was held approximately 7.5 cm from the base of the bracket at an angle away from the normal to avoid accidental direct radiation from the light gun. The recorded temperature was an apparent temperature assuming that the emissivity of the sample was unity. The temperature rise in the adhesive at the base of the bracket was monitored after the light gun was activated. An image was recorded approximately every 5 seconds. The temperature of the adhesive at the bracket base was plotted versus time and showed that the hardened Example 18 adhesive (with ITO) reached a temperature of about 140° C. after 20 seconds, whereas the CONTROL adhesive (without ITO) reached only about 60° C. after 20 seconds.

These data indicate that it is possible to rapidly heat up a hardened adhesive through the bracket to the Tₘ of a semicrystalline polymer such as PCL in the presence of ITO. This localized heating will cause melting and subsequent debonding at slightly above the Tₘ of the semicrystalline polymer. Furthermore, the use of a QTH source that is not tuned to the absorption spectrum of the ITO suggests that it may be possible to use other energy sources such as NIR LEDs or laser diodes that are tuned to the absorption of the NIR receptor.

### Evaluation C

### Bond Strength Evaluations of PCL- and PCL/ITO-Containing Cured Adhesive Compositions (Examples 20 and 21)

### Debonding of Brackets from Glass Surfaces

Samples of Example 20 (PCL dispersion in Resin B) and Example 21 (PCL and ITO dispersion in Resin B) were used to adhere orthodontic brackets to glass slides as described in Evaluation B. Ten brackets were adhered with each adhesive.

Shear bond strengths were determined using the Shear Bond Strength on Glass Test Method B described herein and included irradiation with the 75 W QTH Litema Astral Dental Light gun (with its dichromic mirror removed). The results are provided in Table 8 and showed that debonding was greatly facilitated (i.e., lower bond strengths) with the presence of the NIR absorber ITO. The estimated temperatures of the samples exposed for 20 seconds to the NIR radiation was <60° C. and >100° C. for Example 20 and Example 21, respectively.

TABLE 8
Debonding of Ceramic Brackets from Glass Surface.
Shear Bond Strength (MPa) after 20-Second Exposure to NIR
Radiation.

| | Adhesive Example 20 | Adhesive Example 21 |
|---|---|---|
| Run | (PCL Additive) | (PCL + ITO Additives) |
| 1 | 5.74 | 3.70 |
| 2 | 4.90 | 1.64 |
| 3 | 4.59 | 2.60 |
| 4 | 4.85 | 2.73 |
| 5 | 4.98 | 2.51 |
| 6 | 4.14 | 1.85 |
| 7 | 3.49 | 2.56 |
| 8 | 4.19 | 1.41 |
| 9 | 4.96 | 1.84 |
| 10 | 5.91 | 2.07 |
| Average: | 4.77 | 2.29 |

The above evaluation was repeated with a new set of cured adhesive samples to determine the effect of aging on adhesion. The adhered-to-glass brackets were maintained at room temperature for 2.5 days under room lights before being debonded as described above. The results are provided in Table 9 and showed again that debonding was greatly facilitated with the presence of the NIR absorber ITO. These data showed more variance than in the above evaluation, but the average values are similar. The lower bond strengths for the ITO-containing samples are due to the much higher temperatures resulting from NIR irradiation through the brackets, in contrast to the samples that did not contain ITO.

TABLE 9
Debonding of Ceramic Brackets from Glass Surface after
Ageing for 2.5 Days at Room Temperature.
Shear Bond Strength (MPa) after 20-Second Exposure to NIR
Radiation.

| | Adhesive Example 20 | Adhesive Example 21 |
|---|---|---|
| Run | (PCL Additive) | (PCL + ITO Additives) |
| 1 | 4.55 | 2.72 |
| 2 | 5.96 | 2.50 |
| 3 | 6.28 | 1.89 |
| 4 | 3.53 | 0.92 |
| 5 | 3.17 | 1.54 |
| 6 | 5.02 | 1.85 |
| 7 | 5.60 | 2.20 |
| 8 | 2.54 | 1.61 |
| 9 | 6.10 | 1.70 |
| 10 | 4.84 | Not Tested |
| Average: | 4.76 | 1.88 |

### Evaluation D

### Bond Strength Evaluations of ITO- and PCL/ITO-Containing Cured Adhesive Compositions (Examples 22 and 23)

### Debonding of CLARITY Brackets from Bovine Teeth Surfaces

Samples of Example 22 (ITO dispersion in APC PLUS Adhesive), Example 23 (PCL and ITO dispersion in APC PLUS Adhesive), and APC PLUS Adhesive without additives (CONTROL) were used to adhere orthodontic brackets to bovine teeth. Five brackets were adhered with each adhesive.

Shear bond strengths using silane-treated CLARITY Brackets adhered to bovine teeth were determined using the Shear Bond Strength on Teeth Test Method B described herein and included NIR irradiation with the ORTHOLUX XT light source (3M Unitek) with its dichromic mirror removed. Additionally, bond strengths of the Example 22, Example 23, and CONTROL samples were run at about room temperature by the same Test Method, except that the NIR irradiation was omitted. The results are provided in Table 10 and showed a trend towards lower bond strengths with the presence of the NIR absorber ITO and a further trend towards lower bond strengths with the added presence of the PCL.

TABLE 10
Debonding of CLARITY Brackets from Bovine Teeth Surfaces.
Shear Bond Strength (MPa) after 60-Second Exposure to NIR
Radiation or No Exposure to NIR Radiation.

| | | |
|---|---|---|
| | | Adhesive |
| | Adhesive | Example 23 |
| CONTROL | Example 22 | (PCL + ITO |
| Adhesive | (ITO Additive) | Additives) |

| Run | No NIR | No NIR | NIR | No NIR | NIR |
|---|---|---|---|---|---|
| 1 | 37.1 | 13.5 | 22.4 | 9.8 | 5.3 |
| 2 | 18.8 | 18.8 | 10.2 | 16.3 | 9.4 |
| 3 | 15.1 | 20.8 | 8.2 | 16.7 | 10.6 |
| 4 | 28.2 | 17.5 | 16.7 | 11.0 | 9.8 |
| 5 | 28.6 | 13.9 | 22.8 | 17.1 | 13.1 |
| Average: | 25.5 | 16.9 | 16.1 | 14.2 | 9.6 |

### Evaluation E

### Bond Strength Evaluations of Cured Adhesive Compositions Containing Layers of ITO or PCL/ITO (Examples 24 and 25)

### Debonding of CLARITY Brackets from Bovine Teeth Surfaces

APC PLUS Adhesive-coated silane-treated CLARITY brackets were prepared as described in Evaluation D (and the Shear Bond Strength on Teeth Test Method B) and then stamped into TRB SH 7080 paste to form a layer of ITO (Example 24) or into TONE P767 powder followed by TRB SH 7080 paste (Example 25) to form layers of PCL and ITO. The reulting layered brackets were allowed to stand for 24 hours at 37° C. and then were debonded as described in Evaluation D (and the Shear Bond Strength on Teeth Test Method B). It is noted that the layering with PCL was found to be difficult, likely due to the relatively large particle size, and as a result several PCL-layered samples failed during the bonding process.

The results are provided in Table 11 and showed that it may be possible to induce selective failure at an interface by layering with ITO and/or PCL materials. The presence of ITO only at the interface would be advantageous from the standpoint of preventing the generation of excess thermal mass.

TABLE 11
Debonding of CLARITY Brackets from Bovine Teeth Surfaces.
Shear Bond Strength (MPa) after 60-Second Exposure to
NIR Radiation or No Exposure to NIR Radiation.

| | |
|---|---|
| Adhesive | Adhesive |
| Example 24 | Example 25 |
| (ITO Layer) | (PCL + ITO Layers) |

| Run | No NIR | NIR | No NIR | NIR |
|---|---|---|---|---|
| 1 | 17.1 | 19.2 | 14.3 | 5.7 |
| 2 | 13.5 | 17.5 | 2.0 | 3.7 |
| 3 | 11.8 | 6.1 | 16.3 | Not Tested |
| 4 | 40.0 | 2.9 | 15.9 | Not Tested |
| 5 | 20.8 | 5.7 | Not Tested | Not Tested |
| Average: | 20.6 | 10.3 | 12.1 | 4.7 |

### Example 26

### PCL Dispersed in Adhesive Composite

A dispersion of PCL in a filled adhesive was prepared by combining APC PLUS orthodontic adhesive (95.0 wt.-%) and TONE P767 (5.0 wt.-%) and mixing in a Speed Mixer (DAC-150-FVZ) operating at 3000 rpm for 4×30 seconds. The resulting adhesive material was designated Example 26.

### Example 27

### PCL Dispersed in Adhesive Composite

A dispersion of PCL in a filled adhesive was prepared by combining TRANSBOND XT orthodontic adhesive (97.5 wt.-%) and TONE P767 (2.5 wt.-%) and mixing in a Speed Mixer (DAC-150-FVZ) operating at 3000 rpm for 4×30 seconds. The resulting adhesive material was designated Example 27.

### Evaluation E

### Bond Strength Evaluations of PCL-Containing Cured Adhesive Compositions (Examples 26 and 27)

### Debonding of CLARITY Brackets from Bovine Teeth Surfaces

Samples of Example 26 (5.0 wt.-% PCL in APC PLUS orthodontic adhesive), Example 27 (2.5 wt.-% PCL in TRANSBOND XT orthodontic adhesive), and the corresponding adhesives without additives (CONTROLS designated as “C”) were applied to silane-treated (0.5% silane) CLARITY brackets and the brackets subsequently adhered to teeth with standard APC PLUS or APC II orthodontic adhesives (3M Unitek). The Example 26 and 27 adhesive materials were applied to the brackets by molding the adhesive to the shape of the bracket base to form a composite-coated base. Shear bond strengths were determined according to the Shear Bond Strength on Teeth Test Method C described herein, except that APC Plus or APC II orthodontic adhesives were used to bond the composite-coated brackets to the teeth surfaces. The assemblies were heated in a water bath at 75° C. for 1 minute or 80° C. for 5 minutes and then debonded as quickly as possible to avoid heat loss.

The results (average of 4-5 replicates) are provided in Table 12 and showed that bond strengths were significantly reduced by using brackets pre-coated with an adhesive composite containing 2.5 to 5.0 wt.-% PCL and heated to 80° C. for 5 minutes during debonding.

TABLE 12
Debonding of Ceramic Brackets from Bovine Teeth Surface.
Shear Bond Strength after Exposure at Various Times and Temperatures

| | | | | | Bond | |
|---|---|---|---|---|---|---|
| | | | | | Strength | |
| | | Orthodontic | | Temperature (Time) | (MPa) | |
| Ex. | Run | Adhesive | Bracket Coating | During Debonding | Mean | S.D. |
| C | 1 | APC PLUS | None | Room Temperature | 11.7 | 1.2 |
| C | 2 | APC PLUS | APC PLUS | Room Temperature | 24.2 | 7.8 |
| 26 | 3 | APC PLUS | APC PLUS + PCL (5%) | Room Temperature | 12.9 | 2.7 |
| 26 | 4 | APC PLUS | APC PLUS + PCL (5%) | 75° C. (1 minute) | 14.5 | 7.3 |
| 26 | 5 | APC PLUS | APC PLUS + PCL (5%) | 80° C. (5 minutes) | 7.8 | 4.1 |
| C | 6 | APC II | None | Room Temperature | 18.7 | 4.3 |
| C | 7 | APC II | TB XT | Room Temperature | 22.1 | 8.4 |
| 27 | 8 | APC II | TB XT + PCL (2.5%) | Room Temperature | 18.7 | 3.3 |
| 27 | 9 | APC II | TB XT + PCL (2.5%) | 75° C. (1 minute) | 11.5 | 3.8 |
| 27 | 10 | APC II | TB XT + PCL (2.5%) | 80° C. (5 minutes) | 9.3 | 4.6 |

### Examples 28A and 28B

### PCL Layered on Adhesive-Coated Bracket

Ceramic brackets were optionally silane-treated, coated with an orthodontic adhesive, covered with a silane wetting agent and dipped in PCL powder to form adhesive-coated brackets having a layer of PCL particles on the outer adhesive surface. The layer of PCL particles provides a potentially weakened (e.g., upon heating) interface between the adhesive and a tooth surface to effect easier bracket debonding (i.e., less force to remove the bracket) and/or easier adhesive clean-up following bracket debonding. The details of preparing such brackets are as follows.

Ten CLARITY ceramic brackets (No. 6400-601; 3M Unitek) were silane treated by immersing the brackets in a cup of 0.5% silane solution prepared by diluting SCOTCHPRIME (3M Company) with ethanol and DI water in a 2.9:1 ratio. The silane treated ceramic brackets were then air dried and then pyrolyzed at 600° C. for 1 hour. After allowing the brackets to cool to room temperature, a second layer of the 0.5% silane solution was coated onto the first, air dried, and then baked at 100° C. for 1 hour. Another ten CLARITY ceramic brackets were not silane treated. All of the twenty brackets were hand-coated with APC PLUS orthodontic adhesive (3M Company) and then 10 brackets (5 silane treated and 5 not silane treated) were dipped into a solution of neat silane (GF3 1) that acted as a “wetting” agent for the subsequent addition of PCL particles. This construction was then lightly stamped onto a mix pad containing TONE P767 (PCL) powder. The resulting bonding surface thus contained an even layer of PCL particles and the excess powder was air blown away for 2-3 seconds. The silane-treated adhesive-coated brackets with a PCL layer were designated Example 28A and the untreated adhesive-coated brackets with a PCL layer were designated Example 28B. The silane-treated and untreated brackets without a PCL layer were designated as CONTROL samples.

### Examples 29A and 29B

### PCL Layered on Adhesive-Coated Bracket

Ceramic brackets were optionally silane-treated, coated with an orthodontic adhesive, covered with TRANSBOND XT primer (TB XT) (3M Unitek) as the wetting agent and dipped in PCL powder to form adhesive-coated brackets having a layer of PCL particles on the outer adhesive surface. The details of preparing such brackets are identical to those described for Examples 28A and 28B, except that the TB XT was substituted for the GF-31 as the wetting agent. The silane-treated adhesive-coated brackets with a PCL layer were designated Example 29A and the untreated adhesive-coated brackets with a PCL layer were designated Example 29B. The silane-treated and untreated brackets without a PCL layer were designated as CONTROL samples.

### Evaluation F

### Bond Strength Evaluations of Cured Adhesive Compositions Having an Outer Layer of PCL Particles (Examples 28A-B and 29A-B)

### Debonding of CLARITY Brackets from Bovine Teeth Surfaces

Othodontic appliance assemblies Example 28A-B and 29A-B and the corresponding assemblies without a PCL layer (CONTROLS designated as “C”) were adhered to bovine teeth and shear bond strengths were determined according to the Shear Bond Strength on Teeth Test Method C described herein, in which the assemblies were optionally heated in a water bath at 75° C. for 1 minute and then debonded as quickly as possible to avoid heat loss.

The results (average of 5 replicates) are provided in Table 13 and showed that bond strengths were reduced by using brackets coated with an orthodontic adhesive containing a PCL particle layer on the outer adhesive surface and heated to 75° C. for 1 minute just prior to debonding.

Table 13 also includes Adhesive Remnant Index (ARI) data. ARI is a term used in the orthodontic industry to visually describe the amount of adhesive remaining on the bonding base of the bracket after debonding. On a scale of 0 to 3, a “3” indicates 0-25% adhesive left on the bracket bonding base or 75-100% of the adhesive being left on the tooth after debonding, meaning failure occurs between the bracket and adhesive. A “2” indicates 25-50% of the adhesive being left on the bracket bonding base or 50-75% of the adhesive being left on the tooth. A “1” indicates 50-75% of the bonding base is covered with adhesive or 25-50% of the adhesive being left on the tooth, and a “0” indicates 75-100% of the bonding base covered with adhesive or 0-25% of the adhesive remaining on the tooth. ARI values close to 0 are indicated for those who prefer easy clean-up. A number in between 0 and 3, for example 1.5, may indicate cohesive failure within the adhesive.

TABLE 13
Debonding of Ceramic Brackets from Bovine Teeth Surface.
Shear Bond Strength after Exposure at Various Temperatures

| | | | | | | Bond | | | |
|---|---|---|---|---|---|---|---|---|---|
| | | Silane | Wetting | | Temp. (Time) | Strength | | | |
| | | Treatment | Agent on | PCL Layer | Just Prior to | (MPa) | | ARI Data | |
| Ex. | Run | on Bracket | Adhesive | on Adhesive | Bonding | Mean | S.D. | Mean | S.D. |
| C | 1 | No | None | None | RT* | 11.6 | 3.5 | 3.0 | 0.0 |
| C | 2 | Yes | None | None | RT | 21.0 | 5.2 | 1.8 | 1.0 |
| 28A | 3 | No | GF-31 | Yes | RT | 7.2 | 0.5 | 1.4 | 1.5 |
| 28B | 4 | Yes | GF-31 | Yes | RT | 14.4 | 8.0 | 0.6 | 1.3 |
| C | 5 | No | None | None | 75° C. (1 min.) | 9.5 | 1.9 | 3.0 | 0.0 |
| C | 6 | Yes | None | None | 75° C. (1 min.) | 17.7 | 3.9 | 1.2 | 1.3 |
| 28A | 7 | No | GF-31 | Yes | 75° C. (1 min.) | 4.2 | 1.8 | 1.2 | 1.6 |
| 28B | 8 | Yes | GF-31 | Yes | 75° C. (1 min.) | 6.3 | 4.4 | 0.0 | 0.0 |
| C | 9 | No | None | None | RT | 12.7 | 2.7 | 3.0 | 0.0 |
| C | 10 | Yes | None | None | RT | 26.9 | 3.3 | 2.0 | 1.4 |
| 29A | 11 | No | TB XT | Yes | RT | 14.7 | 2.5 | 2.6 | 0.9 |
| 29B | 12 | Yes | TB XT | Yes | RT | 21.7 | 4.3 | 0.0 | 0.0 |
| C | 13 | No | None | None | 75° C. (1 min.) | 12.5 | 1.4 | 3.0 | 0.0 |
| C | 14 | Yes | None | None | 75° C. (1 min.) | 24.2 | 3.2 | 2.4 | 1.3 |
| 29A | 15 | No | TB XT | Yes | 75° C. (1 min.) | 8.3 | 2.5 | 2.6 | 0.9 |
| 29B | 16 | Yes | TB XT | Yes | 75° C. (1 min.) | 11.9 | 4.3 | 0.2 | 0.4 |

*RT = Room Temperature

### Examples 30A and 30B

### PCL Dispersed in Self-Etching Adhesive

CLARITY ceramic brackets (REF 6400-601; 3M Unitek) and similar brackets but without a grit or glass layer were silane-treated as described in Examples 28A-B and then coated with approximately 10 mg Self-Etching orthodontic adhesive (Adhesive A) that optionally contained TONE P767 (PCL) to provide the following Control and Example test appliances:

CONTROL A: A CLARITY bracket was coated with Adhesive A.

Example 30A: A CLARITY bracket was coated with Adhesive A (with 2.5 wt.-% PCL).

Example 30B: A ceramic bracket (free of grit or glass) was precoated with TB XT adhesive as follows: An XT syringe was used to apply as thin layer of TB XT adhesive to the bracket base and then the adhesive was pushed against a mixing pad to provide a thin, smooth layer of new adhesive on the base; the flash was cleaned and the adhesive cured with an ORTHOLUX LED for 5 seconds. Adhesive A (with 2.5 wt.-% PCL) was then coated onto the ceramic bracket containing a smooth, cured organic bonding base.

### Example 31

### PCL Dispersed in Self-Etching Adhesive

CLARITY ceramic brackets (REF 6400-601; 3M Unitek) and similar brackets but without a grit or glass layer were optionally silane-treated as described in Examples 28A-B and then coated with approximately 10 mg TB XT orthodontic adhesive or Self-Etching orthodontic adhesive (Adhesive B) that optionally contained TONE P767 (PCL) to provide the following Control and Example test appliance assemblies:

CONTROL B: An untreated CLARITY bracket was coated with TB XT Adhesive.

CONTROL C: An untreated CLARITY bracket was coated with Adhesive B.

CONTROL D: A silane-treated CLARITY bracket was coated with Adhesive B.

CONTROL E: A silane-treated ceramic bracket (free of grit or glass) was coated with Adhesive B.

CONTROL F: A silane-treated ceramic bracket (free of grit or glass) was precoated with TB XT adhesive as follows: An XT syringe was used to apply a thin layer of TB XT adhesive to the bracket base and then the adhesive was pushed against a mixing pad to provide a thin, smooth layer of an organic layer on the ceramic bracket; the flash was cleaned and the adhesive cured with an ORTHOLUX LED for 5 seconds. Adhesive B was then coated onto the ceramic bracket which contained a smooth, cured organic bonding base (in contrast to a glass/gritted bonding base which is standard for CLARITY brackets). The objective was to create an excellent bond between the organic bonding base and the self etching adhesive containing PCL such that at debonding, the adhesive-tooth interface would preferably fail over the adhesive-bonding base interface, providing less adhesive on the tooth for easier clean-up.

Example 31: A silane-treated ceramic bracket (free of grit or glass) was precoated with TB XT adhesive as described in CONTROL F. Adhesive B (with 2.5 wt.-% PCL) was then coated onto the ceramic bracket which contained a smooth, cured organic bonding base.

### Evaluation G

### Bond Strength Evaluations of Cured Self-Etching Adhesive Compositions Having PCL Particles Dispersed in the Compositions (Examples 30A-B and 31)

### Debonding of CLARITY Brackets from Bovine Teeth Surfaces

Orthodontic appliance assemblies Example 30A-B and 31 and the corresponding CONTROLS A-F were adhered to bovine teeth and shear bond strengths were determined according to the Shear Bond Strength on Teeth Test Method C described herein, except that there was no pretreatment of the bovine teeth with TRANSBOND PLUS SEP self-etching primer. The assemblies were optionally heated in a water bath at 75° C. for 1 minute and then debonded as quickly as possible to avoid heat loss.

The results (average of 5 replicates) are provided in Table 14 and showed that bond strengths were reduced by using brackets coated with a self-etching orthodontic adhesive containing dispersed PCL particles and heated to 75° C. for 1 minute just prior to debonding.

TABLE 14
Debonding of Ceramic Brackets from Bovine Teeth Surface.
Shear Bond Strength after Exposure at Various Temperatures

| | | | | Bonding | | | |
|---|---|---|---|---|---|---|---|
| | | | Temp. (Time) | Strength | | | |
| | Orthodontic | | Just Prior to | (MPa) | | ARI Data | |
| Example/Run | Adhesive | Bracket/Bracket Coating | Debonding | Mean | S.D. | Mean | S.D. |
| CONTROL A | Adhesive A | CLARITY/Silane (0.5%) | RT | 6.4 | 1.1 | 0.0 | 0.0 |
| 30A/Run 1 | Adhesive A | CLARITY/Silane (0.5%) | RT | 6.3 | 2.5 | 0.0 | 0.0 |
| | (2.5% PCL) | | | | | | |
| 30A/Run 2 | Adhesive A | CLARITY/Silane (0.5%) | 75° C. (1 min.) | 4.2 | 2.9 | 0.0 | 0.0 |
| | (2.5% PCL) | | | | | | |
| 30B/Run 1 | Adhesive A | Ceramic (free of glass or grit)/ | RT | 3.8 | 2.5 | 0.0 | 0.0 |
| | (2.5% PCL) | Silane (0.5%) | | | | | |
| | | Precoated with cured TB XT | | | | | |
| 30B/Run 2 | Adhesive A | Ceramic (free of glass or grit)/ | 75° C. (1 min.) | 2.7 | 1.7 | 0.0 | 0.0 |
| | (2.5% PCL) | Silane (0.5%) | | | | | |
| | | Precoated with cured TB XT | | | | | |
| CONTROL B | TB XT | CLARITY/Untreated | RT | 12.9 | 2.1 | 3.0 | 0.0 |
| CONTROL C | Adhesive B | CLARITY/Untreated | RT | 6.2 | 2.6 | 0.0 | 0.0 |
| CONTROL D | Adhesive B | CLARITY/Silane (0.5%) | RT | 8.2 | 2.4 | 0.0 | 0.0 |
| CONTROL E | Adhesive B | Ceramic (free of glass or grit)/ | RT | 9.3 | 3.1 | 1.0 | 1.4 |
| | | Silane (0.5%) | | | | | |
| CONTROL F | Adhesive B | Ceramic (free of glass or grit)/ | RT | 9.8 | 5.8 | 0.8 | 1.3 |
| | | Silane (0.5%) | | | | | |
| | | Precoated with cured TB XT | | | | | |
| 31/Run 1 | Adhesive B | Ceramic (free of glass or grit)/ | RT | 7.0 | 2.6 | 1.3 | 1.5 |
| | (2.5% PCL) | Silane (0.5%) | | | | | |
| | | Precoated with cured TB XT | | | | | |
| 31/Run 2 | Adhesive B | Ceramic (free of glass or grit)/ | 75° C. (1 min.) | 5.5 | 2.7 | 0.8 | 1.5 |
| | (2.5% PCL) | Silane (0.5%) | | | | | |
| | | Precoated with cured TB XT | | | | | |

### Example 32 and Comparative Example 4 (CE-4)

### PCL Fibers Layered on Cured Adhesive

One drop (approximately 10 mg) of adhesive (Resin C; HEMA-89.6 wt %, bisGMA-9.5 wt %, and IRGACURE 819-0.9%) was placed on each of two ITO-coated glass slides (resistivity of about 600 ohms/square; available from Optera Inc., Holland, Mich.) with or without a deposit of PCL fibers. (See Starting Material Preparations.) The adhesive remained as a bead on the surface of the slide without PCL fibers, but immediately wet the slide with deposited PCL fibers. In both cases, another drop of adhesive was added to the prior drop, followed by placement of an orthodontic bracket (TRANSCEND 6000, 3M Unitek) to the adhesive drops on each of the slides. There was considerable wetting of the PCL fibers by this procedure.

The brackets were cured and adhered to the slides by irradiation for 30 seconds with a Super Spot Max fiber optic 100 W Hg—Xe light source (Lesco, Torrance, Calif.) and held 39-mm from the brackets. The resulting bracket assemblies were designated Example 32 (with PCL fibers) and Comparative Example 4 (CE-4) (without PCL fibers).

### Example 33 and Comparative Example 5 (CE-5)

### PCL Fibers Layered on Cured Adhesive

Example 33 (with PCL fibers) and Comparative Example 5 (CE-5) (without PCL fibers) were prepared as described for Example 32 and CE-4, except that the initial drop of Resin C was immediately followed by placement of the bracket, such that the adhesive did not have time to spread across the PCL surface and thus another drop of adhesive was not required or used. The brackets were cured and adhered to the slides as described for Example 32.

### Evaluation H

### Bond Strength Evaluations of Cured Adhesive Compositions Layered on PCL Fibers (Examples 32 and 33)

### Debonding of Brackets (TRANSCEND 6000) from ITO-Treated Glass Surfaces

A 1000-g weight was hung from the brackets while simultaneously heating the back of the glass slide with a 250-Watt IR lamp (General Electric) operating at full power. The time to failure as well as the temperature at failure was recorded. The failure mode was also recorded for all brackets. The distance from the lamp to the back of the glass slide was 5 cm. Resulting data including Fiber Spinning Time (reflecting the quantity of PCL fibers on the glass slides), Trans (%) at 550 nm (measured on a Hewlett Packard 8452A Spectrophotometer interfaced with a PC running HP UV-JVis ChemStation software), Time of Failure, Temperature at Failure, and Failure Mode are provided in Table 15.

TABLE 15
Results for Examples 32-33 and Comparative Examples 4-5 (CE-4 and
CE-5)

| | Fiber | Trans | Time of | Temp. | |
|---|---|---|---|---|---|
| Example/ | Spinning | (%) at | Failure | (° C.) at | |
| Run | Time | 550 nm | (min:sec) | Failure | Failure mode |
| CE-4/ | Control | 79.2 | 8:45 | 130 | Adhesive Failure |
| Run 1 | | | | | at glass surface |
| CE-4/ | Control | 79.4 | 10:00 | 121 | Adhesive Failure |
| Run 2 | | | | | at glass surface |
| 32/Run 3 | 5 sec | 39.6 | 5:44 | 119 | Adhesive Failure |
| | | | | | at glass surface |
| 32/Run 4 | 5 sec | 41.9 | 1:43 | 84 | Adhesive Failure |
| | | | | | at glass surface |
| 32/Run 5 | 10 sec | 23.2 | 3:49 | 116 | Adhesive Failure |
| | | | | | at glass surface |
| 32/Run 6 | 10 sec | 22.5 | 2:43 | 112 | Adhesive Failure |
| | | | | | at glass surface |
| 32/Run 7 | 15 sec | 16.1 | 5:27 | 133 | Adhesive Failure |
| | | | | | at glass surface |
| 32/Run 8 | 15 sec | 13.4 | 7:04 | 142 | Adhesive Failure |
| | | | | | at glass surface |
| CE-5/ | Control | 79.7 | 2:18 | 100 | Adhesive Failure |
| | Run 9 | | | | at glass surface |
| CE-5/ | Control | 79.5 | 3:22 | 113 | Adhesive Failure |
| Run 10 | | | | | at glass surface |
| CE-5/ | Control | 80 | 3:15 | 100 | Cohesive failure |
| Run 11 | | | | | 70% left on glass |
| 33/Run 12 | 5 sec | 51.2 | 1:27 | 81 | Adhesive Failure |
| | | | | | at glass surface |
| 33/Run 13 | 5 sec | 45.5 | 1:18 | 96 | Adhesive Failure |
| | | | | | at glass surface |
| 33/Run 14 | 5 sec | 55.5 | 0:45 | 60 | Adhesive Failure |
| | | | | | at glass surface |
| 33/Run 15 | 10 sec | 26.9 | 0:52 | 69 | Adhesive Failure |
| | | | | | at glass surface |
| 33/Run 16 | 10 sec | 23.9 | 1:27 | 81 | Adhesive Failure |
| | | | | | at bracket |
| 33/Run 17 | 10 sec | 22.6 | 1:05 | 75 | Adhesive Failure |
| | | | | | at glass surface |
| 33/Run 18 | 15 sec | 18.5 | 0:44 | 69 | Adhesive Failure |
| | | | | | at glass surface |
| 33/Run 19 | 15 sec | 17.5 | 1:07 | 87 | Adhesive Failure |
| | | | | | at glass surface |
| 33/Run 20 | 15 sec | 20.2 | 1:12 | 91 | Adhesive Failure |
| | | | | | at glass surface |
| 33/Run 21 | 20 sec | 15.4 | 1:33 | 90 | Adhesive Failure |
| | | | | | at glass surface |
| 33/Run 22 | 20 sec | 16.4 | 1:11 | 91 | Adhesive Failure |
| | | | | | at glass surface |
| 33/Run 23 | 20 sec | 17.1 | 1:23 | 81 | Adhesive Failure |
| | | | | | at glass surface |

Visible melting of the PCL was evident on heating the brackets. The PCL appeared to turn clear from hazy white on melting. Bracket failure was observed to be sudden with no visual evidence of creep. It should be pointed out that the temperatures at failure indicated in Table 15 were measured at a short distance above the bracket (closer to the lamp) and may not be indicative of the temperatures at the PCL-bracket interface.

While the results for Example 32 show loss in adhesion as expected in the presence of PCL, there is no clear pattern that shows progressive reduction of time to failure upon increasing the PCL fiber concentration on the glass slide. There is also significant variance in the data.

The results for Example 33 (that avoids the use of excess adhesive) show less variance and demonstrate a consistent pattern of adhesion loss upon melting of the PCL layer. The measured temperature at failure appears to be reasonable given that the melting point of PCL (TONE P767) is in the 57° C. to 64° C. region depending on the thermal history of the material.

The Example 33 data are consistent with the concept of debonding by the placement of fibers (preferably nanofibers) of thermoplastic materials at interfaces. It appears from Table 15 that even a very sparse layer of PCL fibers (e.g., see Example 33/Runs 12-13; “5 seconds” Fiber Spinning Time) is sufficient to affect debonding times. These results suggest that even slight modifications in the bonding interface can bring about reliable and efficient debonding. The use of thermoplastics (e.g., PCL fibers) at the tooth-adhesive interface should allow on demand debonding with the application of heat.

### Example 34A-B and Comparative Example 6

### PCL Fibers Containing ITO Nanoparticles and Layered on Cured Adhesive

In a glass vial was placed a sample (10.0 g) of Electrospinning Solution A (20% TONE P767 in MEK) and TRB SH 7080 (0.30 g). The mixture was hand stirred with a wooden stick to give a cloudy, blue solution that was transferred to a 60-ml disposable syringe to perform the electrospinning. The electrospinning was performed as described in Starting Material Preparations with the flow rate set at 10.2 ml/hour. Transmission measurements were used to monitor the density of fibers on the glass slides to help obtain similar densities to the ones that did not contain the ITO particles. Scanning electron microscopy of fibers prepared using these conditions show a random mat of mostly sub-micron fibers with a few fibers having diameters up to 10 microns. Large beads are also present on a few of the fibers as shown in the micrographs in FIGS. 8a-b. The PCL plus ITO fiber mats were designated Example 34A. Corresponding PCL (without ITO) fiber mats were also prepared.

One drop (approximately 10 mg) of adhesive (Resin D; See Starting Material Preparations) was placed on each of two glass slides with either a PCL/ITO fiber mat (Example 34A) or a PCL only fiber mat. Immediately, an orthodontic bracket (TRANSCEND 6000, 3M Unitek) was placed onto each of the adhesive drops on each of the glass slides. The brackets were immediately cured and adhered to the slides by irradiation for 20 seconds with a Curing Light 2500 (3M ESPE), positioned such that the optical fiber tip rested lightly on the bracket. The resulting bracket assemblies were designated Example 34B (with PCL/ITO fibers) and Comparative Example 6 (CE-6) (with PCL only fibers).

### Example 35A-B and Comparative Example 7

### PPC Fibers Containing ITO Nanoparticles and Layered on Cured Adhesive

In a glass vial was placed a sample (10 g) of Electrospinning Solution B (20%; 10 g QPAC-40 in MEK), TRB SH 7080 (0.60 g), and DBU (0.09 g). (Polycarbonates have been shown to have base sensitivity; therefore, to effect a lower degradation temperature, diazabicyclo[5.4.0]undec-7-ene (DBU) was added to the spinning formulation at the 1% level. For the formulations containing the ITO nanoparticles, the QPAC-40 solution and the TRB SH 7080 were combined an a glass vial and mixed by hand stirring before the DBU was added and stirred in by hand.)

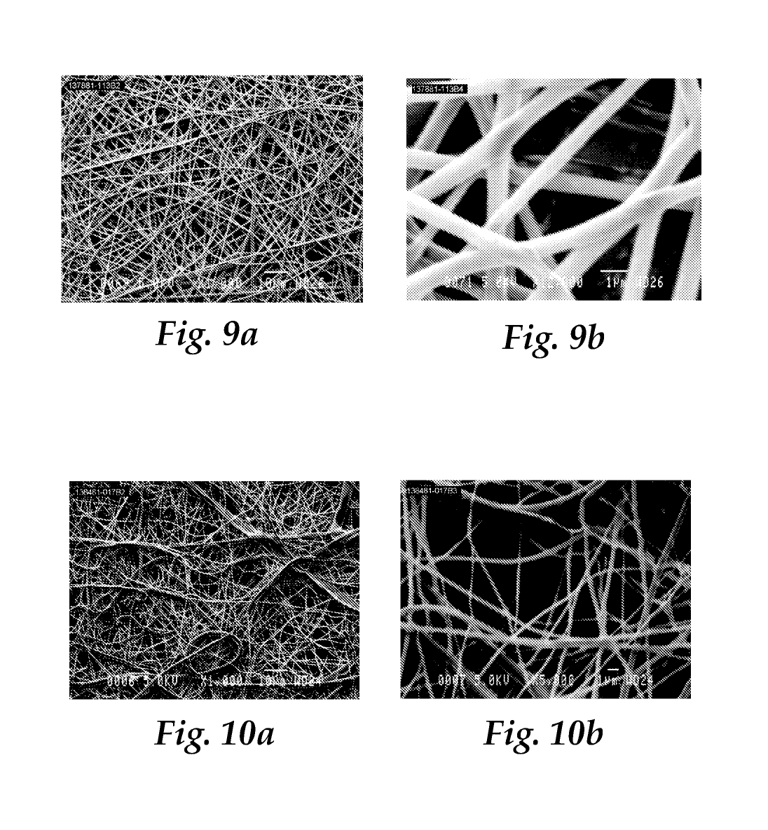

The electrospinning was performed as described above (See Starting Material Preparations) with the flow rate set at 2.0 ml/hour and a time duration of approximately 120 seconds. Transmission measurements were used to monitor the density of fibers on the glass slides and many required additional electrospinning to give transmittances close to what was seen on the control slides (without ITO particles). During the electrospinning process, liquid droplets were depositing on the aluminum ground plane and the glass slides; therefore, a new spinning formulation was prepared that contained Electrospinning Solution B (5 g, 20% QPAC-40 in MEK), TRB SH 7080 (0.30 g), and DBU (0.05 g). The last two slides (Example 36/Runs 23-24) were coated with this new spinning formulation. Scanning electron microscopy (FIGS. 9a-b) of fibers prepared using these conditions showed a mat of fibers that have a very uniform fiber diameter distribution between 600 nm and 1000 nm. The PPC/DBU plus ITO fiber mats were designated Example 35A. Corresponding PPC/DBU (without ITO) fiber mats were also prepared.

One drop (approximately 10 mg) of adhesive (Resin D; See Starting Material Preparations) was placed on each of glass slides with either a PPC/DBU/ITO fiber mat (Example 35A) or a PPC/DBU only fiber mat. Immediately, an orthodontic bracket (TRANSCEND 6000, 3M Unitek) was placed onto each of the adhesive drops on each of the glass slides. The brackets were immediately cured and adhered to the slides by irradiation for 20 seconds with a Curing Light 2500 (3M ESPE), positioned such that the optical fiber tip rested lightly on the bracket. The resulting bracket assemblies were designated Example 35B (with PPC/DBU/ITO fibers) and Comparative Example 7 (CE-7) (with PCL/DBU only fibers).

### Example 36A-B and Comparative Example 8

### PCL Fibers Containing NIR (Near IR) Absorbing Dye and Layered on Cured Adhesive

In a glass vial was placed a sample (10.0 g) of Electrospinning Solution A (20% TONE P767 in MEK) and EPOLIGHT 2057 dye (0.033 g). The mixture was hand stirred with a wooden stick to give a green solution that was transferred to a 60-ml disposable syringe to perform the electrospinning. The electrospinning was performed as described in Starting Material Preparations with the flow rate set at 2.0 ml/hour. Transmission measurements were used to monitor the density of fibers on the glass slides to help obtain similar densities to the ones that did not contain the dye. Scanning electron microscopy of fibers prepared using these conditions show a random mat of mostly sub-micron fibers with a few fibers (<5%) having diameters up to 10 microns. Large beads are also present on a few of the fibers as shown in the micrographs in FIGS. 10a-b. The PCL plus dye fiber mats were designated Example 36A. Corresponding PCL (without dye) fiber mats were also prepared.

Adhesive (Resin D) was used to adhere brackets to glass slides as described for Example 34B. The resulting bracket assemblies were designated Example 36B (with PCL/Dye fibers) and Comparative Example 8 (CE-8) (with PCL only fibers).

### Evaluation I

### Bond Strength Evaluations of Cured Adhesive Compositions Layered on PCL/ITO, PPC/DBU/ITO, and PCL/Dye Fiber Mats (Examples 34B, 35B and 36B)

### Debonding of Brackets (TRANSCEND 6000) from Glass Surfaces

A 1500-g weight was hung from the brackets while simultaneously irradiating the back of the glass slide with a 75 W QTH white light gun (Litema Astral Dental Light gun) with its IR reflecting filter removed. The light gun was positioned such that the end of the optical fiber was touching the glass slide, directly behind the adhered bracket. The time to failure as well as the temperature at failure was recorded. The failure mode was also recorded for all brackets. Resulting data including Transmittance (%) at 550 nm (measured on a Hewlett Packard 8452A Spectrophotometer interfaced with a PC running HP UV-Vis ChemStation software), Time of Failure, and Failure Mode are provided in Table 16.

TABLE 16
Results for Examples 34B, 35B, 36B and Comparative Examples 6-8
(CE-6, CE-7 and CE-8)

| | | Trans (%) | Time of Failure | |
|---|---|---|---|---|
| Example | Run | at 550 nm | (min:sec) | Failure Mode |
| CE-6 | 1 | 1.005 | >3:00 | None |
| | 2 | 1.739 | >3:00 | None |
| | 3 | 1.976 | 2:06 | Adhesive Failure at glass |
| | | | | surface |
| | 4 | 1.106 | 2:12 | Adhesive Failure at glass |
| | | | | surface |
| | 5 | 2.481 | >3:00 | None |
| 34B | 6 | 4.02 | 1:10 | Adhesive Failure at glass |
| | | | | surface |
| | 7 | 1.697 | 0:52 | Adhesive Failure at glass |
| | | | | surface |
| | 8 | 3.447 | 1:25 | Adhesive Failure at glass |
| | | | | surface |
| | 9 | 0.679 | 0:55 | Adhesive Failure at glass |
| | | | | surface |
| | 10 | 0.713 | 1:15 | Adhesive Failure at glass |
| | | | | surface |
| CE-7 | 11 | 4.284 | >3:00 | None |
| | 12 | 2.086 | 2:50 | Mixed |
| | 13 | 2.118 | >3:00 | None |
| | 14 | 5.135 | >3:00 | None |
| | 15 | 5.495 | >3:00 | None |
| 35B | 16 | 2.418 | 1:28 | Adhesive Failure at glass |
| | | | | surface |
| | 17 | 4.551 | 0:17 | Mixed |
| | 18 | 5.549 | 0:22 | Adhesive Failure at glass |
| | | | | surface |
| | 19 | 4.596 | 1:33 | Adhesive Failure at glass |
| | | | | surface |
| CE-8 | 20 | 4.634 | >3:00 | None |
| 36B | 21 | 4.251 | 1:36 | Adhesive Failure at glass |
| | | | | surface |
| | 22 | 4.122 | 0:04 | Adhesive Failure at glass |
| | | | | surface |
| | 23 | 3.838 | 0:58 | Adhesive Failure at glass |
| | | | | surface |
| | 24 | 3.889 | 1:39 | Adhesive Failure at glass |
| | | | | surface |

Visible melting of the PCL fibers (Examples 34B and CE-6) was evident after irradiating the brackets for 20 seconds. The PCL appeared to turn clear from hazy white on melting. Bracket failure was observed to be sudden with no visual evidence of creep. The data from Table 16 show that bonded brackets with a fiber mat of only PCL (CE-6) show little or no changes in adhesive strength when irradiated with the 75-Watt QTH bulb; whereas, a significant decrease in adhesive strength was observed when samples having the ITO-containing PCL nanofibers (Example 34B) were irradiated.

Visible melting and degradation of the PPC fibers (Examples 35B and CE-7) were evident afrer irradiating the brackets for 30 seconds. The cloudy fiber mat changed to a clear ring around the bound bracket. Bracket failure was observed to be sudden with no visual evidence of creep. One of the control samples (Run 12) showed a mixed failure mode; meaning, that complete adhesive failure at the adhesive/glass interface was observed directly behind the bracket, but that cohesive failure of the adhesive was observed at the top edge of the bracket with a sharp break of the adhesive at the edge of the bracket. Some adhesive was left on the glass substrate above the bracket, but the adhesive below the bracket was removed. The data from Table 16 show that bonded brackets with a fiber mat of only PPC/DBU (CE-7) show little or no changes in adhesive strength when irradiated with the 75-Watt QTH bulb; whereas, a significant decrease in adhesive strength was observed when samples having the ITO-containing PPC/DBU nanofibers (Example 35B) were irradiated.

Visible melting of the PCL fibers (Examples 36B and CE-8) was evident after irradiating the brackets for 20 seconds. The PCL appeared to turn clear, from hazy white, on melting. Bracket failure was observed to be sudden with no visual evidence of creep. The data from Table 16 show a decrease in adhesive strength (vs. CE-8) when the PCL nanofibers containing the EPOLIGHT dye were irradiated with the 75-Watt QTH bulb.

### Example 37

### BHA/HEMA-IEM Particles and ITO Dispersed in Adhesive Resin

A dispersion of a thermally responsive material and ITO in an adhesive resin was prepared by combining Resin A (71.2 wt.-%), BHA/HEMA-IEM Particles (24.3 wt.-%; see Starting Materials Preparation) and TRB SH 7080 (4.5 wt.-%) and mixing in a Speed Mixer (Model DAC-150-FVZ) at 3000 rpm for 60 seconds×3 cycles to provide a dispersion (Example 37) with a 1.8 wt.-% effective concentration of ITO.

### Evaluation J

### Bond Strength Evaluations of BHA/HEMA-IEM Particles-Containing Cured Adhesive Composition (Examples 37)

### Debonding of TRANSCEND 6000 Brackets from Bovine Teeth Surfaces

Samples of Example 37 were used to adhere orthodontic brackets to bovine teeth. Five brackets were adhered for each of the two procedures utilized.

Shear bond strengths using TRANSCEND 6000 Brackets adhered to bovine teeth were determined using generally the Shear Bond Strength on Teeth Test Method A described herein, except that a dental halogen lamp Model 2500 (3M ESPE) was used for 20 seconds to adhere the brackets to the teeth. For Procedure A, the brackets were shear debonded on the Instron R5500 as described in Test Method A. For Procedure B, the brackets were loaded onto the Instron instrument and irradiated for 10 seconds with a Cuda fiber optic light source (Model I-100, Sunoptic Technologies, Jacksonville, Fla.) that was fitted with a quartz-tungsten-halogen lamp possessing a gold-coated ellipsoidal reflector (Model L6408-G, Gilway Scientific, Woburn, Mass.) and a NIR transmitting optical fiber (Newport Oriel, Stratford, Conn.). The Instron loadframe was activated immediately following the 10-second NIR exposure. The results are provided in Table 17 and show the significantly reduced bond strength of samples following the 10-second NIR exposure as compared to the samples without NIR exposure.

TABLE 17
Debonding of TRANSCEND Brackets from Bovine Teeth Surface.
Shear Bond Strength (MPa)

| | Example 37 | Example 37 |
|---|---|---|
| Run | Procedure A (No NIR) | Procedure A (10-Second NIR) |
| 1 | 8.39 | 3.20 |
| 2 | 9.83 | 4.45 |
| 3 | 9.85 | 3.72 |
| 4 | 6.86 | 3.56 |
| 5 | 6.98 | 2.32 |
| Average: | 8.38 | 3.45 |

### Example 38

### PCL Dispersed in an Orthodontic Adhesive

A dispersion of PCL in an orthodontic adhesive was prepared by combining TONE P767 (2.5 wt.-%) and APC PLUS orthodontic adhesive and mixing in a Speed Mixer (DAC-150-FVZ) operating at 3000 rpm for 4×30 seconds to provide a dispersion (Example 38).

### Example 39

### BHA/HDDA Polymer Dispersed in an Orthodontic Adhesive

A dispersion of a thermally responsive polymer in an orthodontic adhesive was prepared by combining BHA/HDDA Polymer (4.0 wt.-%; 1000:1 weight ratio; see Starting Materials Preparations) and APC PLUS orthodontic adhesive, and mixing in a Speed Mixer (DAC-150-FVZ) operating at 3000 rpm for 4×30 seconds to provide a dispersion (Example 39).

### Examples 40-42

### BHA/ODA Polymer Dispersed in an Orthodontic Adhesive

Dispersions of thermally responsive polymers independently added to an orthodontic adhesive were prepared by combining BHA/ODA Polymers (2.5 wt.-%; various weight ratios; see Starting Materials Preparations) and APC PLUS orthodontic adhesive, and mixing in a Speed Mixer (DAC-150-FVZ) operating at 3000 rpm for 4×30 seconds to provide the dispersions. Polymers employed with weight ratios indicated were BHA/ODA (90:10), BHA/ODA (70:30), and BHA/ODA (50:50) to afford Example 40, Example 41, and Example 42, respectively.

### Examples 43-45

### Wax Materials Dispersed in an Orthodontic Adhesive

Dispersions of thermally responsive wax materials independently added to an orthodontic adhesive were prepared by combining known waxes (2.5 wt.-%) and APC PLUS orthodontic adhesive, and mixing in a Speed Mixer (DAC-150-FVZ) operating at 3000 rpm for 4×30 seconds to provide the dispersions. Wax materials employed were CERIDUST 3719, CERIDUST 3731, and MIWAX 411 to afford Example 43, Example 44 and Example 45, respectively.

### Evaluation K

### Bond Strength Evaluations of Thermally Responsive Additive-Containing Cured Orthodontic Adhesive Compositions (Examples 38-45)

### Debonding of CLARITY Brackets from Bovine Teeth Surfaces

Samples of Example 38 (2.5 wt.-% PCL in APC PLUS), Example 39 (4.0 wt.-% BHA/HDDA polymer in APC PLUS), Examples 40-42 (2.5 wt.-% BHA/ODA polymer in APC PLUS), and Examples 43-45 (2.5 wt.-% wax materials in APC PLUS), and the corresponding APC PLUS orthodontic adhesive without additives (CONTROL designated as “C”) were evaluated for shear bond strength on bovine teeth using the Shear Bond Strength on Teeth Test Method A described herein (with CLARITY brackets; 3M Unitek). For Examples 38 and 39 (data reported in Table 18), the CLARITY brackets were silane treated as described in the Shear Bond Strength on Teeth Test Method B, except that GF-31 (neat silane) was substituted for the 0.5% SCOTCHPRIME ceramic primer. For Examples 40-45 and 38 (data reported in Tables 19 and 20), the CLARITY brackets were not silane treated.

The results (average of 4-5 replicates) are provided in Tables 18-20 and include bond strength and ARI data at room temperature and elevated temperatures. In all cases, the results showed that bond strengths were significantly reduced by using brackets coated with an orthodontic adhesive containing a thermally responsive additive and heated for 1 minute in a water bath set at either 65° C. or 75° and then debonding quickly to avoid heat loss.

TABLE 18
Debonding of Ceramic Brackets from Bovine Teeth Surface.
Shear Bond Strength and ARI Data

| | | | | Bond | | | |
|---|---|---|---|---|---|---|---|
| | | | | Strength | | | |
| | | | | (MPa) | | ARI Data | |
| Ex. | Run | Temp. | Orthodontic Adhesive | Mean | S.D. | Mean | S.D. |
| C | 1 | 25° C. | APC PLUS | 33.2 | 6.5 | 0.7 | 1.2 |
| 38 | 2 | 75° C. | APC PLUS + PCL | 14.0 | 3.7 | 0.0 | 0.0 |
| | | (1 min.) | (2.5%) | | | | |
| 39 | 3 | 75° C. | APC PLUS + BHA/ | 20.0 | 5.7 | 0.0 | 0.0 |
| | | (1 min.) | HDDA Polymer | | | | |
| | | | (2.5%) | | | | |

TABLE 19
Debonding of Ceramic Brackets from Bovine Teeth Surface.
Shear Bond Strength and ARI Data at Room Temperature (RT) and
75° C. (1 min.)

| | | | | Bond | | | |
|---|---|---|---|---|---|---|---|
| | | | | Strength | | | |
| | | | | (MPa) | | ARI Data | |
| Ex. | Run | Temp. | Orthodontic Adhesive | Mean | S.D. | Mean | S.D. |
| C | 1 | RT | APC PLUS | 11.9 | 3.9 | 3.0 | 0.0 |
| 40 | 2 | RT | APC PLUS + BHA/ODA (90/10) Polymer (2.5%) | 9.7 | 2.0 | 3.0 | 0.0 |
| 41 | 3 | RT | APC PLUS + BHA/ODA (70/30) Polymer (2.5%) | 10.9 | 1.6 | 3.0 | 0.0 |
| 42 | 4 | RT | APC PLUS + BHA/ODA (50/50) Polymer (2.5%) | 10.1 | 1.8 | 3.0 | 0.0 |
| 38 | 5 | RT | APC PLUS + PCL (2.5%) | 7.8 | 2.1 | 3.0 | 0.0 |
| 40 | 6 | 75° C. | APC PLUS + BHA/ODA (90/10) Polymer (2.5%) | 6.1 | 2.8 | 3.0 | 0.0 |
| 41 | 7 | 75° C. | APC PLUS + BHA/ODA (70/30) Polymer (2.5%) | 5.0 | 1.0 | 3.0 | 0.0 |
| 42 | 8 | 75° C. | APC PLUS + BHA/ODA (50/50) Polymer (2.5%) | 7.5 | 1.7 | 3.0 | 0.0 |
| 38 | 9 | 75° C. | APC PLUS + PCL (2.5%) | 5.3 | 2.2 | 3.0 | 0.0 |

TABLE 20
Debonding of Ceramic Brackets from Bovine Teeth Surface.
Shear Bond Strength and ARI Data at Room Temperature (RT) and 65° C. (1 min.)

| | | | | Bond | | | |
|---|---|---|---|---|---|---|---|
| | | | | Strength | | | |
| | | | | (MPa) | | ARI Data | |
| Ex. | Run | Temp. | Orthodontic Adhesive | Mean | S.D. | Mean | S.D. |
| C | 1 | RT | APC PLUS | 10.7 | 1.7 | 3.0 | 0.0 |
| 38 | 2 | RT | APC PLUS + PCL (2.5%) | 10.4 | 3.4 | 3.0 | 0.0 |
| 43 | 3 | RT | APC PLUS + CERIDUST 3731 (2.5%) | 12.5 | 1.7 | 3.0 | 0.0 |
| 44 | 4 | RT | APC PLUS + CERIDUST 3719 (2.5%) | 10.4 | 0.8 | 3.0 | 0.0 |
| 45 | 5 | RT | APC PLUS + MIWAX 411 (2.5%) | 11.5 | 3.9 | 3.0 | 0.0 |
| C | 6 | 65° C. | APC PLUS | 10.8 | 2.4 | 3.0 | 0.0 |
| 38 | 7 | 65° C. | APC PLUS + PCL (2.5%) | 7.5 | 1.8 | 3.0 | 0.0 |
| 43 | 8 | 65° C. | APC PLUS + CERIDUST 3731 (2.5%) | 8.3 | 2.7 | 3.0 | 0.0 |
| 44 | 9 | 65° C. | APC PLUS + CERIDUST 3719 (2.5%) | 7.3 | 2.2 | 3.0 | 0.0 |
| 45 | 10 | 65° C. | APC PLUS + MIWAX 411 (2.5%) | 8.6 | 2.4 | 3.0 | 0.0 |

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

What is claimed is:

1. A method for reducing the bond strength of an orthodontic appliance adhered to a tooth structure with a hardened dental composition, the method comprising heating the hardened dental composition to reduce the bond strength, wherein the hardened dental composition comprises a thermally responsive additive.

2. The method of claim 1 wherein the hardened dental composition is a hardened primer, and the orthodontic appliance is further adhered to a primed tooth structure with a hardened orthodontic adhesive.

3. The method of claim 2 further comprising removing the orthodontic appliance from the tooth structure, wherein the hardened orthodontic adhesive is substantially retained on the removed orthodontic appliance.

4. The method of claim 1 wherein the thermally responsive additive is selected from the group consisting of semicrystalline polymers, amorphous polymers, liquid crystals, waxes, and combinations thereof.

5. The method of claim 4 wherein the thermally responsive additive is a polymer selected from the group consisting of poly((meth)acrylics), poly((meth)acrylamides), poly(alkenes), poly(dienes), poly(styrenes), poly(vinyl alcohol), poly(vinyl ketones), poly(vinyl esters), poly(vinyl ethers), poly(vinyl thioethers), poly(vinyl halides), poly(vinyl nitriles), poly(phenylenes), poly(anhydrides), poly(carbonates), poly(esters), poly(lactones), poly(ether ketones), poly(alkylene oxides), poly(urethanes), poly(siloxanes), poly(sulfides), poly(sulfones), poly(sulfonamides), poly(thioesters), poly(amides), poly(anilines), poly(imides), poly(imines), poly(ureas), poly(phosphazenes), poly(silanes), poly(silazanes), carbohydrates, gelatins, poly(acetals), poly(benzoxazoles), poly(carboranes), poly(oxadiazoles), poly(piperazines), poly(piperidines), poly(pyrazoles), poly(pyridines), poly(pyrrolidines), poly(triazines), and combinations thereof.

6. The method of claim 1 wherein the thermally responsive additive is a behenyl acrylate copolymer.

7. The method of claim 1 wherein the thermally responsive additive is in the form of particles, powders, fibers, disks, plates, flakes, tubes, films, or combinations thereof.

8. The method of claim 1 wherein the thermally responsive additive is distributed uniformly throughout the hardened dental composition.

9. The method of claim 1 wherein the thermally responsive additive is concentrated in a portion of the hardened dental composition.

10. The method of claim 9 wherein the portion is near or on at least one surface of the hardened dental composition proximate the tooth structure.

11. The method of claim 1 wherein at least a portion of the hardened dental composition is heated to at least 42° C.

12. The method of claim 1 wherein the hardened dental composition further comprises a radiation-to-heat converter, and heating comprises irradiating the hardened dental composition.

13. A method for reducing the adherence of a hardened dental composition to a tooth structure, the method comprising heating the hardened dental composition to reduce the adherence, wherein the hardened dental composition comprises a thermally responsive additive.

14. A hardenable dental composition comprising:
an ethylenically unsaturated compound;
an initiator; and
a thermally responsive additive selected from the group consisting of semicrystalline polymers, amorphous polymers, and combinations thereof,
wherein the composition, upon hardening, bonds an orthodontic appliance to a tooth structure with a bond strength of at least 7 MPa at room temperature measured using a shear peel test.

15. A hardenable dental composition comprising:
an ethylenically unsaturated compound;
an initiator; and
a thermally responsive additive selected from the group consisting of semicrystalline polymers, amorphous polymers, and combinations thereof,
wherein the composition is selected from the group consisting of orthodontic adhesives, orthodontic primers, orthodontic sealants, orthodontic band cements, and combinations thereof.

16. A hardenable dental composition comprising:
an ethylenically unsaturated compound;
an initiator;
a thermally responsive additive; and
a radiation-to-heat converter.

17. The hardenable dental composition of claim 16 further comprising an ethylenically unsaturated compound with acid functionality, and wherein the hardenable dental composition is a self-etching orthodontic primer or a self-etching orthodontic adhesive.

18. A packaged article comprising:
an orthodontic appliance having a base for bonding the appliance to a tooth structure;
a hardenable dental composition on the base of the appliance, wherein the hardenable dental composition comprises an ethylenically unsaturated compound, an initiator, a filler, and a thermally responsive additive; and
a container at least partially surrounding the orthodontic appliance having the hardenable dental composition thereon.

19. The packaged article of claim 18 wherein the hardenable dental composition further comprises a radiation-to-heat converter.

20. An article comprising:
an orthodontic appliance having a base for bonding the appliance to a tooth structure; and
a hardened dental composition on the base of the appliance, wherein the hardened dental composition comprises a thermally responsive additive selected from the group consisting of semicrystalline polymers, amorphous polymers, and combinations thereof.

21. The article of claim 20 wherein the article further comprises one or more layers of different hardenable and/or hardened dental compositions.

22. A method for bonding an orthodontic appliance to a tooth structure, the method comprising:
providing an orthodontic appliance having a base for bonding the appliance to a tooth structure;
providing a hardenable dental composition comprising an ethylenically unsaturated compound, an initiator, a filler, and a thermally responsive additive selected from the group consisting of semicrystalline polymers, amorphous polymers, and combinations thereof;
contacting the hardenable dental composition with the tooth structure and the base of the orthodontic appliance; and
hardening the hardenable dental composition.

23. The method of claim 22 wherein contacting the hardenable dental composition with the tooth structure and the base of the orthodontic appliance comprises:
applying the hardenable dental composition to the tooth structure; and
contacting the base of the orthodontic appliance with the hardenable dental composition on the tooth structure.

24. The method of claim 22 wherein contacting the hardenable dental composition with the tooth structure and the base of the orthodontic appliance comprises:
providing an orthodontic appliance having the hardenable dental composition on the base thereof; and
applying the base of the appliance having the hardenable dental composition thereon to the tooth structure.

25. The method of claim 24 wherein the orthodontic appliance is provided as a precoated appliance having the hardenable dental composition on the base of the appliance.

26. The method of claim 25 wherein the precoated appliance further comprises one or more layers of different hardenable and/or hardened dental compositions.

27. The method of claim 24 wherein providing the orthodontic appliance having the hardenable dental composition on the base thereof further comprises applying the hardenable dental composition to the base of an orthodontic appliance.

28. The method of claim 22 wherein the hardenable orthodontic adhesive further comprises a radiation-to-heat converter.

29. A method for bonding an orthodontic appliance to a tooth structure, the method comprising:
providing an orthodontic appliance having a base for bonding the appliance to a tooth structure, wherein the appliance, on the base thereof, has a hardened dental composition comprising a thermally responsive additive selected from the group consisting of semicrystalline polymers, amorphous polymers, and combinations thereof,
providing a hardenable orthodontic adhesive;
contacting the hardenable orthodontic adhesive with the tooth structure and the base of the orthodontic appliance having the hardened dental composition thereon; and
hardening the orthodontic adhesive.

30. The method of claim 29 wherein contacting the hardenable orthodontic adhesive with the tooth structure and the base of the orthodontic appliance having the hardened dental composition thereon comprises:
applying the hardenable orthodontic adhesive to the tooth structure; and
contacting the base of the orthodontic appliance having the hardened dental composition thereon with the hardenable orthodontic adhesive on the tooth structure.

31. The method of claim 29 wherein contacting the hardenable orthodontic adhesive with the tooth structure and the base of the orthodontic appliance having the hardened dental composition thereon comprises:
providing an orthodontic appliance having the hardenable orthodontic adhesive on the hardened dental composition on the base thereof; and
applying the base of the appliance having the hardenable orthodontic adhesive on the hardened dental composition thereon to the tooth structure.

32. The method of claim 31 wherein the orthodontic appliance is provided as a precoated appliance having the hardenable orthodontic adhesive on the hardened dental composition on the base of the orthodontic appliance.

33. The method of claim 32 wherein the precoated appliance further comprises one or more layers of different hardenable and/or hardened dental compositions.

34. The method of claim 31 wherein providing the orthodontic appliance having the hardenable orthodontic adhesive on the hardened dental composition on the base thereof further comprises applying the hardenable orthodontic adhesive to the hardened dental composition on the base of the orthodontic appliance.

35. A method for bonding an orthodontic appliance to a tooth structure, the method comprising:
providing a tooth structure having a hardened dental composition comprising a thermally responsive additive thereon;
providing an orthodontic appliance having a base for bonding the appliance to a tooth structure;
providing a hardenable orthodontic adhesive;
contacting the hardenable orthodontic adhesive with the base of the orthodontic appliance and the tooth structure having the hardened dental composition thereon; and
hardening the orthodontic adhesive.

36. The method of claim 35 wherein providing a tooth structure having a hardened dental composition thereon further comprises:
applying a dental composition comprising a thermally responsive additive to the tooth structure; and
hardening the dental composition.
